(19) United States
(12) Patent Application Publication
(10) Pub. No.: US 2017/0114353 A1
(43) Pub. Date: Apr. 27, 2017

(54) REGULATION OF NITRATE UPTAKE AND NITROGEN USE BY BTB GENES

(71) Applicants: PONTIFICIA UNIVERSIDAD CATOLICA DE CHILE, Santiago (CL); FUNDACION COPEC-UNIVERSIDAD CATÓLICA CENTRO INNOVACION UC ANACLETO ANGELINI PISO 10, Santiago (CL)

(72) Inventors: Rodrigo Antonio Gutiérrez Ilabaca, Santiago (CL); Erika Viviana Araus Caramori, La Florida (CL); Elena Alejandra Vidal Olate, Stgo (CL)

(21) Appl. No.: 14/921,709

(22) Filed: Oct. 23, 2015

(51) Int. Cl. C12N 15/82; C07K 14/415; C12N 15/113

(52) U.S. Cl. CPC C12N 15/8218; C12N 15/8243; C12N 15/1138; C07K 14/415; A23L 1/212; C12N 2310/11; A23V 2002/00

(57) ABSTRACT

This disclosure concerns the regulation of nitrogen use efficiency in plants. Embodiments concern regulatory factors that contribute to the growth phenotype of plants in limited nitrogen conditions.

### FIELD OF THE DISCLOSURE

The present disclosure relates to plant biochemistry. Embodiments relate to genetic factors regulating nitrogen use efficiency in plants.

### BACKGROUND

Nitrogen is an essential macronutrient and a major limiting factor for plant growth, development, and productivity. Marschner (1995) Mineral Nutrition of Higher Plants, Academic Press, Harcourt, San Diego, Calif., p. 889; Epstein (2005) Mineral Nutrition of Plants: Principles and Perspectives, 2^{nd} Ed., Sinauer Associates, Inc., Sunderland, Mass.; Galloway and Cowling (2002) AMBIO 31:64. Traditional agriculture is based on nitrogen fertilizers to support world nutritional needs. Thus, the use of nitrogen-based fertilizers has increased more than 8-fold in the last 50 years to cope with increasing demands of agriculture and food production. Dawson & Hilton (2011) Food Policy 36(S1): 14.

However, intensive use of nitrogen-fertilizers is having a major detrimental impact on the ecosystem and to human health, including eutrophication of waters, and increase of gaseous emissions of toxic nitrogen oxides and ammonia to the atmosphere. Ju et al. (2009) Proc. Natl. Acad. Sci. USA 106(9):3041-6 (correction at Proc. Natl. Acad. Sci. USA 106(19):8077); Lassaletta et al. (2014) Biogeochemistry 118:225-41; and Robertson & Vitousek (2009) Ann. Rev. Environ. Resources 34(1):97-125. Moreover, the use of nitrogen-fertilizers is a major cost for farmers, which in turn affects the commercial price of vegetables and fruits.

Nitrogen use efficiency (NUE) is a complex genetic trait and index that encompasses multiple metabolic, physiological, and developmental processes in plants exposed to a changing environment. Processes that govern NUE are broadly divided into two main categories: nitrogen uptake; and nitrogen utilization efficiency, including assimilation, internal nitrogen transport, and nitrogen remobilization. Hirel et al. (2007) J. Exp. Bot. 58(9):2369-87; Gallais & Hirel (2004) 1 Exp. Bot. 55(396):295-306; Masclaux-Daubresse et al. (2010) Ann. Bot. 105(7):1141-57; Bi et al. (2009) Plant Cell Environ. 32(12):1749-60; Xu et al. (2012) Ann. Rev. Plant Biol. 63:153-82. NUE has been defined in various ways (Good et al. (2004) Trends Plant Sci. 9(12):597-605), but yield (measured by grain, fruit or forage depending on the crop) per unit of nitrogen available in the soil integrates all key parameters for evaluating fitness of crop cultivars and it is a common measure of NUE (Moll et al. (1982) Agronomy J. 74(3):562; Kant et al. (2011) J. Exp. Bot. 62(4):1499-1509; Beatty et al. (2010) Ann. Bot. 105(7):1171-82; Gupta et al. (2012) Scientific World Journal 2012:625731). Integrated nitrogen management strategies and overall better agricultural practices improved NUE over the last years. Jing et al. (2009) J. Agr. Sci. 147(3):303.

Many efforts are currently devoted towards defining target genes for generating crops with enhanced NUE. Crawford & Forde (2002) Arabidopsis Book 46(3):1. Due to its essential role in N assimilation, glutamine synthetase (GS) has been a prime target gene to improve NUE. Numerous studies reported overexpression of GLUTAMINE SYNTHETASE 1 (GS1), the cytosolic isoform of GS, to improve NUE in different species such as tobacco, maize, rice and Arabidopsis. Eckes et al. (1989) Mol. Gen. Genet. 217:263-8; Migge et al. (2000) Planta 210(2):252-60; Man et al. (2011) J. Exp. Bot. 62(13):4423-31. Overexpression of GS1 showed positive effects on plant productivity in a few cases. Habash & Massiah (2001) Ann. Appl. Biol. 138(1):83-9; Martin et al. (2006) Plant Cell 18(11):3252-74; Obara et al. (2004) Theor. Appl. Genet. 110(1):1-11.

During N assimilation, GS works together with glutamine oxoglutarate aminotransferase (GOGAT). Suppression of GOGAT isozymes (Fd-GOGAT and NADH-GOGAT) causes a decrease in tiller number, shoot dry weight and yield in rice. Lu et al. (2011) Sci. China Life Sci. 54(7):651-63.

Besides genes directly involved in N metabolism, overexpression of genes including the sugar transport protein STP13 of Arabidopsis (Schofield et al. (2009) Plant Cell Environ. 32(3):271-85) the early nodulin gene (OsENOD93-1) (Bi et al. (2009), supra) and the peptide transporter/nitrate OsPTR9 (Fang et al. (2013) Plant Biotechnol. J. 11(4):446-58) of rice has been shown to positively affect production traits of the plants, as biomass, grain yield or nitrogen content.

In addition, over-expression of the transcription factor DOF1 in Arabidopsis and rice resulted in plants with increased amino acid content, increased carbon skeleton production and a reduction in glucose levels, suggesting a possible role for DOF1 in NUE. Yanagisawa et al. (2004) Proc. Natl. Acad. Sci. USA 101 (20):7833-8.

While all these genes impact processes that are related to NUE, it is unclear whether alteration in expression of these genes leads to measurable changes in plant NUE.

### BRIEF SUMMARY OF THE DISCLOSURE

To sustain increasing demands for plant food due to a growing population, new agricultural practices are required to reduce the dependency on nitrogen fertilizers. In order to understand regulatory mechanisms underlying the utilization efficiency of environmental nitrogen, a systems biology approach to identify genes involved in the regulation of NUE was utilized. Members of the Bric-a-Brac/Tramtrack/Broad (BTB) gene family (e.g., bt1 and bt2) were identified as negative regulators of gene expression, nitrate uptake, and NUE.

Disclosed herein are isolated, synthetic, and/or recombinant nucleic acid molecules comprising a polynucleotide operably linked to a heterologous promoter, wherein the polynucleotide encodes a BTB polypeptide. In some embodiments, the polypeptide may be a BTB1 or BTB2 polypeptide. In particular embodiments, the polypeptide may be, for example, at least 80% identical to an amino acid sequence selected from the group consisting of SEQ ID NO:2, SEQ ID NO:4, SEQ ID NO:12, SEQ ID NO:14, and SEQ ID NO:16. In particular embodiments, the polypeptide may be homolog or ortholog of the foregoing polypeptides, for example, having an amino acid sequence that is at least 40% identical to an amino acid sequence selected from the group consisting of SEQ ID NO:2, SEQ ID NO:4, SEQ ID NO:12, SEQ ID NO:14, and SEQ ID NO:16. In specific examples, the polypeptide is at least 85% identical to SEQ ID NO:2 or SEQ ID NO:4. Also disclosed herein are non-natural nucleic acid molecules comprising a polynucleotide operably linked to a heterologous promoter, wherein the polynucleotide encodes a mutant BTB polypeptide, or an antisense RNA molecule that inhibits the expression of a BTB gene.

Some embodiments include isolated synthetic, and/or recombinant nucleic acid molecules comprising a polynucleotide operably linked to a heterologous promoter, wherein the polynucleotide encodes a BTB1 or BTB2 polypeptide; for example, a polypeptide that is at least 40% identical (e.g., at least 80%, 85%, 90%, 95%, or 98% identical) to an amino acid sequence selected from the group consisting of SEQ ID NO:2, SEQ ID NO:4, SEQ ID NO:12, SEQ ID NO:14, and SEQ ID NO:16. In particular embodiments, the polynucleotide operably linked to a heterologous promoter is selected from the group of BTB1 and BTB2 genes and their orthologs and homologs, wherein the group consists of a polynucleotide that is at least 40% identical (e.g., at least 80%, 85%, 90%, 95%, or 98% identical) to any of SEQ ID NOs:1, 3, 11, 13, and 15, or the complement or reverse complement thereof; and a polynucleotide that hybridizes under stringent (e.g., highly stringent) conditions to a nucleic acid consisting of any of SEQ ID NOs:1, 3, 11, 13, and 15, or the complement or reverse complement thereof. Specific examples include an isolated, synthetic, and/or recombinant nucleic acid molecule comprising a polynucleotide operably linked to a heterologous promoter, wherein the polynucleotide is selected from the group consisting of a polynucleotide that is at least 40% identical (e.g., at least 80%, 85%, 90%, 95%, or 98% identical) to SEQ ID NO:1 or the complement or reverse complement thereof; a polynucleotide that hybridizes under stringent (e.g., highly stringent) conditions to a nucleic acid consisting of SEQ ID NO:1 or the complement or reverse complement thereof; a polynucleotide that is at least 40% identical (e.g., at least 80%, 85%, 90%, 95%, or 98% identical) to SEQ ID NO:3 or the complement or reverse complement thereof; and a polynucleotide that hybridizes under stringent (e.g., highly stringent) conditions to a nucleic acid consisting of SEQ ID NO:3 or the complement or reverse complement thereof.

Also disclosed herein are methods for increasing NUE in a plant. In some embodiments, the method may comprise introducing at least one heterologous polynucleotide into a plant cell, wherein the heterologous polynucleotide encodes a mutant BTB polypeptide, or an antisense RNA molecule that inhibits the expression of a BTB gene. In particular embodiments, the BTB polypeptide or BTB gene is btb1 (bt1) or btb2 (bt2). In particular embodiments, the method further comprises introducing into the plant cell a second heterologous polynucleotide encoding a mutant BTB polypeptide, or an antisense RNA molecule that inhibits the expression of a BTB gene, such that the plant cell comprises both btb1 (bt1) and btb2 (bt2), antisense RNA molecules that inhibit the expression of both BT1 and BT2, or combinations of the foregoing that target both BTB1 and BTB2. In particular embodiments, the plant cell is cultured to produce a transgenic plant comprising the heterologous polynucleotide.

In some embodiments, a method for increasing NUE in a plant may comprise transforming a plant cell with a nucleic acid molecule comprising the heterologous polynucleotide. In some embodiments, the heterologous polynucleotide may be substantially identical to all or part of the reverse complement of a polynucleotide encoding SEQ ID NO:2 or SEQ ID NO:4 (e.g., the reverse complements of SEQ ID NO:1 and SEQ ID NO:3) and/or a homologous or orthologous polypeptide thereof. For example, the heterologous polynucleotide may be substantially identical to at least 18 contiguous nucleotides of the reverse complement of SEQ ID NO:1 or SEQ ID NO:3. In some embodiments, the heterologous polynucleotide encodes a ribonucleic acid (RNA) molecule that hybridizes under stringent (e.g., highly stringent) conditions to the transcription product of a BTB gene (e.g., bt1 and bt2). In particular embodiments, the transformed plant cell is cultured to produce a transgenic plant comprising the heterologous polynucleotide.

In some embodiments, a method for increasing NUE in a plant may comprise transforming a plant cell with a nucleic acid molecule comprising a heterologous polynucleotide that encodes a mutant BTB polypeptide. In some embodiments, the heterologous polynucleotide may encode a truncated protein, it may comprise non-functional regulatory sequences that alter or disrupt expression of the BTB polypeptide, or it may comprise one or more mutations (e.g., insertion, deletion, and point mutations) that render the BTB polypeptide; for example, frame-shift mutations, exon deletions, and/or alteration of one or more amino acids that are conserved among members of the BTB protein family. In some embodiments, the heterologous polynucleotide further comprises sequences flanking the coding region, such that (following transformation of a target plant cell with the nucleic acid molecule) replacement of a native, genomic BTB gene by the heterologous polynucleotide is directed by the process of homologous recombination. In particular examples, a mutant BTB polypeptide may be, for example, at least 40% identical (e.g., at least 80%, 85%, 90%, 95%, or 98% identical) to SEQ ID NO:8 or SEQ ID NO:10. In particular embodiments, a method for increasing NUE in a plant may comprise generating a plant mutant or genetically modified plant that lacks or limits the expression of the BTB protein family or polypeptides that are at least 40% identical (e.g., at least 80%, 85%, 90%, 95%, or 98% identical) to SEQ ID NO:2 or SEQ ID NO:4. In particular embodiments, this mutant trait incorporated in a plant breeding program to improve NUE.

In some embodiments, a method for increasing NUE in a plant may comprise reducing the expression of a BTB gene in a plant. In particular embodiments, a CRISPR-based genetic engineering system is utilized to mutate a BTB gene in a plant genome.

Also disclosed herein are methods for increasing NUE in a plant comprising introducing into a plant cell at least one at least one means for silencing bt1 expression in a plant, and/or at least one means for silencing bt2 expression in a plant, for example, to produce a transgenic plant. Examples of means for silencing bt1 expression in a plant include a polynucleotide consisting of SEQ ID NO:5 and a polynucleotide consisting of SEQ ID NO:7. Functional equivalents of SEQ ID NO:5 include, for example and without limitation, antisense iRNA molecules targeting a bt1 gene. Examples of means for silencing bt2 expression in a plant include a polynucleotide consisting of SEQ ID NO:6 and a polynucleotide consisting of SEQ ID NO:9. Functional equivalents of SEQ ID NO:6 include, for example and without limitation, antisense iRNA molecules targeting a bt2 gene.

Also disclosed herein are transgenic plant materials (e.g., plant cells, plant parts, plant tissues, plant tissue cultures, plant seeds, and whole plants) comprising, or stably transfoinied with, any of the foregoing polypeptides, polynucleotides, and/or nucleic acid constructs. Such a transgenic plant material in particular embodiments exhibits increased growth under limited nitrogen growing conditions, as compared to a plant of the same species that does not comprise the polypeptide, polynucleotide, and/or nucleic acid construct.

The foregoing and other features will become more apparent from the following detailed description of several embodiments, which proceeds with reference to the accompanying figures.

### BRIEF DESCRIPTION OF THE FIGURES

FIG. 1 includes a representation of a NUE network of 350 genes predicted by DLS analysis. The genes in this network are targets for improving NUE in plants. BT2 was selected as a representative gene in the NUE network, as statistical methods applied over each node in the network highlights BT2 as the central hub of the network.

FIGS. 2A-2D include graphical representations of the relationship between nitrate concentration and NUE. FIG. 2A shows the relationship between NUE (g seed/g N) and nitrate concentration (mM) in wild-type plants. FIG. 2B shows the relationship between the productivity (g seed) and nitrate concentration (mM) in wild-type plants. FIG. 2C shows the relationship between NUE and nitrate concentration in wild-type (Col-0) and plants overexpressing the BT2 gene (BT2OE). FIG. 2D shows the relationship between NUE and nitrate concentration in wild-type (Col-0×Ler) and bt1/bt2 mutant plants (bt1bt2). Shown are the mean and standard error for three independent biological replicates with 12 plants each. Asterisks represent means that statistically differ (p<0.0⁵).

FIGS. 3A-3C include data showing that altered expression of BT1 and BT2 affects plant biomass under low nitrate availability during the entire life cycle of the plant. FIG. 3A shows the relationship between biomass (plant dry weight) and nitrate concentration in wild-type (Col-0) and BT2 overexpressing (BT2OE) plants. FIG. 3B shows the relationship between biomass and nitrate concentration in wild-type (Col-0×Ler) and bt1/bt2 mutant plants (bt1bt2). Shown are the mean and standard error for three independent biological replicates with at least 12 plants each. Asterisks represent means that statistically differ (p<0.05). FIG. 3C shows images of two-week old wild-type, BT2 overexpressing, and bt1/bt2 mutant seedlings grown under 0.5 mM KNO₃.

FIGS. 4A-4C include data showing that BT1 and BT2 reduce expression of NRT2.1 and NRT2.4 genes (and thereby nitrate uptake) under conditions of low nitrate availability. FIG. 4A shows NRT2.1 transcript levels as a function of nitrate concentration in wild-type (Col-0; Col-0×Ler), BT2 overexpressing (BT2OE), and bt1/bt2 mutant (bt1bt2) seedlings. FIG. 4B shows NRT2.4 transcript levels as a function of nitrate concentration in wild-type, BT2 overexpressing, and bt1/bt2 mutant seedlings. FIG. 4C shows the relationship between the relative nitrogen uptake (total ¹⁵N content) and nitrate concentration in BT2 overexpressing and bt1/bt2 mutant seedlings. Shown are the mean and standard error for three independent biological replicates with at least 12 plants each. Asterisk represents means that statistically differ (p<0.05).

FIGS. 5A and 5B include data showing that the single mutation of either of BT1 and BT2 affects NUE only within the context of a further mutation. FIG. 5A shows the relationship between NUE and nitrate concentration in wild-type (Col-0) and bt2 mutant (bt2-1) plants. FIG. 5B shows the relationship between NUE and nitrate concentration in wild-type (Ler) and bt1 mutant (bt1-4) plants. Shown are the mean and standard error for two independent biological replicates with at least 8 plants.

FIGS. 6A-6C include data showing that altered expression of BT1 and BT2 genes affects early stages of vegetative development. FIGS. 6A-6C show the relationship between the day of appearance of true leaves (FIG. 6A), the number of rosette leaves with abaxial trichomes (FIG. 6B), and the day of bolting (FIG. 6C) and nitrate concentration in wild-type (Col-0; Col-0×Ler), BT2 overexpressing (BT2OE), and bt1/bt2 mutant (bt1bt2) seedlings. Shown are the mean and standard error for three independent biological replicates with at least 15 plants each.

### SEQUENCE LISTING

The nucleic acid sequences listed in the accompanying sequence listing are shown using standard letter abbreviations for nucleotide bases, as defined in 37 C.F.R. §1.822. Only one strand of each nucleic acid sequence is shown, but the complementary strand is understood to be included by any reference to the displayed strand. In the accompanying sequence listing:

SEQ ID NO:1 shows an exemplary polynucleotide encoding a BTB1 polypeptide:

CCATCATCAAATTAAAACCCTCCAGCCTCCAACAACAAAATTTACCAAC
ACTCTCTCTTTCTCTTTCTCTCTGAAAACTTAAAACGATGGCTATAACC
GCCACTCAAAACGACGGCGTTTCACTAAACGCCAATAAGATTTCGTATG
ATCTTGTGGAAACTGATGTTGAGATCATCACCTCCGGTCGTCGTAGTAT
TCCGGCACACTCCGGCATCCTCGTAAGTTTCTTTTCTTCTTACTTTCTT
TGTATGATCGAATATTTTTTTATTTTGGTGCTTATCTTTTTTGTTTGTT
TAAAATTGTTAAATGCAGGCTTCGGTCTCACCGGTACTGACGAACATCA
TCGAGAAGCCGAGGAAGATTCACGGCGGATCATCGAAGAAAGTTATTAA
GATTCTCGGTGTTCCATGCGACGCCGTTTCAGTCTTCGTCAGATTCCTC
TATTCTCCGAGGTTCGTCTTTCTCTCTTTTTCATATCTTCTTGATCGTC
AGATTTTTTTTTTTAATCCGTAGATTTATTACTTCAAAAATACAAATCA
TACTAAAAAGAATGCTTGATTAAATACATCATCTTAAAGATCTTTACCA
AACTCGACTTTTCCAACGTTTTAATCATACATTAAAATCACTACAACCA
GATTGCGATTGATTTAGACTTGTTTCATATTCAAAAACGTGTGAAACAC
AACACAAGGAACACGAAACTAGTACCAACATCAAATTTGAATAAATCAA
CGGCAAATTTTACCCTTAGTATGCAAAATGACTAGAATACCCCTATATG
TATCTTTTGTTTAGTGTCACGGAGAATGAGATGGAAAAATACGGAATCC
ATCTACTGGCTTTATCACACGTGTATATGGTGACTCAGTTAAAGCAACG
GTGCACTAAAGGCGTCGGTGAGAGAGTAACCGCCGAAAACGTTGTCGAT
ATTCTCCAGCTGGCTCGTCTCTGCGACGCACCTGACCTCTGTCTCAAGT
GTATGCGATTCATTCACTACAAGTTCAAGACCGTTGAACAGACAGAAGG
ATGGAAGTTTCTTCAAGAACACGATCCTTTTCTTGAACTTGACATTCTC
CAATTCATCGACGATGCAGAATCGGTAATTAACTCAAACCACACTTTTT
TGTTTTCTTTTTTTTTGGTTCTTCAAGAAAACTCTGTTTTATTGAATTA
GGTTTCTTTTGGGTCAATTTCTTGCAGAGGAAGAAAAGAAGAAGGAGAC
ACAGACGAGAACAGAATCTGTATTTGCAGCTGAGTGAAGCCATGGAATG
TATAGAACACATATGCACCGAAGGTTGCACACTGGTCGGACCATCGTCT
AACTTAGACAACAAGTCAACATGTCAAGCAAAACCCGGTCCATGCAGTG
CGTTTTCGACTTGTTACGGACTTCAACTTCTTATACGTCACTTTGCAGT
ATGCAAGAAAAGAGTCGATGGCAAAGGTTGTGTCCGATGCAAGAGAATG
ATTCAACTCCTTAGACTCCATTCTTCGATTTGTGACCAATCTGAATCTT
GCCGTGTCCCTCTTTGCAGGTAAAAATCCGACATTTTAGAATAATCAAA
ACCACATTTTAAATGTTTCTAATGATAAAAGTTTTTCACTTTTCGATAG
TAGATCTCATTTGATTTTTTTTTAACTTTCGGCGCCAATAATAATTATC
TTTTTGTGGTGGTAGCCTACTTATTAGATGATGAGTCGTGATCGGACGG
CTGGAAATATTTTGTTTGGTAGAATTATTAAAAGATATTTTAAGAAATG
AGAATCTGATCTCGTTGTCAACTTTTTATTTGTTTTGGTGATTGGAGTA
GGCAATATAAGAATAGAGGTGAAAAGGACAAGAAAATGGTTGAGGACAC
GAAGTGGAAGGTTCTGGTGAGAAGAGTAGCGTCTGCTAAAGCCATGTCT
TCGTTGTCTCAATCAAAGAAGAAAAAAAGTGAAGTGTTATTTAAAGAAG
AAGCAGAAGATTTGATCAGAATCCGGAACAAGTTAATGTGAATATACAA
TATATGTGTTTTTATTAGATTATATAGTGAGCGATTGTTAGAAGCCATT
AATTACTTGAGTCGGAAGGTTTATTTGTTTGTTATTTGTCTCTCGGACT
GGTTGATTGGGTTAAACTTGTCAATGAGTAACCCTTTGAATCTGTCGTT
TTGTTTGGTCGGTCGAATTATATAGAAATGTATTATTGGTTATAAAATG
GTATTAGGGTTTTTGGAG

SEQ ID NO:2 shows an exemplary BTB1 polypeptide:

MAITATQNDGVSLNANKISYDLVETDVEIITSGRRSIPAHSGILASVSP
VLTNIIEKPRKIHGGSSKKVIKILGVPCDAVSVFVRFLYSPSVTENEME
KYGIHLLALSHVYMVTQLKQRCTKGVGERVTAENVVDILQLARLCDAPD
LCLKCMRFIHYKFKTVEQTEGWKFLQEHDPFLELDILQFIDDAESRKKR
RRRHRREQNLYLQLSEAMECIEHICTEGCTLVGPSSNLDNKSTCQAKPG
PCSAFSTCYGLQLLIRHFAVCKKRVDGKGCVRCKRMIQLLRLHSSICDQ
SESCRVPLCRQYKNRGEKDKKMVEDTKWKVLVRRVASAKAMSSLSQSKK
KKSEVLFKEEAEDLIRIRNKLM

SEQ ID NO:3 shows an exemplary polynucleotide encoding a BTB2 polypeptide:

CAAACTACCAAACCTACAGAAATCTCTCTTCTTCTTCTTCTCTACAAAA
TTTCAATGGAAGCTGTTCTTGTCGCAATGTCCGTCCCCGCCACAACGGA
AGACGACGGATTTTCGTTAATCACCGATAAACTTTCATATAATCTAACG
CCGACGTCGGACGTTGAGATCGTTACCTCCGATAACCGTCGGATTCCGG
CACACTCCGGCGTTCTGGTAAGTTGTTATTTATTTTGGTTTACGGTTTA
CATTTTTCTGGTTTGGTTTTTAGCTAAAATTTGTGGATAATGCAGGCTT
CAGCTTCACCGGTACTGATGAACATCATGAAGAAACCGATGAGACGTTA
CAGAGGCTGTGGATCTAAAAGAGTCATCAAGATTCTTGGCGTTCCATGC
GACGCCGTTTCGGTTTTTATCAAATTCCTCTACTCTTCTAGGTTGGTTT
GGTTCGGTTATTCTTCTTGAATTTTCCAAATGTAGAAACTTCAAACCGA
TGGTTATTTCCGGTACTACACAATTTTCCCGGTTTTTCTTTTTCATACA
CGGATTTGAAGAAACTCTGTTTTTTTTTGGGCTATTTTCTTGGGGAAAC
AATAGCTGTCAGGAAATGACTAAACTACCCTTTTGCGTCTCTGTTTTAG
TTTGACGGAGGATGAGATGGAGAGATATGGAATCCATCTTTTAGCGTTG
TCTCACGTCTACATGGTCACCCAGCTGAAGCAACGGTGCTCAAAAGGCG
TTGTACAACGTTTAACTACAGAGAACGTAGTCGACGTCCTCCAGCTAGC
TCGGCTCTGCGACGCACCCGATGTTTGCCTTCGATCTATGCGTCTGATC
CATTCGCAGTTTAAGACCGTTGAGCAGACTGAAGGATGGAAGTTTATTC
AAGAACATGATCCTTTCCTCGAGCTCGACATTCTCCAGTTCATCGATGA
CGCCGAATCGGTAAACTTACTTATATGTTTTGAAAACACAGCATCTCTG
TTTTTCTTAGTACCTTCGATAACACATTTTGTTCAGCTAGGTTTAGTTT
TTGTTCCGCGCTAGTAAAAAGATAATTTTTTTTTTTTTAAATTCAGTTT
ATTTAGTTTGGCATGTTGCACAAAGTTTAGAGTATTAGTTAACTCATGT
TTGATTAGATTTGGTTTATATACCTAATTAACCCCAAAACATTTGGTCA
TTTTTCTGATTATGTGTTGATTTATAATTTAACGCAGAGGAAGAAAAGA
AGGCGACGACATAGAAAGGAGCAGGATCTTTACATGCAACTAAGCGAGG
CAATGGAGTGCATAGAGCACATATGCACACAAGGTTGTACATTGGTCGG
TCCATCAAACGTAGTAGATAACAACAAAAAGTCAATGACTGCGGAAAAG
TCAGAGCCTTGTAAGGCGTTTTCCACGTGTTATGGTCTTCAGCTTTTGA
TACGTCACTTTGCCGTATGCAAGAGGAGGAATAACGATAAGGGTTGTCT
TCGTTGCAAGCGGATGCTTCAACTCTTTAGACTCCATTCTTTGATTTGT
GATCAACCCGATTCTTGTCGCGTCCCTCTCTGCAGGTATGTCCCAACTT
GTTTTGTTAGGAATTTCACATTTGAAATAAATCAAAAAGTATGAGCGAA
TCCATTTACGCTTAATTAGTAAGTTTGAAATTGATGATTTTTAGTATGT
TTATGAATAAAAACCAATGTTTTGTTGAAAGTGAAAAAATTTCATTTTT
GGTAAGAGACAAGGTTTGTTTATCTTTTTGTGTAGACCCACCTTTAGAA
AAGTTGCAAATTGCTATGTGAATTGATGATGGGTCGTAATCCGACGGCT
GCAAATTGTTGCCATTTCTTGAAATTTGTAGATATTTTAATAGAATCTC
ATGTGTTCTTTTCAACTCTTTTTTTTGGGACTAGGCAATTTAGGAAACG
GGGAGAACAAGACAAGAAAATGOGTGAAGACACCAAGTGGAAGCTTCTG
GTGACAAGGGTTGTGTCTGCAAAAGCTATGACGTCGTTGTGTCAGTCAA
AGAAGAACAAATGTGAACAAGCACAAGGGGTTTAATCAAAACCAGGAAG
GAGTTAGTTAGGTTTGAAGAAGATTAAGTAAGAAGCTTTCTTATAATTT
GATATCCATTCTTTTTGTAGTATAATAGTATTTGTTAGTTCGATTCTTT
ATTAGAGGGGTTTAGTTTGTATAGGAGTAAACCCTTTTAAATTGGCTGT
ATAAGTAGGTCATATTCAATTGTTTTGTTCTATTTATCTGAAAAATGTC
AGCATGTAATACAAAACAAATAAATGTTCAAATTAAAGGGAGGATTACA
AGAGGTGTTTTTAATGTTATTGACTTTGGCC

SEQ ID NO:4 shows an exemplary BTB2 polypeptide:

MEAVLVAMSVPATTEDDGFSLITDKLSYNLTPTSDVEIVTSDNRRIPAH
SGVLASASPVLMNIMKKPMRRYRGCGSKRVIKILGVPCDAVSVFIKFLY
SSSLTEDEMERYGIHLLALSHVYMVTQLKQRCSKGVVQRLTTENVVDVL
QLARLCDAPDVCLRSMRLIHSQFKTVEQTEGWKFIQEHDPFLELDILQF
IDDAESRKKRRRRHRKEQDLYMQLSEAMECIEHICTQGCTLVGPSNVVD
NNKKSMTAEKSEPCKAFSTCYGLQLLIRHFAVCKRRNNDKGCLRCKRML
QLFRLHSLICDQPDSCRVPLCRQFRKRGEQDKKMGEDTKWKLLVTRVVS
AKAMTSLCQSKKNKCEQAQGV

SEQ ID NO:5 shows the reverse complement of the exemplary BT1 polynucleotide of SEQ ID NO: 1.

CCATCATCAAATTAAAACCCTCCAGCCTCCAACAACAAAATTTACCAAC
ACTCTCTCTTTCTCTTTCTCTCTGAAAACTTAAAACGATGGCTATAACC
GCCACTCAAAACGACGGCGTTTCACTAAACGCCAATAAGATTTCGTATG
ATCTTGTGGAAACTGATGTTGAGATCATCACCTCCGGTCGTCGTAGTAT
TCCGGCACACTCCGGCATCCTCGTAAGTTTCTTTTCTTCTTACTTTCTT
TGTATGATCGAATATTTTTTTATTTTGGTGCTTATCTTTTTTGTTTGTT
TAAAATTGTTAAATGCAGGCTTCGGTCTCACCGGTACTGACGAACATCA
TCGAGAAGCCGAGGAAGATTCACGGCGGATCATCGAAGAAAGTTATTAA
GATTCTCGGTGTTCCATGCGACGCCGTTTCAGTCTTCGTCAGATTCCTC
TATTCTCCGAGGTTCGTCTTTCTCTCTTTTTCATATCTTCTTGATCGTC
AGATTTTTTTTTTTAATCCGTAGATTTATTACTTCAAAAATACAAATCA
TACTAAAAAGAATGCTTGATTAAATACATCATCTTAAAGATCTTTACCA
AACTCGACTTTTCCAACGTTTTAATCATACATTAAAATCACTACAACCA
GATTGCGATTGATTTAGACTTGTTTCATATTCAAAAACGTGTGAAACAC
AACACAAGGAACACGAAACTAGTACCAACATCAAATTTGAATAAATCAA
CGGCAAATTTTACCCTTAGTATGCAAAATGACTAGAATACCCCTATATG
TATCTTTTGTTTAGTGTCACGGAGAATGAGATGGAAAAATACGGAATCC
ATCTACTGGCTTTATCACACGTGTATATGGTGACTCAGTTAAAGCAACG
GTGCACTAAAGGCGTCGGTGAGAGAGTAACCGCCGAAAACGTTGTCGAT
ATTCTCCAGCTGGCTCGTCTCTGCGACGCACCTGACCTCTGTCTCAAGT
GTATGCGATTCATTCACTACAAGTTCAAGACCGTTGAACAGACAGAAGG
ATGGAAGTTTCTTCAAGAACACGATCCTTTTCTTGAACTTGACATTCTC
CAATTCATCGACGATGCAGAATCGGTAATTAACTCAAACCACACTTTTT
TGTTTTCTTTTTTTTTGGTTCTTCAAGAAAACTCTGTTTTATTGAATTA
GGTTTCTTTTGGGTCAATTTCTTGCAGAGGAAGAAAAGAAGAAGGAGAC
ACAGACGAGAACAGAATCTGTATTTGCAGCTGAGTGAAGCCATGGAATG
TATAGAACACATATGCACCGAAGGTTGCACACTGGTCGGACCATCGTCT
AACTTAGACAACAAGTCAACATGTCAAGCAAAACCCGGTCCATGCAGTG
CGTTTTCGACTTGTTACGGACTTCAACTTCTTATACGTCACTTTGCAGT
ATGCAAGAAAAGAGTCGATGGCAAAGGTTGTGTCCGATGCAAGAGAATG
ATTCAACTCCTTAGACTCCATTCTTCGATTTGTGACCAATCTGAATCTT
GCCGTGTCCCTCTTTGCAGGTAAAAATCCGACATTTTAGAATAATCAAA
ACCACATTTTAAATGTTTCTAATGATAAAAGTTTTTCACTTTTCGATAG
TAGATCTCATTTGATTTTTTTTTAACTTTCGGCGCCAATAATAATTATC
TTTTTGTGGTGGTAGCCTACTTATTAGATGATGAGTCGTGATCGGACGG
CTGGAAATATTTTGTTTGGTAGAATTATTAAAAGATATTTTAAGAAATG
AGAATCTGATCTCGTTGTCAACTTTTTATTTGTTTTGGTGATTGGAGTA
GGCAATATAAGAATAGAGGTGAAAAGGACAAGAAAATGGTTGAGGACAC
GAAGTGGAAGGTTCTGGTGAGAAGAGTAGCGTCTGCTAAAGCCATGTCT
TCGTTGTCTCAATCAAAGAAGAAAAAAAGTGAAGTGTTATTTAAAGAAG
AAGCAGAAGATTTGATCAGAATCCGGAACAAGTTAATGTGAATATACAA
TATATGTGTTTTTATTAGATTATATAGTGAGCGATTGTTAGAAGCCATT
AATTACTTGAGTCGGAAGGTTTATTTGTTTGTTATTTGTCTCTCGGACT
GGTTGATTGGGTTAAACTTGTCAATGAGTAACCCTTTGAATCTGTCGTT
TTGTTTGGTCGGTCGAATTATATAGAAATGTATTATTGGTTATAAAATG
GTATTAGGGTTTTTGGAG

SEQ ID NO:6 shows the reverse complement of the exemplary BT2 polynucleotide of SEQ ID NO:3.

CAAACTACCAAACCTACAGAAATCTCTCTTCTTCTTCTTCTCTACAAAA
TTTCAATGGAAGCTGTTCTTGTCGCAATGTCCGTCCCCGCCACAACGGA
AGACGACGGATTTTCGTTAATCACCGATAAACTTTCATATAATCTAACG
CCGACGTCGGACGTTGAGATCGTTACCTCCGATAACCGTCGGATTCCGG
CACACTCCGGCGTTCTGGTAAGTTGTTATTTATTTTGGTTTACGGTTTA
CATTTTTCTGGTTTGGTTTTTAGCTAAAATTTGTGGATAATGCAGGCTT
CAGCTTCACCGGTACTGATGAACATCATGAAGAAACCGATGAGACGTTA
CAGAGGCTGTGGATCTAAAAGAGTCATCAAGATTCTTGGCGTTCCATGC
GACGCCGTTTCGGTTTTTATCAAATTCCTCTACTCTTCTAGGTTGGTTT
GGTTCGGTTATTCTTCTTGAATTTTCCAAATGTAGAAACTTCAAACCGA
TGGTTATTTCCGGTACTACACAATTTTCCCGGTTTTTCTTTTTCATACA
CGGATTTGAAGAAACTCTGTTTTTTTTTGGGCTATTTTCTTGGGGAAAC
AATAGCTGTCAGGAAATGACTAAACTACCCTTTTGCGTCTCTGTTTTAG
TTTGACGGAGGATGAGATGGAGAGATATGGAATCCATCTTTTAGCGTTG
TCTCACGTCTACATGGTCACCCAGCTGAAGCAACGGTGCTCAAAAGGCG
TTGTACAACGTTTAACTACAGAGAACGTAGTCGACGTCCTCCAGCTAGC
TCGGCTCTGCGACGCACCCGATGTTTGCCTTCGATCTATGCGTCTGATC
CATTCGCAGTTTAAGACCGTTGAGCAGACTGAAGGATGGAAGTTTATTC
AAGAACATGATCCTTTCCTCGAGCTCGACATTCTCCAGTTCATCGATGA
CGCCGAATCGGTAAACTTACTTATATGTTTTGAAAACACAGCATCTCTG
TTTTTCTTAGTACCTTCGATAACACATTTTGTTCAGCTAGGTTTAGTTT
TTGTTCCGCGCTAGTAAAAAGATAATTTTTTTTTTTTTAAATTCAGTTT
ATTTAGTTTGGCATGTTGCACAAAGTTTAGAGTATTAGTTAACTCATGT
TTGATTAGATTTGGTTTATATACCTAATTAACCCCAAAACATTTGGTCA
TTTTTCTGATTATGTGTTGATTTATAATTTAACGCAGAGGAAGAAAAGA
AGGCGACGACATAGAAAGGAGCAGGATCTTTACATGCAACTAAGCGAGG
CAATGGAGTGCATAGAGCACATATGCACACAAGGTTGTACATTGGTCGG
TCCATCAAACGTAGTAGATAACAACAAAAAGTCAATGACTGCGGAAAAG
TCAGAGCCTTGTAAGGCGTTTTCCACGTGTTATGGTCTTCAGCTTTTGA
TACGTCACTTTGCCGTATGCAAGAGGAGGAATAACGATAAGGGTTGTCT
TCGTTGCAAGCGGATGCTTCAACTCTTTAGACTCCATTCTTTGATTTGT
GATCAACCCGATTCTTGTCGCGTCCCTCTCTGCAGGTATGTCCCAACTT
GTTTTGTTAGGAATTTCACATTTGAAATAAATCAAAAAGTATGAGCGAA
TCCATTTACGCTTAATTAGTAAGTTTGAAATTGATGATTTTTAGTATGT
TTATGAATAAAAACCAATGTTTTGTTGAAAGTGAAAAAATTTCATTTTT
GGTAAGAGACAAGGTTTGTTTATCTTTTTGTGTAGACCCACCTTTAGAA
AAGTTGCAAATTGCTATGTGAATTGATGATGGGTCGTAATCCGACGGCT
GCAAATTGTTGCCATTTCTTGAAATTTGTAGATATTTTAATAGAATCTC
ATGTGTTCTTTTCAACTCTTTTTTTTGGGACTAGGCAATTTAGGAAACG
GGGAGAACAAGACAAGAAAATGGGTGAAGACACCAAGTGGAAGCTTCTG
GTGACAAGGGTTGTGTCTGCAAAAGCTATGACGTCGTTGTGTCAGTCAA
AGAAGAACAAATGTGAACAAGCACAAGGGGTTTAATCAAAACCAGGAAG
GAGTTAGTTAGGTTTGAAGAAGATTAAGTAAGAAGCTTTCTTATAATTT
GATATCCATTCTTTTTGTAGTATAATAGTATTTGTTAGTTCGATTCTTT
ATTAGAGGGGTTTAGTTTGTATAGGAGTAAACCCTTTTAAATTGGCTGT
ATAAGTAGGTCATATTCAATTGTTTTGTTCTATTTATCTGAAAAATGTC
AGCATGTAATACAAAACAAATAAATGTTCAAATTAAAGGGAGGATTACA
AGAGGTGTTTTTAATGTTATTGACTTTGGCC

SEQ ID NO:7 shows an exemplary bt1 polynucleotide encoding a mutant BTB1 polypeptide:

CCATCATCAAATTAAAACCCTCCAGCCTCCAACAACAAAATTTACCAAC
ACTCTCTCTTTCTCTTTCTCTCTGAAAACTTAAAACGATGGCTATAACC
GCCACTCAAAACGACGGCGTTTCACTAAACGCCAATAAGATTTCGTATG
ATCTTGTGGAAACTGATGTTGAGATCATCACCTCCGGTCGTCGTAGTAT
TCCGGCACACTCCGGCATCCTCGTAAGTTTCTTTTCTTCTTACTTTCTT
TGTATGATCGAATATTTTTTTATTTTGGTGCTTATCTTTTTTGTTTGTT
TAAAATTGTTAAATGCAGGCTTCGGTCTCACCGGTACTGACGAACATCA
TCGAGAAGCCGAGGAAGATTCACGGCGGATCATCGAAGAAAGTTATTAA
GATTCTCGGTGTTCCATGCGACGCCGTTTCAGTCTTCGTCAGATTCCTC
TATTCTCCGAGGTTCGTCTTTCTCTCTTTTTCATATCTTCTTGATCGTC
AGATTTTTTTTTTTAATCCGTAGATTTATTACTTCAAAAATACAAATCA
TACTAAAAAGAATGCTTGATTAAATACATCATCTTAAAGATCTTTACCA
AACTCGACTTTTCCAACGTTTTAATCATACATTAAAATCACTACAACCA
GATTGCGATTGATTTAGACTTGTTTCATATTCAAAAACGTGTGAAACAC
AACACAAGGAACACGAAACTAGTACCAACATCAAATTTGAATAAATCAA
CGGCAAATTTTACCCTTAGTATGCAAAATGACTAGAATACCCCTATATG
TATCTTTTGTTTAGTGTCACGGAGAATGAGATGGAAAAATACGGAATCC
ATCTACTGGCTTTATCACACGTGTATATGGTGACTCAGTTAAAGCAACG
GTGCACTAAAGGCGTCGGTGAGAGAGTAACCGCCTTTACCGACCGTTAC
CGACCGTTTTCATCCCTACTTGTAACTGAAATAAAAACAGAGTTTTCAT
AGACTTCAGAACTAAATCAACCAAAACCAAATAAGCTGAAAAATAAAAT
AAAAAACGAATGCATAGCAAAAACTGGTATTAAGCTTTGAATTCTAAAC
TATAAGACAAGTTTTGTAGTTTACCGCGGGATTGTCCAAGCGATCAAGC
TCGTGGCCATCGGGGATACTCCCAGAGATATACTGCCAGCCAAGGATGT
CACATTCCAAATCAGCATCCAAAAGTGTATCCCAAAAATACTTCATTCC
CCATTTCCATGGAAGGAGAAAAAACTTCACAGCAAAGCTTGAAACAATC
ACTCTTATTCTGTTATGCATCCATCCGGTAGCCCAAAGCTCTCTCATTC
CGGCATCCACCAACGGATAACCGGTCCTGCCTTGTCTCCAGGCCTTGAA
CTTATCAACATCAGCATCCCAAGGGAAAAACCGAAAATGACTCAACAAC
GATTGCTCGTGAGTAAACGGGAAGTTGAAACATATATACCGAGAATACT
CTCTTAAACCGATTCCCCTAAGAAAAAGATCTGCACTTTCTTCTCCTTC
ACTGTTCCACTGGTCGGACCATCGTCTAACTTAGACAACAAGTCAACAT
GTCAAGCAAAACCCGGTCCATGCAGTGCGTTTTCGACTTGTTACGGACT
TCAACTTCTTATACGTCACTTTGCAGTATGCAAGAAAAGAGTCGATGGC
AAAGGTTGTGTCCGATGCAAGAGAATGATTCAACTCCTTAGACTCCATT
CTTCGATTTGTGACCAATCTGAATCTTGCCGTGTCCCTCTTTGCAGGTA
AAAATCCGACATTTTAGAATAATCAAAACCACATTTTAAATGTTTCTAA
TGATAAAAGTTTTTCACTTTTCGATAGTAGATCTCATTTGATTTTTTTT
TAACTTTCGGCGCCAATAATAATTATCTTTTTGTGGTGGTAGCCTACTT
ATTAGATGATGAGTCGTGATCGGACGGCTGGAAATATTTTGTTTGGTAG
AATTATTAAAAGATATTTTAAGAAATGAGAATCTGATCTCGTTGTCAAC
TTTTTATTTGTTTTGGTGATTGGAGTAGGCAATATAAGAATAGAGGTGA
AAAGGACAAGAAAATGGTTGAGGACACGAAGTGGAAGGTTCTGGTGAGA
AGAGTAGCGTCTGCTAAAGCCATGTCTTCGTTGTCTCAATCAAAGAAGA
AAAAAAGTGAAGTGTTATTTAAAGAAGAAGCAGAAGATTTGATCAGAAT
CCGGAACAAGTTAATGTGAATATACAATATATGTGTTTTTATTAGATTA
TATAGTGAGCGATTGTTAGAAGCCATTAATTACTTGAGTCGGAAGGTTT
ATTTGTTTGTTATTTGTCTCTCGGACTGGTTGATTGGGTTAAACTTGTC
AATGAGTAACCCTTTGAATCTGTCGTTTTGTTTGGTCGGTCGAATTATA
TAGAAATGTATTATTGGTTATAAAATGGTATTAGGGTTTTTGGAG

SEQ ID NO:8 shows an exemplary mutant BTB1 polypeptide:

### MAITATQNDGVSLNANKISYDLVETDVEIITSGRR

SEQ ID NO:9 shows an exemplary bt2 polynucleotide encoding a mutant BTB2 polypeptide:

CAAACTACCAAACCTACAGAAATCTCTCTTCTTCTTCTTCTCTACAAAATTTCAATGGAAGCTGTT
CTTGTCGCAATGTCCGTCCCCGCCACAACGGAAGACGACGGATTTTCGTTAATCACCGATAAACTT
TCATATAATCTAACGCCGACGTCGGACGTTGAGATCGTTACCTCCGATAACCGTCGGATTCCGGCA
CACTCCGGCGTTCTGGTAAGTTGTTATTTATTTTGGTTTACGGTTTACATTTTTCTGGTTTGGTTT
TTAGCTAAAATTTGTGGATAATGCAGGCTTCAGCTTCACCGGTACTGATGAACATCATGAAGAAAC
CGATGAGACGTTACAGAGGCTGTGGATCTAAAAGAGTCATCAAGATTCTTGGCGTTCCATGCGACG
CCGTTTCGGTTTTTATCAAATTCCTCTACTCTTCTAGGTTGGTTTGGTTCGGTTATTCTTCTTGAA
TTTTCCAAATGTAGAAACTTCAAACCGATGGTTATTTCCGGTACTACACAATTTTCCCGGTTTTTC
TTTTTCATACACGGATTTGAAGAAACTCTGTTTTTTTTTGGGCTATTTTCTTGGGGAAACAATAGC
TGTCAGGAAATGACTAAACTACCCTTTTGCGTCTCTGTTTTAGTTTGACGGAGGATGAGATGGAGA
GATATGGAATCCATCTTTTAGCGTTGTCTCACGTCTACATGGTCACCCAGCTGAAGCAACGGTGCT
CAAAAGGCGTTGTACAACGTTTAACTACAGAGAACGTAGTCGACGTCCTCCAGCTAGCTCGGCTCT
GCGACGCACCCGATGTTTGCCTTCGATCTATGCGTCTGATCCATTCGCAGTTTAAGACCGTTGAGC
AGACTGAAGGATGGAAGTTTATTCAAGAACATGATCCTTTCCTCGAGCTCGACATTCTCCAGTTCA
TCGATGACGCCGAATCGGTAAACTTACTTATATGTTTTGAAAACACAGCATCTCTGTTTTTCTTAG
TACCTTCGATAACACATTTTGTTCAGCTAGGTTTAGTTTTTGTTCCGCGCTAGTAAAAAGATAATT
TTTTTTTTTTTAAATTCAGTTTATTTAGTTTGGCATGTTGCACAAAGTTTAGAGTATTAGTTAACT
CATGTTTGATTAGATTTGGTTTATATACCTAATTAACCCCAAAACATTTGGTCATTTTTCTGATTA
TGTGTTGATTTATAATTTAACGCAGAGGAAGAAAAGAAGGCGACGACATAGAAAGGAGCAGGATCT
TTACATGCAACTAAGCGAGGCAATGGAGTGCATAGAGCACATATGCACACAAGGTTGTACATTGGT
CGGTCCATCAAACGTAGTAGATAACAACAAAAAGTCAATGACTGCGGAAAAGTCAGAGCCTTGTAA
GGCGTTTTCCACGTGTTATGGTCTTCAGCTTTTGATACCTGATGGGCTGCCTGTATCGAGTGGTGA
TTTTGTGCCGAGCTGCCGGTCGGGGAGCTGTTGGCTGGCTGGTGGCAGGATATATTGTGGTGTAAA
CAAATTGACGCTTAGACAACTTAATAACACATTGCGGACGTTTTTAATGTACTGGGGTGGTTTTTC
TTTTCACCAGTGAGACGGGCAACAGCTGATTGCCCTTCACCGCCTGGCCCTGAGAGAGTTGCAGCA
AGCGGTCCACGCTGGTTTGCCCCAGCAGGCGAAAATCCTGTTTGATGGTGGTTCCGAAATCGGCAA
AATCCCTTATAAATCAAAAGAATAGCCCGAGATAGGGTTGAGTGTTGTTCCAGTTTGGAACAAGAG
TCCACTATTAAAGAACGTGGACTCCAACGTCAAAGGGCGAAAAACCGTCTATCAGGGCGATGGCCC
ACTACGTGAACCATCACCCAAATCAAGTTTTTTGGGGTCGAGGTGCCGTAAAGCACTAAATCGGAA
CCCTAAAGGGAGCCCCCGATTTAGAGCTTGACGGGGAAAGCCGGCGAACGTGGCGAGAAAGGAAGG
GAAGAAAGCGAAAGGAGCGGGCGCCATTCAGGCTGCGCAACTGTTGGGAAGGGCGATCGGTGCGGG
CCTCTTCGCTATTACGCCAGCTGGCGAAAGGGGGATGTGCTGCAAGGCGATTAAGTTGGGTAACGC
CAGGGTTTTCCCAGTCACGACGTTGTAAAACGACGGCCAGTGAATTCCCGATCTAGTAACATAGAT
GACACCGCGCGCGATAATTTATCCTAGTTTGCGCGCTATATTTTGTTTTCTATCGCGTATTAAATG
TATAATTGCGGGACTCTAATCATAAAAACCCATCTCATAAATAACGTCATGCATTACATGTTAATT
ATTACATGCTTAACGTAATTCAACAGAAATTATATGATAATCATCGCAAGACCGGCAACAGGATTC
AATCTTAAGAAACTTTATTGCCAAATGTTTGAACGATCGGGGAAATTCGAGCTCGGTACCCGGGGA
TCCTCTAGAGTCCCCCGTGTTCTCTCCAAATGAAATGAACTTCCTTATATAGAGGAAGGGTCTTGC
GAAGGATAGTGGGATTGTGCGTCATCCCTTACGTCAGTGGAGATATCACATCAATCCACTTGCTTT
GAAGACGTGGTTGGAACGTCTTCTTTTTCCACGATGCTCCTCGTGGGTGGGGGTCCATCTTTGGGA
CCACTGTCGGCAGAGGCATCTTCAACGATGGCCTTTCCTTTATCGCAATGATGGCATTTGTAGGAG
CCACCTTCCTTTTCCACTATCTTCACAATAAAGTGACAGATAGCTGGGCAATGGAATCCGAGGAGG
TTTCCGGATATTACCCTTTGTTGAAAAGTCTCAATTGCCCTTTGGTCTTCTGAGACTGTATCTTTG
ATATTTTTGGAGTAGACAAGTGTGTCGTGCTCCACCATGTTGACGAAGATTTTCTTCTTGTCATTG
AGTCGTAAGAGACTCTGTATGAACTGTTCGCCAGTCTTTACGGCGAGTTCTGTTAGGTCCTCTATT
TGAATCTTTGACTCCATGGCCTTTGATTCAGTGGGAACTACCTTTTTAGAGACTCCAATCTCTATT
ACTTGCCTTGGTTTGTGAAGCAAGCCTTGAATCGTCCATACTGGAATAGTACTTCTGATCTTGAGA
AATATATCTTTCTCTGTGTTCTTGATGCAGTTAGTCCTGAATCTTTTGACTGCATCTTTAACCTTC
TTGGGAAGGTATTTGATTTCCTGGAGATTATTGCTCGGGTAGATCGTCTTGATGAGACCTGCTGCG
TAAGCCTCTCTAACCATCTGTGGGTTAGCATTCTTTCTGAAATTGAAAAGGCTAATCTGGGGACCT
GCAGGCATGCAAGCTTGGCGTAATCATGGTCATAGCTGTTTCCTGTGTGAAATTGTTATCCGCTCA
CAATTCCACACAACATACGAGCCGGAAGCATAAAGTGTAAAGCCTGGGGTGCCTAATGAGTGAGCT
AACTCACATTAATTGCGTTGCGCTCACTGCCCGCTTTCCAGTCGGGAAACCTGTCGTGCCAGCTGC
ATTAATGAATCGGCCAACGCGCGGGGAGAGGCGGTTTGCGTATTGGGCCAAAGACAAAAGGGCGAC
ATTCAACCGATTGAGGGAGGGAAGGTAAATATTGACGGAAATTATTCATTAAAGGTGAATTATCAC
CGTCACCGACTTGAGCCATTTGGGAATTAGAGCCAGCAAAATCACCAGTAGCACCATTACCATTAG
CAAGGCCGGAAACGTCACCAATGAAACCATCGATAGCAGCACCGTAATCAGTAGCGACAGAATCAA
GTTTGCCTTTAGCGTCAGACTGTAGCGCGTTTTCATCGGCATTTTCGGTCATAGCCCCCTTATTAG
CGTTTGCCATCTTTTCATAATCAAAATCACCGGAACCAGAGCCACCACCGGAACCGCCTCCCTCAG
AGCCGCCACCCTCAGAACCGCCACCCTCAGAGCCACCACCCTCAGAGCCGCCACCAGAACCACCAC
CAGAGCCGCCGCCAGCATTGACAGGAGGCCCGATCTAGTAACATAGATGACACCGCGCGCGATAAT
TTATCCTAGTTTGCGCGCTATATTTTGTTTTCTATCGCGTATTAAATGTATAATTGCGGGACTCTA
ATCATAAAAACCCATCTCATAAATAACGTCATGCATTACATGTTAATTATTACATGCTTAACGTAA
TTCAACAGAAATTATATGATAATCATCGCAAGACCGGCAACAGGATTCAATCTTAAGAAACTTTAT
TGCCAAATGTTTGAACGATCGGGGATCATCCGGGTCTGTGGCGGGAACTCCACGAAAATATCCGAA
CGCAGCAAGATATCGCGGTGCATCTCGGTCTTGCCTGGGCAGTCGCCGCCGACGCCGTTGATGTGG
ACGCCGGGCCCGATCATATTGTCGCTCAGGATCGTGGCGTTGTGCTTGTCGGCCGTTGCTGTCGTA
ATGATATCGGCACCTTCGACCGCCTGTTCCGCAGAGATCCCGTGGGCGAAGAACTCCAGCATGAGA
TCCCCGCGCTGGAGGATCATCCAGCCGGCGTCCCGGAAAACGATTCCGAAGCCCAACCTTTCATAG
AAGGCGGCGGTGGAATCGAAATCTCGTGATGGCAGGTTGGGCGTCGCTTGGTCGGTCATTTCGAAC
CCCAGAGTCCCGCTCAGAAGAACTCGTCAAGAAGGCGATAGAAGGCGATGCGCTGCGAATCGGGAG
CGGCGATACCGTAAAGCACGAGGAAGCGGTCAGCCCATTCGCCGCCAAGCTCTTCAGCAATATCAC
GGGTAGCCAACGCTATGTCCTGATAGCGGTCCGCCACACCCAGCCGGCCACAGTCGATGAATCCAG
AAAAGCGGCCATTTTCCACCATGATATTCGGCAAGCAGGCATCGCCATGGGTCACGACGAGATCAT
CGCCGTCGGGCATGCGCGCCTTGAGCCTGGCGAACAGTTCGGCTGGCGCGAGCCCCTGATGCTCTT
CGTCCAGATCATCCTGATCGACAAGACCGGCTTCCATCCGAGTACGTGCTCGCTCGATGCGATGTT
TCGCTTGGTGGTCGAATGGGCAGGTAGCCGGATCAAGCGTATGCAGCCGCCGCATTGCATCAGCCA
TGATGGATACTTTCTCGGCAGGAGCAAGGTGAGATGACAGGAGATCCTGCCCCGGCACTTCGCCCA
ATAGCAGCCAGTCCCTTCCCGCTTCAGTGACAACGTCGAGCACAGCTGCGCAAGGAACGCCCGTCG
TGGCCAGCCACGATAGCCGCGCTGCCTCGTCCTGCAGTTCATTCAGGGCACCGGACAGGTCGGTCT
TGACAAAAAGAACCGGGCGCCCCTGCGCTGACAGCCGGAACACGGCGGCATCAGAGCAGCCGATTG
TCTGTTGTGCCCAGTCATAGCCGAATAGCCTCTCCACCCAAGCGGCCGGAGAACCTGCGTGCAATC
CATCTTGTTCAATCATGCGAAACGATCCAGATCCGGTGCAGATTATTTGGATTGAGAGTGAATATG
AGACTCTAATTGGATACCGAGGGGAATTTATGGAACGTCAGTGGAGCATTTTTGACAAGAAATATT
TGCTAGCTGATAGTGACCTTAGGCGACTTTTGAACGCGCAATAATGGTTTCTGACGTATGTGCTTA
GCTCATTAAACTCCAGAAACCCGCGGCTGAGTGGCTCCTTCAACGTTGCGGTTCTGTCAGTTCCAA
ACGTAAAACGGCTTGTCCCGCGTCATCGGCGGGGGTCATAACGTGACTCCCTTAATTCTCCGCTCA
TGATCAGATTGTCGTTTCCCGCCTTCAGTTTAAACTATCAGTGTTTGACAGGATATATTGGCGGGT
AAACCTAAGAGAAAAGAGCGTTTATTAGAATAATCGGATATTTAAAAGGGCGTGAAAAGGTTTATC
CGTTCGTCCATTTGTATGTGCATGCCAACCACAGGTCTTGAAATTTGTAGATATTTTAATAGAATC
TCATGTGTTCTTTTCAACTCTTTTTTTTGGGACTAGGCAATTTAGGAAACGGGGAGAACAAGACAA
GAAAATGGGTGAAGACACCAAGTGGAAGCTTCTGGTGACAAGGGTTGTGTCTGCAAAAGCTATGAC
GTCGTTGTGTCAGTCAAAGAAGAACAAATGTGAACAAGCACAAGGGGTTTAATCAAAACCAGGAAG
GAGTTAGTTAGGTTTGAAGAAGATTAAGTAAGAAGCTTTCTTATAATTTGATATCCATTCTTTTTG
TAGTATAATAGTATTTGTTAGTTCGATTCTTTATTAGAGGGGTTTAGTTTGTATAGGAGTAAACCC
TTTTAAATTGGCTGTATAAGTAGGTCATATTCAATTGTTTTGTTCTATTTATCTGAAAAATGTCAG
CATGTAATACAAAACAAATAAATGTTCAAATTAAAGGGAGGATTACAAGAGGTGTTTTTAATGTTA
TTGACTTTGGCC

SEQ ID NO:10 shows an exemplary mutant BTB2 polypeptide:

MEAVLVAMSVPATTEDDGFSLITDKLSYNLTPTSDVEIVTSDNRRIPAH
SGVLVSCYLFWFTVYIFLVWFL

SEQ ID NO: 11 shows a further exemplary polynucleotide encoding a BTB1 polypeptide (Oryza saliva Os01g68020):

CCCTCAGATGCATAGTACTCGCACGCCGCGACGCCTCTGCTGAACATTTCGCTCTCTTCCAAGA
TATTTTTTCCTCCCACGATTCCTTCCTCCCATCCCTGTGGGGATTCATCGGCTCATCCGTACTA
GCACAAGATGTGCGAGGGTGTGCGGGCCGCCGGCGACGCCGCCGCCGCCGCCGACGTCGACGTC
ATCACCTCCTCCGGCCGCCGGAGGATCCCCGCGCATTCCACTGTTCTTGTAAGCTTCTCCTCTT
TCATCAGCAAATTCATGAATCCTTTTAGCTGGAGATAGTTTCTGTGGTCAAATTCTGGTTGGCG
TCGGGCGTGTTCTGACGAGGAAATCTCCGTTTAATTCTTTGTTTGTTTGGTTTGATCACCAGGC
GTCGGCGTCGCCGGTGCTGGAGAGCATCCTGCAGCGCCGCCTGAAGAAGGAGAGGGACGCCGCC
GCCGGCGGCGGCAAGGTCCGCAGGGCCGTCGTCCTGATCCGCGGCGTCACCGACGACGCCGCGG
CCGCCTTCGTCCGCCTCCTCTACGCCGGCAGCAGGTACCGTGCCCTCGTCCATTCCATTCGGCC
ATTCCCCTCTAGGATGGGATTTCTGCTGGTAGTCTTTGCTTTTCGAGCATCCGTTTCTTGCAAA
AGATGGGCGATTAGTTGAAGAATTAATGGTCTTCGAAGACTAATTACTCGTATTAACTCACGAG
GGGGAAAGAAAAACATGTCACACGTGTAGGCCACGCTGACAAGAAACTCGATCTCTTCTTCTAT
GGTTTAGTTGAAAATTATATATTTTTAGTTTATTTTTTTATTAGAGGGTACGTTTTTTCTGTTG
ATCATGGTAGCAGTAATTGGCTACTAATAATTGCTACCTAAACTATGAAAACGGTAGATACGAC
GAAATGATGTTGAAAGTTTTTAAGCGATGATAAACAGTGGGTGAAAATATGCTTAATCACTATT
CTGTATCTGTAGCAGAACGAGCAAAACCTTCGGAGCGAATGCTGTAGGAAAAAAACTGAAAAAA
AAAATCTGGTGTCCAAAGTCCTTGCTAGATAGGCAGTCCAATCAAGGCAGGCCCGGCTAAACCA
CGGATATTTTCTTCATCTATCACACAGCTTACAGATCCAATGAACCATTCCAAGAATCTCGCTA
AGTGATTAGACAGATCGCTTTGGCAGCCTCCGTGATAAAATCCCCTCCAAGTTGGCCAATTTGC
CGATTTTTTGTGCCTCATCAAACTGTGCGAGTCTTGACAAGGCAGGTCAGGTAAGGTGACACAA
CACAACCCCACACAAATGACACAAACAGAGCATCGAAACATCGCCATCTCCAGCTCCGAGAGCC
GTAAACAAATCTCGTGGAGCAGAATCCATGCCTTGCCTCGGCGCGTCGACTTGGACGGCACAGA
CAGAGAAAAAAAAAGAGAGAAAAAGTAGTAGTAAAGCGAAGCAACGATAAGTACTAGACCACTA
GAGCAGTAGGAGAAGTCGAGAAGAGGATGATTTTGGCTTTCTTTTTCCGCCGCTTGGTTTGGAG
TTTGGTGTGGTAGCGCCTGCCTAGTTGGAACCCTCCCCTTGCAGGCGAGCACCAAGCAGGCAGC
CGATCTCGTCGGCGCCACAAGAAAAATCGTTGCTGTCGCTGGTGCGGCTTGCAGGGCCGGGACC
GCTCGCCGCCTCAGCCATCATCTCGTGTTGGTTTTTGTTGAAAAAAAAAAAGAACTCTCTTTTT
GTGTGTGGAGATGCAGAGGTAGGGACCTGCCCAAACTGTCCCAGTGTCCCGATCCGTTTGAAAC
GAATGAAGACTTTCGTGCCTTTCTCAACATTAGTAATAGAGTAAATTTCACAAAAGTAGAAGTT
TTATGGTTCAAGTTGCAGAAAATCACACACATTTTGACACTTGACACTTGACACTTAAGTACAT
ATATTTCGGTAGTTTAGTTTCATAAAACCACACTATCGATGAATGGATTCACCCGTGAGATGAT
ATGGTTGTTCCACCCGTGAGATGACGTGGTTGTTCCATCTAAGATGAGGACGTGACATCATATT
AATTTCGTGCGATGGCTGGTAATAGAATGCCATGTCCTCGTCTTAGGTGTAATAGCCACATCAT
CTCACGGGTGAATCTATACATCGATAATGTAGTTTTGTGAAACTACACAACCAAAATATATGTA
CTCAAGTGCCAAGTGTCAAAGTGTATGTGATTTTCTGTAACTTGGACCACAAAACGTGTAGTAG
AAATATTTGAGAAAAAAACATTTGCATATTAGACGTGCTAATACTGTGTAAATTTTGTTAACTA
ATTGTGCAGTGGTGATGAGGAGGAGATTGACGAGAAGAGCGCGGCGCAGATGCTGGTGCTGGCG
CACGCGTACCGGGTGCCGTGGCTGAAGCGGCGGTGCGAGGGGGCGATCGGGTCGCGGCTCACGG
CGGAGTCGGTGGTGGACACGATGCAGCTGGCGGCGCTGTGCGACGCGCCGCAGCTGCACCTGCG
CTGCACCAGGCTGCTCGCCAAGGAGTTCAAGGCCGTGGAGAAGACCGAGGCGTGGCGATTCCTC
CAGGAGAACGACCCCTGGCTCGAGCTCGACATCCTCCAGCGCCTCCACGACGCCGACCTGGTAT
ACATCATTATCGCTGTTCATCAACATGGTAGAATTTTACAGGAATTTCACACAAAAAAAATTCA
AACTTTGAATTGTGCACTCGATGTGGTGTGGGGTATCCATGTTGGGCGCTTTTGTAAATGGCTC
TGTGACACACGGTTCCATTGACACCGAAATGCATGGGAGGATTTGCACAGTGGGTCGTGCACTA
GGGGAACATGATTTTGAGCGTGTTTTACTGGGATCTTTCGAGCCTTAAGTCCCTGGAATAGCCG
GGGGCCACCGTGTCGCACTCGTGCACACATTTTATGTCGAGACGTGCTTTGCGTTTTGTGGTTG
GTTGGTGGATTGAATGAGAAATTTCTTGTATCTGGGGGAGGCTAGGTCGGTCCGGTTGGTCTGT
CCAACCACGTTGAATGGACTTCGAACGGCTTACCGTCTCGAAGTTTCCCGATCCTAAACGTTTT
TGTTATGGTCGAATGATTTTGATTTTGACAAGAACCTAATTAGGACCTGTTTACTTTGATGTCA
TTTTCAATCTTACCAAATTTTGGTAAAGTTGTCAAAATTGTGGCTACATTTAGTTTGCTGTCAA
ATTTTGATAACTATATAAGAAATCCTGCCAATTACCAAAATTTTGACAACTATGCCAAAATTTT
GGTAATGTTTTTTTCTTTTGCATTAAAGTGAACAGGCCCTTAATTGACCAATAATCTCTGAAAT
AGACTGATAAAATCGCATGATTTTTGTTTTAGTATTTTTTTTACTACTACTAGAATTCATGGTG
GTTGATTTTTTTTTTCTCCGGTGTGATTGAATGGTGCAGCGGCGGAGGAAGTGGAGGAGGAAGA
GGGCGGAGCAAGGGGTCTACGTGGAGCTGAGCGAGGCGATGGACTGCCTGAGCCACATCTGCAC
GGAGGGGTGCACGGAGGTCGGGCCGGTGGGGCGTGCGCCGGCGGCGGCGCCGTGCCCGGCGTAC
GCGACGGCGTGCCGGGGACTGCAGCTGCTGATCCGCCACTTCTCCCGGTGCCACCGCACCAGCT
GCCCGCGCTGCCAGCGCATGTGGCAGCTGCTCCGCCTCCACGCCGCCCTCTGCGACCTCCCCGA
CGGCCACTGCAACACCCCTCTCTGCATGTGAGTGATCACAATGTCACACTCACACACCCATAAA
TGCGTCTTTTAAACATACGCTTGAAAAGATGAAAACTAGCGATATCATCTAATCTCACTAAAAT
ATCGTTGAAAGTCTAAATGCGTATGCGTAATATTCAATTAGCTTACATTGATCTGGTTTTGACT
TGGGAAAAAAAAATCTTTGCATTGTGTATTGCAGGCAGTTCAGGAGAAAGGAGGAGGAGAAGGC
GGCAGCGAAGGCGAAGGCAAAGGCCGGCGATGATGACGATAAGTGGGGGCTTCTGGTGAAGAAG
GTGAGGGTTGCCAGGGCCATGTCTTCTCTGGGCAAGAGGAGGCAGATGAGTTGCTCCCAGTGCT
GAGCAAGTGCAATCACAGCATCAGCCTGAGACAGTCAAGAGAAGAGTATTGTAGTATAGAAGGG
TACTACTATGTAGTTAGGCATAGTGCATGAGTGTATTGAAGTGAGCTATTTTTTGGTTTGTTCA
AGATGGTTTGGTCTCTCAAGTTTGAGTTGCTTTACTGTCTTCAATTTGTAGAGTTCTTTTGTGA
GGTGAGAGAAATGGGCTGAAATCTGTTGTAAAATTGCCTATATCGTAAATAAACTTTTAATAGT
GAGGTATATGATCATTACAGTTTCATACCTGGATGATTACATTGATAGA

SEQ ID NO:12 shows a further exemplary BTB1 polypeptide (Oryza saliva Os01g68020):

MCEGVRAAGDAAAAADVDVITSSGRRRIPAHSTVLASASPVLESILQRR
LKKERDAAAGGGKVRRAVVLIRGVTDDAAAAFVRLLYAGSSGDEEEIDE
KSAAQMLVLAHAYRVPWLKRRCEGAIGSRLTAESVVDTMQLAALCDAPQ
LHLRCTRLLAKEFKAVEKTEAWRFLQENDPWLELDILQRLHDADLRRRK
WRRKRAEQGVYVELSEAMDCLSHICTEGCTEVGPVGRAPAAAPCPAYAT
ACRGLQLLIRHFSRCHRTSCPRCQRMWQLLRLHAALCDLPDGHCNTPLC
MQFRRKEEEKAAAKAKAKAGDDDDKWGLLVKKVRVARAMSSLGKRRQMS
CSQC

SEQ ID NO:13 shows a further exemplary polynucleotide encoding a BTB1 polypeptide (Zea mays GRMZM2G004161):

AATTGGCCTGGCTACCCAGACAAGAGCACGCAACGCAAGCAACTGCGACGTCTCCCGCCCCCGC
CGCGCCCCGGACCTTTTCCGCAGATATTTTTTTTATCCCCCCACGCCCCCGGCTCGTTCCCTTC
CATTCCACCACCTCTCGCCAGACAACGAGATGTGCGAGGCACCGCGGCTCGGCAACCGCGCCGG
CGCCGGCGACCCGGCCGCCGCCGACGTCGACGTCGTCACGACGGGCGGGCGCCGGAGGATCCCC
GCGCATTCCTCTGTCCTGGTAAGCGTCCGATCCCTTTTTCCCCCCCTCCTTGGCGCATTGCTCC
GTTCGGCCGTCCCGTCCGGGTCGTCTAGCAGTCGTCTGACGGGAGCTGTGTCCGTCTCTGTTTC
CGCGCGCGCTAGGCCTCGGCGTCGCCGGTGCTCGGCAGCATCCTGGAGCGTCGCCTGCGGAAGG
ACAGGGAGAGCGGCAAGCCCGGGCGGTCCGTCGTCCGGATCCGCGGCGTCACCGACGACGCCGC
CGCGGCCTTCGTCCGCCTCCTCTACGCCGGCAGGTAACGCCCGTGGGCACCGTGCGTGCCTGTC
TGTCTCCCTTTCAATTCCCCGGTCGCGCGCGCTCAGCCATGGCGGCCTCCTTGTTGCTAGTCTT
GTTTTCGCACATGTTAGTTCCTCGCGAGGCTGCGGCCGCCTGGTCCCCGGCCTGGGGCTGGACA
AGCCAGTGCGGCACACGATTATTAGCAAGGACTAATCCTGCGTTATTCTAAACGCACGAGGAGG
GGAAACATGGCGCGCGTGTGTGCTCCACCCACACGTTAACTGCCCGGAAGATCAACCTTTATTC
TTAGACATCATCCAGAAGCGTTTTTATATTGAAAAAAAGTATTCTAGTACTTTTTTATTCTTTA
TTTATCTCGAGTCCATCTGTACGTTTCCTTCTTTAACAGTATATACACACAGTATAAACCTGAA
ATTCGAGGTGTCCAACGTTCTAGGAATGGCAAGAAGCAAGTCCATGACCACGCGCTAGCTTCAA
CAATCGATTTTAAAACTTTGAGTAGGTACCAAATCCAACCAAACCGATCGAGCTCCCCCGTAGT
AGGAAAACCTTGCACCCAAATTAAGCATTCCCCGGACACTATATCTACTATAAACTACGGATCG
AAATGCATTTGAACAGCGGTTCCGGACACTATCTAGCGCTTGTCGGCTCAACGAGCCAATCCCG
TGATATTTTTTTTTTGCCGCACCATCCAATCACCGAGCCGGGTCAGGTCGCAAATCAAATGGGG
CTCTCGTTGGTTTGGGTTGGCCAACTGGCTGCCTCACCATCATCTGTCAGACTGTCACGTCCCG
GGTTGGCGCGGCGGCAGGTCGCAGCCACGTCGCCCGCCGAACCCCTCTTACAACCAGAGCCACA
CACACACCCCGCGCCGGTCTGCCAAGTGCCCAAACCCCCGTAGCGGACGCCAAAAAATCTCGTG
GCTTTCCCAGCGGCGAATAGGACGGCGACGGAGGGCAAAAAAAAAAGATAAAGCTAAAGCGAGG
TAAAAAAAGCGTTACAACCTTTTTAAGTTATTATTATTATTATTATTTCCCCGCCGCTAGGCTG
CCTGGTTGGTGTGGGTGTGGCACTGTGGGTTGTGTGGGTGGCACCTGCCCGGTTGGACCTGCAC
GACCCCGCGCTCTCAGCCGACGTGCGCGTCATCGCGTAGCGCACGACTGGGCCGGGCCGAGCCG
TCGCTGTCGCGGGGACGGCCGGCGCCGGGACGCAACGCACCGGCGGACCACTCTCGCCCACGGC
TGCTATCATTCATTGTGGTGTTGCTTACCGTGGATTGGAAAATTCTTCGGCCGACGTGGCTACA
TGCGGATTCCGAGAAAACGACCGCCCGAGCGATCGCAGCCTCGCGCCCTGGGCAAATGTTCTTT
CTTGTGGAACCCGTCGAAGGTTCTTCTACCACACTAGTACACTACTACCTGACTACCAGGTCGA
AGTGCTATACAACTAGTACGGCATACGATTCGTCCACCACACTAGCGCATGTTCTTGTGGCTCA
CAGGAACTTGTCAGGTTCTAACTAAACAGTGTCTGCAACGCTGTCCGTGCTTAACAATTTGTTA
GATTTTGGATGATGACGAACGATCATGTGCTTAACACGACAGCAAAAACCCGTCTGGGCAGGTG
CGGCGAGGATGAGGATGACGACATGGAGGAGCACGCGGTGCAGGTGCTGGTGCTGGCGCACGCG
TACCAGGTGCCGTGGCTGAAGCGGGCGTGCGAGGGCGCCATCGGCGCGCGCCTCACCGCGGACT
CGGTGGTGGACGTGCTGCAGCTGGCCGGCCTCTGCGACGCGCCGCGCCTGCACCTGCGCTGCGC
CAGGCTGCTGGCCAAGGAGTTCGCGGCCGTGGAGCGCACCGAGGCCTGGCGCTTCCTGCAGGAG
AACGACCCCTGGCAGGAGCTCCACGTCCTGCAGCGCCTGCACGAGGCCGACATGGTACGCACGC
TTTTTTTTCTTCTTCCATGGATTGCTACCGGACCGGACCGGACCGGACCACGGTCGGTCGATGT
AAATCTGACTGACACATGCTTGCACGCACCGCACCGAATGGAAGTGCGAGTGCGAAGAAATTAT
CATGCATGCATGCATGATGGCGCACGCCGGCGGGGTGGGGTGGGCACTGGGGAGCATGCTTTCT
TTACCGTCAAGCTTTTGTTCCAGGAAACGGTGGACATGGTCGGACCTCCGTGTCGCGTTCGTGC
GCGTACGGCGTACGAACGACATGACGCCAGTGTTCTTTTCTATGTGGTTGGTGCGTCTGTTTGG
GCCGGCCCGTCCACGCCGAATGGTCGGGCGAATGTTCGCATCTCTCCGACCTCTTACCTTTCCG
ACTCTGTTTTGATTTTACACGTTTTCATCTGGCTGGGGGTAGACAAATCGTGAGACCAAGTAAT
TTGTGCCTTGGTTTGACCAAAGTTAGCAGAAATCTTGGCGGCTACTCCATCAGTTCCATCTACA
AACATTCTTCGTAGGGCTTCAAAAGTCCAGCGGAGAACGTTTCACCAATAATAACGTTTGTTGG
AAACAACAGTTCCACGATGCACGTTTTTTTTTGTGTACCGACGACCGACCCAGCCTCTTAGCTA
AAGCACTGACCCGATGCGCGCTCGGTTCCTTCCTGGACATGCAGCGGCGGCGCAAGTGGCGGCG
GAAGCGCGCGGAGCAGCGCGTGTACATGGAGCTGAGCGAGGCCATGGACTGCCTGGACCACATC
TGCACGGAGGGATGCACGGAGGTCGGCCCGGCGGGGCGGGCGCCGGCGCCGGCGCCGTGCGCGC
GCTACGCCACGTGCCGGGGCCTGCAGCTGCTCATCCGCCACTTCTCCCAGTGCCACCGCAAGAG
CTGCGCGCGGTGCCAGCGCATGTGGCAGCTGCTCCGCCTCCACTCCGCGCTCTGCGACCGTACC
GACCGCTGCAACACCCCGCTCTGCATGTAAGCGCCGCGCCGCCGCTTGCTTCGTTGCAGCCATC
CATGGTCCATTGCGTGACCTCGCGCCTCACATTGTTGTGCTTGTGCCGTGCAGGAGGTTTAAGC
AGCAGGAGCAGGATAAGGCGGCTGCCAAGGGCGGCGATGATGGCGACAAATGGGGGCTTCTGGT
GAAGAAGGTGAAGACTGCCAGGGTCTTCTCTTCTCTGGCCAACAGGAAGCAGATGAGCACCACC
ACCACCCAGTGCTGAGGAGCAGCCTGGGATGCTTAGGGGTCAAAGTTAGATTAGTGAGAGAGAA
TGAAGCGAGCAGTTTTTTTTTGGGTCTGTTTTGAGATGTCAGATCTCTTTTGAGATATGTACAT
GTACATGCCTACTTTGTCACCCTACGTTGGTATAGCTCTTTTATCGAGGTGGGAGTTGGTCAGC
CGGAAGTCTATGTAAAAAGTGCGCATGTATCATTATATAATAATAGGGAAATATGAGAAGTATT
TTAGTCTTATGAT

SEQ ID NO: 14 shows a further exemplary BTB1 polypeptide (Zea mays GRMZM2G004161):

MCEAPRLGNRAGAGDPAAADVDVVTTGGRRRIPAHSSVLASASPVLGS
ILERRLRKDRESGKPGRSVVRIRGVTDDAAAAFVRLLYAGRCGEDEDD
DMEEHAVQVLVLAHAYQVPWLKRACEGAIGARLTADSVVDVLQLAGLC
DAPRLHLRCARLLAKEFAAVERTEAWRFLQENDPWQELHVLQRLHEAD
MRRRKWRRKRAEQRVYMELSEAMDCLDHICTEGCTEVGPAGRAPAPAP
CARYATCRGLQLLIRHFSQCHRKSCARCQRMWQLLRLHSALCDRTDRC
NTPLCMRFKQQEQDKAAAKGGDDGDKWGLLVKKVKTARVFSSLANRKQ
MSTTTTQC

SEQ ID NO:15 shows a further exemplary polynucleotide encoding a BTB1 polypeptide (Tritricum aestivum AK333270.1):

GAGCCTCCCTCCCCCGAAACCAACACCACACGCACGCCGCGACATTTCC
CAGATATTTCGCCCCTGCGCTTTTCTTCCTCCTCCCTTCCTTCCTTCTC
CGCCGCAATTGCTCCTAGTTATCCTTAGCACAGCATGCCGGAGGCACCT
GCACGGGCAGCCGGCGGCGGCGGCGGCGCCTCCGGCCCCGCCGACGTCG
ACGTCGTCACCTCCAGCGGCCGCCGCAAGATCGCCGCCCATTCCTCCGT
TCTTGCGTCGGCGTCGCCGGTGCTGGAGACCATCCTGGAGCGCCGGCTG
CAGAGAGTCAGGGAGAGCGGCAAGGGCGGCAGGGCCGTCGTCCGGATCC
GCGGCGTCACCGACGACGTCGCGGCGGCGTTCGTCCGCCTCCTCTACGC
CGGCAGCAGGCGTGGCGATGGTGAGGTGGAGGAGGAGGTGGAGAGGTAC
GCGGAGCAGCTGCTGGTGCTGGCGCACGCGTACCGGGTGCCGTGGCTGA
AGCGGTGGTGCCAGGAGGCCATCGGGTCGCGGCTCACCCCGGGCACCGT
GGTGGACGCGCTGCAGCTGGCCGACCTCTGCGACGCGCCGCAGCTGCAC
CTCCGCTGCATGCGCCTGCTCGCCAAGGAGTTCCGCGCCGTCGAGCGCA
CCGAGGCATGGCGCTTCCTCCGCGACAACGACCCCTGGCAGGAGCTCGA
CGTCCTCCGCCGCCTCCACGACGCCGACATGCGGCGGCGAAAGTGGCGT
CGGAAGCGCGCGGAGCAGAAGGTGTACGTGGAGCTGAGCGACGCCATGG
ACATCCTGCGGCACATCTGCACGGAGGGCTGCACGGAGGTCGGCCCTGT
GGGGCAGGCGCCGGCCAAGTCGCCGTGCCCGGCGTACGCGACGTGCCGG
GGCCTGCAGCTGCTCATCCGCCACTTCTCCCGGTGCAAGAGCCGCGCCA
CCTGCCCCCGCTGCCAGCGCATGTGGCAGCTGCTCCGCCTCCACGCCGC
GCTCTGCCGCGTCCCCGACGGCCACTGCAACACTCCTCTCTGCACGCAG
TTCAAGCTCAAGGAGCAGCAGAAGGAGGCGATGTCGGCTTCGGTGGCGG
CGAAGGCCGGCGACGGCAGGTGGGGGCTTCTTGTGAAGAAGGTGAAGGC
TGTCAGTGTCATGTCTTCCCTCGGCAAGAGAAGCTCGCCCTCTCAGTGC
TGCTGAGCCTGAGCGAATGGAATGTGTGTTACTGAGGATCCCCTCTCAG
AGCTGATCGAATGTTGTGCAATCACAACGAGAATCAGCCTGGGACAGGT
AGCTAGATGTAGTGTGCGAGTGTAGTGAAAGCGGGCTATTTTTTGGCCC
GTCTTTCCAGTGTATAATCTCTCTATTGAGCAATGTACATTAGTTATAT
CAATCATAGTGTTATTCTTTTGTGGGGCGAGAGAGGAAAAGGGTGGAGA
TTTGTTGTAAAATACTGCCTGCACTGTAATACTAGTAGCAAAGTATGGG
ATTTGTCCACATAGCCGA

SEQ ID NO:16 shows a further exemplary BTB1 polypeptide (Tritricum AK333270.1):

DVDVVTSSGRRKIAAHSSVLASASPVLETILERRLQRVRESGKGGRAVV
RIRGVTDDVAAAFVRLLYAGSRRGDGEVEEEVERYAEQLLVLAHAYRVP
WLKRWCQEAIGSRLTPGTVVDALQLADLCDAPQLHLRCMRLLAKEFRAV
ERTEAWRFLRDNDPWQELDVLRRLHDADMRRRKWRRKRAEQKVYVELSD
AMDILRHICTEGCTEVGPVGQAPAKSPCPAYATCRGLQLLIRHFSRCKS
RATCPRCQRMWQLLRLHAALCRVPDGHCNTPLCTQFKLKEQQEAMSASV
AAKAGDGRWGLLVKKVKAVSVMSSL

### DETAILED DESCRIPTION

### I. Overview of Several Embodiments

Development of genetic varieties with improved nitrogen use efficiency (NUE) is essential for sustainable agriculture. However, achieving this goal has proven difficult possibly due to the fact that NUE is a complex trait encompassing multiple physiological and developmental processes. This problem was addressed by taking a systems biology approach to identify candidate target genes.

First, a supervised machine learning algorithm was used to predict a NUE gene network in the model plant system, Arabidopsis thaliana. Second, network statistics were used to rank candidate genes, and identified BT2, a member of the Bric-a-Brac/Tramtrack/Broad (BTB) gene family, as the most central and connected gene in the NUE network. Third, BT2 were experimentally tested for a role in NUE by reverse genetic strategies.

Disclosed herein are the results that NUE decreases in plants overexpressing BT2, as compared to wild-type plants under limiting nitrate conditions. No difference was observed for bt2 mutant plants, though overexpression of BT2 was found to alter NUE. However, NUE increased (as compared to wild-type plants) under low nitrate conditions in double-mutant plants containing mutations in bt2, and also its closely-related homolog, bt1. This result indicates functional redundancy of BT1 and BT2 for NUE. Expression of the nitrate transporter genes NRT2.1 and NRT2.4 increased in the bt1/bt2 double mutant (as compared to wild-type plants), with a concomitant 65% increase in nitrate uptake under low nitrate conditions.

Our results demonstrate that a manipulatable genetic mechanism exists in planta to modulate NUE. BTB gene family members are at the center of a gene network acting as negative regulators of gene expression, nitrate uptake, and NUE. Given the results and guidance provided herein, those in the art are now in a position to utilize BTB genes in biotechnological strategies for the improvement of NUE in crops.

### II. Abbreviations

BNF biological nitrogen fixation
BTB Bric-a-Brac/Tramtrack/Broad
CRISPR clustered regularly interspaced short palindromic repeats
crRNA small CRISPR RNA
DLS Discriminative Local Subspaces (algorithm)
DNA deoxyribonucleic acid
DW dry weight
iRNA inhibitory ribonucleic acid
N nitrogen
NFB N-fixing bacterium
NUE nitrogen use efficiency
PAM protospacer adjacent motif
RNA ribonucleic acid
RNAi ribonucleic acid interference
tracrRNA trans-activating crRNA

### III. Terms

In the description and tables which follow, a number of terms are used. In order to provide a clear and consistent understanding of the specification and claims, including the scope to be given such terms, the following definitions are provided:

Backcrossing: Backcrossing methods may be used to introduce a nucleic acid sequence into plants. The backcrossing technique has been widely used for decades to introduce new traits into plants. Jensen, N., Ed. Plant Breeding Methodology, John Wiley & Sons, Inc., 1988. In a typical backcross protocol, the original variety of interest (recurrent parent) is crossed to a second variety (non-recurrent parent) that carries a gene of interest to be transferred. The resulting progeny from this cross are then crossed again to the recurrent parent, and the process is repeated until a plant is obtained wherein essentially all of the desired morphological and physiological characteristics of the recurrent plant are recovered in the converted plant, in addition to the transferred gene from the non-recurrent parent.

Isolated: An “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome). Nucleic acid molecules and proteins that have been “isolated” include nucleic acid molecules and proteins purified by standard purification methods, wherein there has been a chemical or functional change in the nucleic acid or protein. The term also embraces nucleic acids and proteins prepared by recombinant expression in a host cell, as well as chemically-synthesized nucleic acid molecules, proteins, and peptides.

Nucleic acid molecule: As used herein, the term “nucleic acid molecule” may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A nucleic acid molecule can include either or both naturally occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

Oligonucleotide: An oligonucleotide is a short nucleic acid molecule. Oligonucleotides may be formed by cleavage of longer nucleic acid segments, or by polymerizing individual nucleotide precursors. Automated synthesizers allow the synthesis of oligonucleotides up to several hundred base pairs in length. Because oligonucleotides may bind to a complementary nucleotide sequence, they may be used as probes for detecting DNA or RNA. Oligonucleotides composed of DNA (oligodeoxyribonucleotides) may be used in PCR, a technique for the amplification of small DNA sequences. In PCR, the oligonucleotide is typically referred to as a “primer,” which allows a DNA polymerase to extend the oligonucleotide and replicate the complementary strand.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, inter-nucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

As used herein with respect to DNA, the term “coding sequence” refers to a nucleotide sequence that is transcribed into RNA (e.g., mRNA and iRNA) when placed under the control of appropriate regulatory sequences. A “protein coding sequence” is a nucleotide sequence (DNA or RNA) that is ultimately translated into a polypeptide, via transcription and mRNA. With respect to RNA, the term “coding sequence” refers to a nucleotide sequence that is translated into a peptide, polypeptide, or protein. The boundaries of a coding sequence are determined by a translation start codon at the 5′-terminus and a translation stop codon at the 3′-terminus. Coding sequences include, but are not limited to: genomic DNA; cDNA; EST; and recombinant nucleotide sequences.

Genome: As used herein, the term “genome” refers to chromosomal DNA found within the nucleus of a cell, and also refers organelle DNA found within subcellular components of the cell. In some embodiments of the invention, a DNA molecule may be introduced into a plant cell such that the DNA molecule is integrated into the genome of the plant cell. In these and further embodiments, the DNA molecule may be either integrated into the nuclear DNA of the plant cell, or integrated into the DNA of the chloroplast or mitochondrion of the plant cell.

Endogenous: The term “endogenous,” as applied to nucleic acids (e.g., polynucleotides, DNA, RNA, and genes) herein, refers to one or more nucleic acid(s) that are normally (e.g., in a wild-type cell of the same type and species) present within their specific environment or context. For example, an endogenous gene is one that is normally found in the particular cell in question and in the same context (e.g., with regard to regulatory sequences). Endogenous nucleic acids can be distinguished from exogenous and/or heterologous, for example and without limitation, by detection in the latter of sequences that are consequent with recombination from bacterial plasmid; identification of atypical codon preferences; and amplification of atypical sequences in a PCR reaction from primers characterized in a wild-type cell.

Exogenous: The term “exogenous,” as applied to nucleic acids herein, refers to one or more nucleic acid(s) that are not normally present within their specific environment or context. For example, if a host cell is transformed with a nucleic acid that does not occur in the untransformed host cell in nature, then that nucleic acid is exogenous to the host cell. The term exogenous, as used herein, also refers to one or more polynucleotide(s) that are identical in sequence to a polynucleotide already present in a host cell, but that are located in a different cellular or genomic context than the polynucleotide with the same sequence already present in the host cell. For example, a polynucleotide that is integrated in the genome of the host cell in a different location than a polynucleotide with the same sequence is normally integrated in the genome of the host cell is exogenous to the host cell. Furthermore, a nucleic acid (e.g., a DNA molecule) that is present in a plasmid or vector in the host cell is exogenous to the host cell when a nucleic acid with the same sequence is only normally present in the genome of the host cell.

Heterologous: The term “heterologous,” as applied to nucleic acids (e.g., polynucleotides, DNA, RNA, and genes) herein, means of different origin. For example, if a host cell is transformed with a nucleic acid that does not occur in the untransformed host cell in nature, then that nucleic acid is heterologous (and exogenous) to the host cell. Furthermore, different elements (e.g., promoter, enhancer, coding sequence, terminator, etc.) of a transforming nucleic acid may be heterologous to one another and/or to the transformed host. The term heterologous, as used herein, may also be applied to one or more polynucleotide(s) that are identical in sequence to a polynucleotide already present in a host cell, but that are now linked to different additional sequences and/or are present at a different copy number, etc.

Sequence identity: The term “sequence identity” or “identity,” as used herein in the context of two polynucleotide or polypeptide sequences, may refer to the residues in the two sequences that are the same when aligned for maximum correspondence over a specified comparison window.

As used herein, the term “percentage of sequence identity” may refer to the value determined by comparing two optimally aligned sequences (e.g., nucleotide sequences, and amino acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method.

Specifically hybridizable/specifically complementary: As used herein, the terms “specifically hybridizable” and “specifically complementary” are terms that indicate a sufficient degree of complementarity such that stable and specific binding occurs between the nucleic acid molecule and a target polynucleotide. Stable and specific binding occurs when a nucleic acid molecule of interest (e.g., a primer or iRNA) binds to a target polynucleotide under stringent hybridization conditions, but does not bind to other polynucleotides under those conditions. Hybridization between two nucleic acid molecules involves the formation of an anti-parallel alignment between the nucleic acid sequences of the two nucleic acid molecules. The two molecules are then able to form hydrogen bonds with corresponding bases on the opposite strand to form a duplex molecule that, if it is sufficiently stable, is detectable using methods well known in the art. A nucleic acid molecule need not be 100% complementary to its target polynucleotide to be specifically hybridizable. However, the amount of sequence complementarity that must exist for hybridization to be specific is a function of the hybridization conditions used.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are known to those of ordinary skill in the art, and are discussed, for example, in Sambrook et al. (ed.) Molecular Cloning: A Laboratory Manual, 2^{nd} ed., vol. 1-3, Cold Spring Harbor Laboratory Press, Cold Spring Harbor, N.Y., 1989, chapters 9 and 11; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biology-Hybridization with Nucleic Acid Probes, Part I, Chapter 2, Elsevier, N Y, 1993; and Ausubel et al., Eds., Current Protocols in Molecular Biology, Chapter 2, Greene Publishing and Wiley-Interscience, NY, 1995.

As used herein, “stringent conditions” encompass conditions under which hybridization will only occur if there is less than 20% mismatch between the hybridization molecule and a homologous sequence within the target nucleic acid molecule. “Stringent conditions” include further particular levels of stringency. Thus, as used herein, “moderate stringency” conditions are those under which molecules with more than 20% sequence mismatch will not hybridize; conditions of “high stringency” are those under which sequences with more than 10% mismatch will not hybridize; and conditions of “very high stringency” are those under which sequences with more than 5% mismatch will not hybridize.

The following are representative, non-limiting hybridization conditions.

High Stringency condition (detects sequences that share at least 90% sequence identity): Hybridization in 5×SSC buffer at 65° C. for 16 hours; wash twice in 2×SSC buffer at room temperature for 15 minutes each; and wash twice in 0.5×SSC buffer at 65° C. for 20 minutes each.

Moderate Stringency condition (detects sequences that share at least 80% sequence identity): Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each.

Non-stringent control condition (sequences that share at least 50% sequence identity will hybridize): Hybridization in 6×SSC buffer at room temperature to 55° C. for 16-20 hours; wash at least twice in 2×-3×SSC buffer at room temperature to 55° C. for 20-30 minutes each.

As used herein, the term “substantially homologous” or “substantial homology,” with regard to polynucleotides, refers to polynucleotides that hybridize under stringent conditions to the reference polynucleotide. For example, polynucleotides that are substantially homologous to a reference DNA coding sequence are those polynucleotides that hybridize under stringent conditions (e.g., the Moderate Stringency conditions set forth, supra) to the reference DNA coding sequence. Substantially homologous sequences may have at least 80% sequence identity. For example, substantially homologous sequences may have from about 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target polynucleotides under conditions where specific binding is desired, for example, under stringent hybridization conditions.

As used herein, the term “ortholog” refers to a gene in two or more species that has evolved from a common ancestral nucleotide sequence, and may retain the same function in the two or more species.

As used herein, two polynucleotides are said to exhibit “complete complementarity” when every nucleotide of the sequence of a first polynucleotide read in the 5′ to 3′ direction is complementary to every nucleotide of the sequence of the other polynucleotide when read in the 3′ to 5′ direction. A polynucleotide that is complementary to a reference polynucleotide will exhibit a sequence (in the same direction of the hybridized duplex molecule) identical to the reverse complement sequence of the reference nucleotide sequence. These terms and descriptions are well defined in the art and are easily understood by those of ordinary skill in the art.

As used herein, the term “substantially identical” may refer to nucleotide sequences that are more than 85% identical. For example, a substantially identical nucleotide sequence may be at least 85.5%; at least 86%; at least 87%; at least 88%; at least 89%; at least 90%; at least 91%; at least 92%; at least 93%; at least 94%; at least 95%; at least 96%; at least 97%; at least 98%; at least 99%; or at least 99.5% identical to the reference sequence.

Expression: As used herein, “expression” of a coding sequence (for example, a gene or a transgene) refers to the process by which the coded information of a nucleic acid transcriptional unit (including, e.g., genomic DNA or cDNA) is converted into an operational, non-operational, or structural part of a cell (e.g., a protein and iRNA molecule). Gene expression can be influenced by external signals; for example, exposure of a cell, tissue, or organism to an agent that increases or decreases expression of a gene comprised therein. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, and/or through activation, inactivation, compartmentalization, or degradation of specific protein molecules after they have been made, or by combinations of any of the foregoing. Gene expression can be measured at the RNA level or the protein level by methods known in the art, including, without limitation, Northern blot, RT-PCR, Western blot, and in vitro, in situ, or in vivo protein activity assay(s).

Decrease expression: As used herein, the term “decrease expression” refers to a reduction in the level of expression, as well as to a quantitative decrease in the amount of an expression product produced from a template construct. In some embodiments, at least one heterologous antisense polynucleotide (e.g., iRNA) may be provided to a cell or organism that comprises an endogenous copy of a gene comprising the antisense target polynucleotide, so as to decrease the expression of the RNA or polypeptide encoded by the gene. In such embodiments, the decrease in expression may be determined by comparison of the amount of the polypeptide produced in the cell comprising the heterologous and endogenous polynucleotides, with the amount produced in the cell comprising only the endogenous gene. In some embodiments, a first polypeptide that decreases expression (e.g., BTB1 and/or BTB2) may be provided to a cell or organism, so as to decrease the expression of a second polypeptide (e.g., NRT2.1 and/or NRT2.4) encoded by a gene under the control of the first polypeptide. In such embodiments, the decrease in expression may be determined by comparison of the amount of the polypeptide produced from the gene in the presence of the first polypeptide, with the amount produced from the gene in the absence of the first polypeptide. In some embodiments, an antisense polynucleotide that decreases expression of a target gene (e.g., BT1 and/or BT2) may be provided to a cell or organism, so as to decrease the expression of the target gene. In such embodiments, the decrease in expression may be determined by comparison of the amount of the polypeptide produced from the target gene in the presence of the antisense polynucleotide, with the amount produced from the target gene in the absence of the antisense polynucleotide.

Inhibition: As used herein, the term “inhibition,” when used to describe an effect on a coding sequence (for example, a gene), refers to a measurable decrease in the cellular level of mRNA transcribed from the coding sequence and/or peptide, polypeptide, or protein product of the coding sequence. In some examples, expression of a coding sequence may be inhibited such that expression is approximately eliminated. “Specific inhibition” refers to the inhibition of a target coding sequence without consequently affecting expression of other coding sequences (e.g., genes) in the cell wherein the specific inhibition is being accomplished.

Operably linked: A first nucleotide sequence is operably linked with a second nucleic acid sequence when the first nucleic acid sequence is in a functional relationship with the second nucleic acid sequence. When recombinantly produced, operably linked nucleic acid sequences are generally contiguous, and, where necessary to join two protein-coding regions, in the same reading frame (e.g., in a polycistronic ORF). However, nucleic acids need not be contiguous to be operably linked.

The term, “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

Promoter: As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell.

Some embodiments herein include a “plant promoter.” A plant promoter is a promoter that is capable of initiating transcription in a plant cell.

Some embodiments herein include a “tissue-preferred promoter.” A tissue-preferred promoter is a promoter that is capable of initiating transcription under developmental control, and include, for example and without limitation: promoters that preferentially initiate transcription in leaves, pollen, tassels, roots, seeds, fibers, xylem vessels, tracheids, and sclerenchyma. Promoters that initiate transcription essentially only in certain tissues are referred to as “tissue-specific.” A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters.

Any inducible promoter may be used in some embodiments herein. See Ward et al. (1993) Plant Mol. Biol. 22:361-366. With an inducible promoter, the rate of transcription increases in response to an inducing agent. Exemplary inducible promoters include, but are not limited to: Promoters from the ACEI system that responds to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone (Schena et al. (1991) Proc. Natl. Acad. Sci. USA 88:10421-5).

In contrast to non-constitutive promoters, a “constitutive” promoter is a promoter that is active under most environmental conditions. Exemplary constitutive promoters include, but are not limited to: promoters from plant viruses, such as the 35S promoter from CaMV; promoters from rice actin genes; ubiquitin promoters; pEMU; MAS; maize H3 histone promoter; and the ALS promoter, XbaI/NcoI fragment 5′ to the Brassica napus ALS3 structural gene (or a nucleotide sequence similarity to said Xba1/NcoI fragment) (PCT International Patent Publication No. WO 96/30530).

Additionally, any tissue-specific or tissue-preferred promoter may be utilized in some embodiments of the invention. Plants transformed with a nucleic acid molecule comprising a coding sequence operably linked to a tissue-specific promoter may produce the product of the coding sequence exclusively, or preferentially, in a specific tissue. Exemplary tissue-specific or tissue-preferred promoters include, but are not limited to: a root-preferred promoter, such as that from the phaseolin gene; a leaf-specific and light-induced promoter such as that from cab or rubisco; an anther-specific promoter such as that from LAT52; a pollen-specific promoter such as that from Zm13; and a microspore-preferred promoter such as that from apg.

Conservative substitution: As used herein, the term “conservative substitution” refers to a substitution where an amino acid residue is substituted for another amino acid in the same class. A non-conservative amino acid substitution is one where the residues do not fall into the same class, for example, substitution of a basic amino acid for a neutral or non-polar amino acid. Classes of amino acids that may be defined for the purpose of performing a conservative substitution are known in the art.

In some embodiments, a conservative substitution includes the substitution of a first aliphatic amino acid for a second, different aliphatic amino acid. For example, if a first amino acid is one of Gly; Ala; Pro; Ile; Leu; Val; and Met, the first amino acid may be replaced by a second, different amino acid selected from Gly; Ala; Pro; Ile; Leu; Val; and Met. In particular examples, if a first amino acid is one of Gly; Ala; Pro; Ile; Leu; and Val, the first amino acid may be replaced by a second, different amino acid selected from Gly; Ala; Pro; Ile; Leu; and Val. In particular examples involving the substitution of hydrophobic aliphatic amino acids, if a first amino acid is one of Ala; Pro; Ile; Leu; and Val, the first amino acid may be replaced by a second, different amino acid selected from Ala; Pro; Ile; Leu; and Val.

In some embodiments, a conservative substitution includes the substitution of a first aromatic amino acid for a second, different aromatic amino acid. For example, if a first amino acid is one of His; Phe; Trp; and Tyr, the first amino acid may be replaced by a second, different amino acid selected from His; Phe; Trp; and Tyr. In particular examples involving the substitution of uncharged aromatic amino acids, if a first amino acid is one of Phe; Trp; and Tyr, the first amino acid may be replaced by a second, different amino acid selected from Phe; Trp; and Tyr.

In some embodiments, a conservative substitution includes the substitution of a first hydrophobic amino acid for a second, different hydrophobic amino acid. For example, if a first amino acid is one of Ala; Val; Ile; Leu; Met; Phe; Tyr; and Trp, the first amino acid may be replaced by a second, different amino acid selected from Ala; Val; Ile; Leu; Met; Phe; Tyr; and Trp. In particular examples involving the substitution of non-aromatic, hydrophobic amino acids, if a first amino acid is one of Ala; Val; Ile; Leu; and Met, the first amino acid may be replaced by a second, different amino acid selected from Ala; Val; Ile; Leu; and Met.

In some embodiments, a conservative substitution includes the substitution of a first polar amino acid for a second, different polar amino acid. For example, if a first amino acid is one of Ser; Thr; Asn; Gin; Cys; Gly; Pro; Arg; His; Lys; Asp; and Glu, the first amino acid may be replaced by a second, different amino acid selected from Ser; Thr; Asn; Gin; Cys; Gly; Pro; Arg; His; Lys; Asp; and Glu. In particular examples involving the substitution of uncharged, polar amino acids, if a first amino acid is one of Ser; Thr; Asn; Gin; Cys; Gly; and Pro, the first amino acid may be replaced by a second, different amino acid selected from Ser; Thr; Asn; Gin; Cys; Gly; and Pro. In particular examples involving the substitution of charged, polar amino acids, if a first amino acid is one of His; Arg; Lys; Asp; and Glu, the first amino acid may be replaced by a second, different amino acid selected from His; Arg; Lys; Asp; and Glu. In further examples involving the substitution of charged, polar amino acids, if a first amino acid is one of Arg; Lys; Asp; and Glu, the first amino acid may be replaced by a second, different amino acid selected from Arg; Lys; Asp; and Glu. In particular examples involving the substitution of positively charged (basic), polar amino acids, if a first amino acid is one of His; Arg; and Lys, the first amino acid may be replaced by a second, different amino acid selected from His; Arg; and Lys. In further examples involving the substitution of positively charged, polar amino acids, if a first amino acid is Arg or Lys, the first amino acid may be replaced by the other amino acid of Arg and Lys. In particular examples involving the substitution of negatively charged (acidic), polar amino acids, if a first amino acid is Asp or Glu, the first amino acid may be replaced by the other amino acid of Asp and Glu.

In some embodiments, a conservative substitution includes the substitution of a first electrically neutral amino acid for a second, different electrically neutral amino acid. For example, if a first amino acid is one of Gly; Ser; Thr; Cys; Asn; Gin; and Tyr, the first amino acid may be replaced by a second, different amino acid selected from Gly; Ser; Thr; Cys; Asn; Gin; and Tyr.

In some embodiments, a conservative substitution includes the substitution of a first non-polar amino acid for a second, different non-polar amino acid. For example, if a first amino acid is one of Ala; Val; Leu; Ile; Phe; Trp; Pro; and Met, the first amino acid may be replaced by a second, different amino acid selected from Ala; Val; Leu; Ile; Phe; Trp; Pro; and Met.

In many examples, the selection of a particular second amino acid to be used in a conservative substitution to replace a first amino acid may be made in order to maximize the number of the foregoing classes to which the first and second amino acids both belong. Thus, if the first amino acid is Ser (a polar, non-aromatic, and electrically neutral amino acid), the second amino acid may be another polar amino acid (i.e., Thr; Asn; Gin; Cys; Gly; Pro; Arg; His; Lys; Asp; or Glu); another non-aromatic amino acid (i.e., Thr; Asn; Gin; Cys; Gly; Pro; Arg; His; Lys; Asp; Glu; Ala; Ile; Leu; Val; or Met); or another electrically-neutral amino acid (i.e., Gly; Thr; Cys; Asn; Gin; or Tyr). However, it may be preferred that the second amino acid in this case be one of Thr; Asn; Gin; Cys; and Gly, because these amino acids share all the classifications according to polarity, non-aromaticity, and electrical neutrality. Additional criteria that may optionally be used to select a particular second amino acid to be used in a conservative substitution are known in the art. For example, when Thr; Asn; Gin; Cys; and Gly are available to be used in a conservative substitution for Ser, Cys may be eliminated from selection in order to avoid the formation of undesirable cross-linkages and/or disulfide bonds. Likewise, Gly may be eliminate from selection, because it lacks an alkyl side chain. In this case, Thr may be selected, e.g., in order to retain the functionality of a side chain hydroxyl group. The selection of the particular second amino acid to be used in a conservative substitution is ultimately, however, within the discretion of the skilled practitioner.

Nitrogen-limiting conditions: As used herein, the term “nitrogen-limiting conditions” refers to conditions wherein there is a limited amount of nitrogen sources (e.g., nitrate and ammonium) in the soil or culture medium. The amount that is “limiting” is in some examples a range of nitrogen concentration from 0.0 to 0.2 mM; e.g., from 0 to 0.1 mM, from 0 to 0.03 mM, and from 0 to 0.05 mM.

Trait or phenotype: The terms “trait” and “phenotype” are used interchangeably herein. For the purposes of the present disclosure, traits of particular interest include agronomically important traits, as may be expressed, for example, in a crop plant.

Transformation: As used herein, the term “transformation” refers to the transfer of one or more nucleic acid molecule(s) into a cell. A cell is “transformed” by a nucleic acid molecule introduced into the cell when the nucleic acid molecule becomes stably replicated by the cell, either by incorporation of the nucleic acid molecule into the cellular genome, or by episomal replication. As used herein, the term “transformation” encompasses all techniques by which a nucleic acid molecule can be introduced into such a cell. Examples include, but are not limited to: transfection with viral vectors; transformation with plasmid vectors; electroporation (Fromm et al. (1986) Nature 319:791-3); lipofection (Felgner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; and microprojectile bombardment (Klein et al. (1987) Nature 327:70).

Transgene: A transgene is an exogenous nucleic acid sequence. In some examples, a transgene may be a sequence that encodes one or both strand(s) of a dsRNA molecule that comprises a nucleotide sequence that is complementary to a target nucleic acid. In some examples, a transgene may be an antisense nucleic acid sequence, the expression of which inhibits expression of a target nucleic acid. In still other examples, a transgene may be a gene sequence (e.g., a herbicide-resistance gene), a gene encoding an industrially or pharmaceutically useful compound, or a gene encoding a desirable agricultural trait. In these and other examples, a transgene may contain regulatory sequences operably linked to the coding sequence of the transgene (e.g., a promoter).

Vector: A vector refers to a nucleic acid molecule as introduced into a cell, for example, to produce a transformed cell. A vector may include nucleic acid sequences that permit it to replicate in the host cell, such as an origin of replication. Examples of vectors include, but are not limited to: a plasmid; cosmid; bacteriophage; and a virus that carries exogenous DNA into a cell. A vector may also include one or more genes, antisense molecules, and/or selectable marker genes and other genetic elements known in the art. A vector may transduce, transform, or infect a cell, thereby causing the cell to express the nucleic acid molecules and/or proteins encoded by the vector. A vector optionally includes materials to aid in achieving entry of the nucleic acid molecule into the cell (e.g., a liposome, protein coating, etc.).

Unless specifically indicated or implied, the terms “a,” “an,” and “the” signify “at least one,” as used herein.

Unless otherwise specifically explained, all technical and scientific terms used herein have the same meaning as commonly understood by those of ordinary skill in the art to which this disclosure belongs. Definitions of common terms in molecular biology can be found in, for example, Lewin B., Genes V, Oxford University Press, 1994 (ISBN 0-19-854287-9); Kendrew et al. (eds.), Blackwell Science Ltd., 1994 (ISBN 0-632-02182-9); and Meyers R. A. (ed.), Molecular Biology and Biotechnology: A Comprehensive Desk Reference, VCH Publishers, Inc., 1995 (ISBN 1-56081-569-8). All percentages are by weight and all solvent mixture proportions are by volume unless otherwise noted. All temperatures are in degrees Celsius.

### IV. BTB Genes

This disclosure provides compositions and methods that exploit the surprising and unexpected finding that the BTB gene family (e.g., BT1 and BT2) are a manipulable genetic mechanism that modulates NUE in planta. As disclosed herein, BTB1 and BTB2 influence the physiological response of plants to limiting nitrate conditions, for example, by modulating the expression of cellular nitrate transporters. Thus, for example, BTB1 and/or BTB2 may be used to regulate the utilization of nitrogen by a plant. The properties of BTB1 and BTB2 described herein may be used, for example, to provide transgenic plants with an altered NUE phenotype. For example, expression of BTB1 and BTB2 may be decreased in a plant to increase the efficiency of the plant's utilization of environmental nitrogen (i.e., to increase NUE). Expression of either of BTB1 and BTB2 may be decreased in a plant, for example and without limitation, as part of a strategy to produce a double-mutant plant having decreased expression of both genes.

BTBs are scaffold proteins that are characterized by their protein-protein interaction domains. The genome of the model plant system, Arabidopsis, contains several genes that encode BTBs. Arabidopsis BTB1 and BTB2 are involved in several processes, including auxin response and telomerase activity in leaves (Ren et al. (2007), supra), gametophyte development (Robert et al. (2009) Plant J. 58(1):109-21), and light signals, nutrient status, hormones, and stress signaling (Mandadi et al. (2009), supra), indicating they act as integrators of multiple cellular pathways. Two-hybrid analysis showed that BTBs are able to interact with CULLIN3, and thus might form part of E3 Ubiquitin ligase complexes. Du & Poovaiah (2004) Plant Mol. Biol. 54(4):549-69. BTBs are also able to interact with bromodomain-containing proteins, BET9 and BET10. Du & Poovaiah (2004), supra. Bromodomain-containing proteins are able to interact and recognize acetylated lysines in histones, regulating transcription of target genes.

BTB proteins are predicted to respond to Ca²⁺-signals due to the presence of its calmodulin binding domain at the C-terminus. Du & Poovaiah (2004), supra. We recently showed that Ca²⁺ acts as a second messenger in the plant nitrate signaling pathway. Riveras et al. (2015) Plant Physiol., August 24. pii: pp. 00961.2015 (Epub ahead of print). Calcium-dependent protein kinases are key elements of nitrate signaling, including CIPK8, a regulator of primary nitrate responsive genes (Hu et al. (2009) Plant J. 57:264-78), and CIPK23, a kinase that phosphorylates the NPF6.3/NRT1.1 nitrate transceptor (Ho et al. (2009) Cell 138(6):1184-94. Accordingly, calcium signals triggered by nitrate availability likely control BTB-mediated changes in gene expression.

Some embodiments include a BTB polynucleotide and/or the BTB polypeptide encoded thereby. Particular embodiments include a BT1 (BTB1) polynucleotide and/or polypeptide, and/or a BT2 (BTB2) polynucleotide and/or polypeptide.

Particular embodiments include a BTB1 polypeptide. BTB1 polypeptides according to particular embodiments comprise an amino acid sequence showing increasing percentage identities when aligned with SEQ ID NO:2 (Arabidopsis thaliana BTB1). Specific polypeptides within these and other embodiments may comprise amino acid sequences having, for example, at least about 50%, about 55%, about 60%, about 65%, about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identity with SEQ ID NO:2. For example, some embodiments include a BTB1 ortholog, such as may be cloned from a crop plant by examining the predicted translation products of nucleic acids therein, or in the sequenced genome thereof. Methods of identifying such orthologs from the reference polypeptide of SEQ ID NO:2, for example, from any of the many published plant proteomes and genomes, are well-known in the art, and it is unnecessary to list the same here. Accordingly, BTB1 polypeptides are identified, for example, by locating polypeptide sequences having a threshold sequence identity with SEQ ID NO:2 in one of the many known sequence databases.

Particular embodiments include, or further include, a BTB2 polypeptide. BTB2 polypeptides according to particular embodiments comprise an amino acid sequence showing increasing percentage identities when aligned with SEQ ID NO:4 (Arabidopsis thaliana BTB2). Specific polypeptides within these and other embodiments may comprise amino acid sequences having, for example, at least about 50%, about 55%, about 60%, about 65%, about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identity with SEQ ID NO:4. For example, some embodiments include a BTB2 ortholog. Accordingly, BTB2 polypeptides are identified, for example, by locating polypeptide sequences having a threshold sequence identity with SEQ ID NO:4 in one of the many known sequence databases.

Useful sequence databases may be searched by any of many methods known to those of skill in the art (e.g., utilizing NCBI's BLAST® tool). Other databases are available for many plants and other organisms through a variety of public and private commercial sources. As will be appreciated by those of skill in the art, BTB1 and BTB2 are homologous proteins, and thus, a particular polypeptide identified as comprising an amino acid sequence sharing sequence identity with SEQ ID NO:2 or SEQ ID NO:4 may also share sequence identity with the other of SEQ ID NOs:2 and 4.

Some embodiments include a mutant bt polynucleotide and/or the mutant btb polypeptide encoded thereby. Mutant bt polynucleotides or polypeptides that result in decreased BTB function in a host plant cell wherein they are contained may be utilized to generate genetically-modified plant materials and plants that exhibit increased NUE. Particular, non-limiting examples of such mutant bt polynucleotides and polypeptides include the bt1 polynucleotide of SEQ ID NO:7 and/or the bt2 polynucleotide of SEQ ID NO:9, and the polypeptide products thereof.

Mutant btb polypeptides may be easily derived in a straightforward manner from, for example, the reference polypeptides of SEQ ID NO:2 (BTB1) and SEQ ID NO:4 (BTB2). For example, those in the art understand that significantly truncated polypeptides derived from these reference polypeptides will not recapitulate the function of the reference polypeptide in vitro or in vivo. Furthermore, polypeptides comprising a significant deletion also will not recapitulate the function of the reference polypeptide. Mutant btb polypeptides also include, but are not limited to, mutants comprising non-conservative substitutions of conserved amino acid residues within the amino acid sequence of the BTB1 or BTB2 polypeptides herein. As used herein, a “non-conservative” amino acid substitution is one where the amino acid residues that are substituted do not fall into the same class, for example, substitution of a basic amino acid for a neutral or non-polar amino acid. The amino acid homology of peptides can be readily determined by contrasting the amino acid sequences thereof as is known in the art. Similarly, the amphiphilic homology of peptides can be determined by contrasting the hydrophilicity and hydrophobicity of the amino acid sequences. Classes of amino acids that may be defined for the purpose of performing a non-conservative substitution are known in the art.

Hydrophilic amino acids generally include and generally have the respective relative degree of hydrophobicity (at pH 7.0; kcal/mol) as follows: aspartic acid (D), −7.4; glutamic acid (E)-9.9; asparagine (N), −0.2; glutamine (Q), −0.3; lysine (K), −4.2; arginine (R), −11.2; serine (S), −0.3; and cysteine (C), −2.8. Hydrophobic amino acids generally include and generally have the respective relative degree of hydrophobicity as follows: histidine (H), 0.5; threonine (T), 0.4; tyrosine (Y), 2.3; tryptophan (W), 3.4; phenylalanine (F), 2.5; leucine (L), 1.8; isoleucine (I), 2.5; methionine (M), 1.3; valine (V), 1.5; and alanine (A), 0.5. Glycine has a relative degree of hydrophobicity of 0 and may be considered to be hydrophilic or hydrophobic.

In some embodiments, a non-conservative substitution includes the substitution of a non-aliphatic amino acid for a conserved aliphatic amino acid. For example, if the conserved aliphatic amino acid is one of Gly; Ala; Pro; Ile; Leu; Val; and Met, the amino acid may be replaced by a second, different amino acid that is not Gly, Ala, Pro, Ile, Leu, Val, or Met.

In some embodiments, a non-conservative substitution includes the substitution of a non-aromatic amino acid for a conserved aromatic amino acid. For example, if the conserved aromatic amino acid is one of His; Phe; Trp; and Tyr, the amino acid may be replaced by a second, different amino acid that is not His, Phe, Trp, or Tyr.

In some embodiments, a non-conservative substitution includes the substitution of a non-hydrophobic amino acid for a conserved hydrophobic amino acid. For example, if the conserved hydrophobic amino acid is one of Ala; Val; Ile; Leu; Met; Phe; Tyr; and Trp, the amino acid may be replaced by a second, different amino acid that is not Ala, Val, lie, Leu, Met, Phe, Tyr, and Trp.

In some embodiments, a non-conservative substitution includes the substitution of a non-polar amino acid for a conserved polar amino acid. For example, if the conserved polar amino acid is one of Ser; Thr; Asn; Gln; Cys; Gly; Pro; Arg; His; Lys; Asp; and Glu, the amino acid may be replaced by a second, different amino acid that is not Ser, Thr, Asn, Gin, Cys, Gly, Pro, Arg, His, Lys, Asp, or Glu. In particular examples involving the mutation of uncharged, polar amino acids, if the conserved uncharged, polar amino acid is one of Ser; Thr; Asn; Gln; Cys; Gly; and Pro, the amino acid may be replaced by a second, different amino acid that is not Ser, Thr, Asn, Gin, Cys, Gly, or Pro. In particular examples involving the mutation of charged, polar amino acids, if the conserved charged, polar amino acid is one of His; Arg; Lys; Asp; and Glu, the amino acid may be replaced by a second, different amino acid that is not His, Arg, Lys, Asp, or Glu. In particular examples involving the mutation of positively-charged (basic), polar amino acids, if the conserved positively-charged, polar amino acid is one of His, Arg, and Lys, the amino acid may be replaced by a second, different amino acid that is not His, Arg, or Lys. In particular examples involving the mutation of negatively-charged (acidic), polar amino acids, if the conserved negatively-charged, polar amino acid is Asp or Glu, the amino acid may be replaced by an amino acid that is not Asp or Glu.

In some embodiments, a non-conservative substitution includes the substitution of an electrically charged amino acid for a conserved electrically neutral amino acid. For example, if the conserved electrically neutral amino acid is one of Gly; Ser; Thr; Cys; Asn; Gln; and Tyr, the amino acid may be replaced by a second, different amino acid that is not Gly, Ser, Thr, Cys, Asn, Gln, and Tyr.

In many examples, the selection of a particular second amino acid to be used in a non-conservative substitution to replace a first amino acid may be made in order to minimize the number of the foregoing classes to which the first and second amino acids both belong. Thus, if the first amino acid is Ser (a polar, non-aromatic, and electrically neutral amino acid), the second amino acid may be a non-polar amino acid (i.e., Ala; Val; Leu; lie; Phe; Trp; Pro; or Met); an aromatic amino acid (i.e., His; Phe; Trp; or Tyr); or an electrically-charged amino acid (i.e., His; Arg; Lys; Asp; or Glu). However, it may be preferred that the second amino acid in this case be Phe or Trp, because these amino acids share two of the three classifications according to polarity, non-aromaticity, and electrical neutrality. Additional criteria that may optionally be used to select a particular second amino acid to be used in a non-conservative substitution are known in the art. For example, when Thr; Asn; Gin; Cys; and Gly are available to be used in a non-conservative substitution for Ser, Cys may be selected in order to initiate the formation of cross-linkages and/or disulfide bonds. Likewise, Gly may be selected, because it lacks an alkyl side chain. In this case, Thr may be eliminated from selection, e.g., because it retains the functionality of a side chain hydroxyl group. The selection of the particular second amino acid to be used in a non-conservative substitution is ultimately, however, within the discretion of the skilled practitioner.

Some embodiments include a nucleic acid comprising a polynucleotide encoding a BTB1 polypeptide (a “BT1 polynucleotide”), a BTB2 polypeptide (a “BT2 polynucleotide”), a mutant btb1 polypeptide (a “bt1 polynucleotide”), and/or a mutant btb2 polypeptide (a “bt2 polynucleotide”), such as are described above. For example, nucleic acid sequences in some embodiments show increasing percentage identities when aligned with SEQ ID NO:2 (A. thaliana BTB1) and/or SEQ ID NO:4 (A. thaliana BTB2). Specific nucleic acid sequences within these and other embodiments may comprise sequences having, for example and without limitation, at least 50%, at least 55%, at least 60%, at least 65%, at least 70%, at least 75%, at least 80%, 81%, 82%, 83%, 84%, 85%, 86%, 87%, 88%, 89%, 90%, 91%, 92%, 93%, 94%, 95% 96%, 97%, 98%, 99%, or 100% identity with SEQ ID NO:1, SEQ ID NO:3, SEQ ID NO:7, and/or SEQ ID NO:9. In particular examples, the foregoing polynucleotides encode at least one of SEQ ID NO:2, SEQ ID NO:4, SEQ ID NO:8, and SEQ ID NO:10.

A large number of nucleic acids comprising a polynucleotide encoding a BTB1, BTB2, btb1, or btb2 polypeptide can be readily identified by those of skill in the art. For example, nucleic acid molecules may be modified without substantially changing the amino acid sequence of the encoded polypeptide, for example, by introducing permissible nucleotide substitutions according to codon degeneracy. Thus, it will be understood that any BTB1, BTB2, btb1, or btb2 polypeptide with a given amino acid sequence may be immediately reverse-engineered to any of many redundant nucleotide sequences. By way of further example, genes encoding a BTB1, BTB2, btb1, or btb2 polypeptide may be selected from any of the many available plant genomic libraries, cDNA libraries, EST libraries, and the like (e.g., by homology to SEQ ID NO:1 and/or SEQ ID NO:3), or by sequence similarity of an encoded polypeptide with SEQ ID NO:2 and/or SEQ ID NO:4, or such genes may be cloned from an organism according to reliable and well-known techniques in molecular biology.

Any and all BTB1, BTB2, btb1, and btb2 polypeptides, and nucleic acid molecules encoding the same, may be utilized in certain embodiments of the invention.

In some embodiments herein, a nucleic acid comprising a polynucleotide encoding a BTB1, BTB2, btb1, or btb2 polypeptide comprises a gene regulatory element (e.g., a promoter). Promoters may be selected on the basis of the cell type into which the vector construct will be inserted. Promoters which function in bacteria, yeast, and plants are well-known in the art. The promoters may also be selected on the basis of their regulatory features. Examples of such features include enhancement of transcriptional activity, inducibility, tissue-specificity, and developmental stage-specificity. In plants, promoters that are inducible, of viral or synthetic origin, constitutively active, temporally regulated, and spatially regulated have been described. See, e.g., Poszkowski et al. (1989) EMBO J. 3:2719; Odell et al. (1985) Nature 313:810; and Chau et al. (1989) Science 244:174-81).

To obtain higher expression of a heterologous gene(s), it may be preferred to reengineer the gene(s) so that it is more efficiently expressed in the expression host cell (e.g., a plant cell, for example, canola, rice, tobacco, maize, cotton, and soybean). Therefore, an optional additional step in the design of a gene encoding a BTB1, BTB2, btb1, or btb2 polypeptide for plant expression (i.e., in addition to the provision of one or more gene regulatory elements) is reengineering of a heterologous gene protein coding region for optimal expression. Particular examples include a redesigned Arabidopsis gene that has been optimized to increase the expression level (i.e. produce more protein) in a transgenic plant cell from a second plant species than in a plant cell from the second plant species transformed with the original (i.e., unmodified) Arabidopsis gene sequence.

Due to the plasticity afforded by the redundancy/degeneracy of the genetic code (i.e., some amino acids are specified by more than one codon), evolution of the genomes in different organisms or classes of organisms has resulted in differential usage of synonymous codons. This “codon bias” is reflected in the mean base composition of protein coding regions. For example, organisms having genomes with relatively low G+C contents utilize more codons having A or T in the third position of synonymous codons, whereas those having higher G+C contents utilize more codons having G or C in the third position. Further, it is thought that the presence of “minor” codons within an mRNA may reduce the absolute translation rate of that mRNA, especially when the relative abundance of the charged tRNA corresponding to the minor codon is low. An extension of this reasoning is that the diminution of translation rate by individual minor codons would be at least additive for multiple minor codons. Therefore, mRNAs having high relative contents of minor codons in a particular expression host would have correspondingly low translation rates. This rate may be reflected by correspondingly low levels of the encoded protein.

In engineering optimized genes encoding a BTB1, BTB2, btb1, or btb2 polypeptide for expression in a plant cell, it is helpful if the codon bias of the prospective host plant(s) has been determined. Multiple publicly-available DNA sequence databases exist wherein one may find information about the codon distribution of plant genomes or the protein coding regions of various plant genes.

The codon bias is the statistical distribution of codons that the expression host uses for coding the amino acids of its proteins. The codon bias can be calculated as the frequency at which a single codon is used relative to the codons for all amino acids. Alternatively, the codon bias may be calculated as the frequency at which a single codon is used to encode a particular amino acid, relative to all the other codons for that amino acid (synonymous codons).

In designing optimized coding regions for plant expression of BTB1, BTB2, btb1, or btb2 polypeptides, the primary (“first choice”) codons preferred by the plant should be determined, as well as the second, third, fourth etc. choices of preferred codons when multiple choices exist. A new DNA sequence can then be designed which encodes the amino sequence of the BTB1, BTB2, btb1, or btb2 polypeptide, wherein the new DNA sequence differs from the native DNA sequence (encoding the polypeptide) by the substitution of expression host-preferred (first preferred, second preferred, third preferred, or fourth preferred, etc.) codons to specify the amino acid at each position within the amino acid sequence. The new sequence is then analyzed for restriction enzyme sites that might have been created by the modifications. The identified putative restriction sites are further modified by replacing these codons with a next-preferred codon to remove the restriction site. Other sites in the sequence which may affect transcription or translation of heterologous sequence are exon:intron junctions (5′ or 3′), poly-A addition signals, and/or RNA polymerase termination signals. The sequence may be further analyzed and modified to reduce the frequency of TA or CG doublets. In addition to these doublets, sequence blocks that have more than about six G or C nucleotides that are the same may also adversely affect transcription or translation of the sequence. Therefore, these blocks are advantageously modified by replacing the codons of first or second choice, etc. with the next-preferred codon of choice.

A method such as that described above enables one skilled in the art to modify gene(s) that are foreign to a particular plant so that the genes are optimally expressed in plants. The method is further illustrated in PCT International Patent Publication No. WO 97/13402 A1. Thus, optimized synthetic genes that are functionally equivalent to BT1, BT2, bt1, and bt2 polynucleotides of some embodiments may be used to transform hosts, including plants and plant cells. Furthermore, BT1, BT2, bt1, and bt2 polynucleotides may also be generated, in silico, from an initial amino acid sequence. Additional guidance regarding the production of synthetic genes can be found in, for example, U.S. Pat. No. 5,380,831.

Once a BT1, BT2, bt1, or bt2 polynucleotide sequence has been designed on paper or in silico, actual nucleic acid molecules comprising the polynucleotide sequence can be synthesized in the laboratory to correspond in sequence precisely to the designed sequence. Such synthetic DNA molecules may be cloned and otherwise manipulated exactly as if they were derived from natural or native sources.

Some embodiments herein include iRNA molecules useful for decreasing the expression of a BT gene in a plant cell, thereby decreasing the BTB activity in the cell. Sucleic acid molecules include target sequences (e.g., native BT1 and BT2 genes, and operably linked non-coding sequences), dsRNAs, siRNAs, hpRNAs, and miRNAs. For example, dsRNA, siRNA, miRNA and/or hpRNA molecules in some embodiments may be specifically complementary to all or part of one or more native BT1 and BT2 polynucleotides in a plant. When such iRNAs are introduced into a plant cell comprising at least one native polynucleotide(s) to which the iRNAs are specifically complementary, RNAi is initiated in the cell, and consequently expression of the native polynucleotides is reduced or eliminated in the cell. In some examples, reduction or elimination of the expression of a BT1 or BT2 target gene in a plant by a nucleic acid molecule comprising a polynucleotide specifically complementary thereto increases NUE in the plant.

Accordingly, provided are polynucleotides, the expression of which results in a RNA molecule comprising a nucleotide sequence that is specifically complementary to all or part of a native RNA molecule that is encoded by a BT1 or BT2 coding sequence in a plant. In some embodiments, target BT1 or BT2 sequences include transcribed non-coding RNA sequences, such as 5′UTRs; 3′UTRs; spliced leader sequences; intron sequences; outron sequences (e.g., 5′UTR RNA subsequently modified in trans splicing); donatron sequences (e.g., non-coding RNA required to provide donor sequences for trans splicing); and other non-coding transcribed RNA of target BT1 and BT2 genes. Such sequences may be derived from both mono-cistronic and poly-cistronic genes.

Thus, also described herein in connection with some embodiments are iRNA molecules (e.g., dsRNAs, siRNAs, miRNAs and hpRNAs) that comprise at least one nucleotide sequence that is specifically complementary to all or part of a target BT1 or BT2 sequence in a plant. In some embodiments, an iRNA molecule may comprise nucleotide sequence(s) that are complementary to all or part of a plurality of target sequences; for example, both of BT1 and BT2 target sequences. In particular embodiments, an iRNA molecule may be produced in vitro, or in vivo by a genetically-modified organism, such as a plant or bacterium. Also disclosed are cDNA sequences that may be used for the production of dsRNA molecules, siRNA molecules, miRNA and/or hpRNA molecules that are specifically complementary to all or part of a target BT1 or BT2 sequence. Further described are recombinant DNA constructs for use in achieving stable transformation of particular host targets. Transformed host targets may express effective levels of dsRNA, siRNA, miRNA and/or hpRNA molecules from the recombinant DNA constructs. Therefore, also described is a plant transformation vector comprising at least one polynucleotide operably linked to a heterologous promoter functional in a plant cell, wherein expression of the polynucleotide(s) results in an iRNA molecule comprising a nucleotide sequence that is specifically complementary to all or part of a target BT1 or BT2 sequence in a host plant.

### V. Alteration of Plant Growth in N-Limiting Conditions by a BTB Knockout or Mutant

Some embodiments exploit the discovery that BTB1 and BTB2 function in plants to increase the efficiency with which the plant utilizes environmental nitrogen, thereby maintaining and/or increasing growth of the plant in N-limiting growth conditions. For example, BTB1 and BTB2 polypeptides may be replaced in a plant cell by mutant btb1 and/or btb2 polypeptides, for example, through homologous recombination between genomic DNA and an exogenous nucleic acid molecule (e.g., a vector), or by introgressing the mutant bt1 and/or bt2 alleles via plant breeding. By way of further example, the expression of BTB1 and BTB2 polypeptides may be reduced or eliminated through RNAi.

Disclosed herein is the result that BTB polypeptides are part of a central metabolic switch that manages offer and demand in plants. In the model plant system, Arabidopsis, this fact is evident from the early vegetative stage of plant development, where BTB polypeptides work as an early developmental brake that limits plant growth, adapting development, and growth to N availability. This “brake mechanism” may be, at least in part, mediated by controlling nitrate uptake by any of NRT2 transporters and other BTB targets. In some plants, as modeled in Arabidopsis, N uptake primarily occurs during the vegetative stage (as compared to the reproductive and subsequent developmental phases in the in plants). Malagoli et al. (2004) Plant Physiol. 134(1):388-400; Beuve et al. (2004) Plant Cell Environ. 27:1035-46; Masclaux-Daubresse et al. (2010), supra. This is in accordance with a role of BTB polypeptides during Arabidopsis early development, when nitrate uptake has a more prominent role in determining plant N status.

In particular embodiments, expression of a BTB1 and/or BTB2 polypeptide may be decreased or eliminated in a cell or organism, for example and without limitation, by disrupting, mutating, or inactivating a BT1 and/or BT2 polynucleotide; introducing an antisense nucleic acid into the cell or organism that targets a BT1 and/or BT2 polynucleotide; by physically removing the BTB1 and/or BTB2 polypeptide from the cellular machinery of the cell or organism by binding the BTB1 and/or BTB2 polypeptide with antibodies or other specific binding proteins; and/or by providing positive or negative signals sufficient to reduce or eliminate expression of the BTB1 and/or BTB2 polypeptide through an interaction of the signal(s) with regulatory elements operably linked to a BT and/or BT2 polynucleotide in the cell or organism. In specific embodiments, a BTB1 and/or BTB2 polypeptide may be decreased or eliminated in a cell or organism by introducing a mutant bt1 and/or bt2 polynucleotide into the cell or organism; and by introducing a polynucleotide into the cell or organism that decreases expression of the BTB1 and/or BTB2 polypeptide through RNA interference.

It is disclosed herein that BTB1 and BTB2 have functional redundancy with regard to the regulation of NUE. Therefore, in some embodiments, both BTB1 and BTB2 polypeptides may be reduced or eliminated in a plant cell or organism, so as to increase NUE in the cell or organism. In further embodiments, a BTB1 or BTB2 polypeptide may be singly reduced or eliminated in a plant cell or organism; for example and without limitation, as a first step in producing a cell or organism having increased NUE.

In particular embodiments, a mutant btb1 and/or btb2 polypeptide is expressed from a polynucleotide that is operably linked to regulatory elements that direct the expression of the polypeptide(s) in conditions other than those where nitrogen is growth limiting, thereby increasing the efficiency with which the plant utilizes environmental nitrogen under those other conditions.

In some embodiments herein, a plant material (e.g., plant cell, plant part, plant tissue, plant organ, and plant cell or tissue culture) and/or plant may be genetically modified to comprise at least one BT1 and/or BT2 polynucleotide knockout event, mutant bt1 and/or bt2 gene, and/or polynucleotide encoding an iRNA targeting a BT1 and/or BT2 gene, by any of several methods of introducing a heterologous molecule known in the art, thereby producing a non-natural transgenic plant material and/or plant. In particular embodiments herein, a heterologous molecule is introduced into a plant material or plant by a method selected from, for example and without limitation: transformation and selective breeding (e.g., backcross breeding).

In some embodiments, a mutant bt1 and/or bt2 polynucleotide and/or polynucleotide encoding an iRNA targeting a BT1 and/or BT2 gene is introduced such that it is operably linked to a constitutive promoter, so as to direct the expression of the polynucleotide under all conditions (i.e., whether nitrogen is limited or not limited). In particular embodiments, the polynucleotide is introduced such that it is operably linked to a non-constitutive promoter, so as to direct the expression of the gene products in a tissue-preferred (e.g., in root tissue) or tissue-specific manner. In particular embodiments, the polynucleotide is introduced such that it is operably linked to an inducible promoter, so as to direct the expression of the gene products in a controlled manner (e.g., when nitrogen is limited).

In some embodiments, a CRISPR-based genetic engineering system is utilized to introduce a mutation into a BTB gene, for example, to reduce or eliminate expression of the gene. A CRISPR-based genetic engineering system comprises a guide RNA and an endonuclease, for example, a CRISPR-associated (Cas) nuclease (e.g., Cas9). The guide RNA is a single chimeric transcript that combines an endogenous bacterial crRNA and tracrRNA. The guide RNA combines the targeting specificity of the crRNA with the scaffolding properties of the tracrRNA into a single transcript. When the gRNA and the Cas nuclease are expressed in the cell, the genomic target sequence is modified or permanently disrupted.

The gRNA/Cas complex is recruited to the target sequence by base-pairing between the guide RNA and the complement to the target sequence in the genomic DNA. For successful binding of the Cas nuclease, the genomic target sequence contains a PAM sequence immediately following the target sequence. The binding of the gRNA/Cas complex localizes the Cas nuclease to the target sequence, such that both strands of DNA are cleaved 3-4 nucleotides upstream of the PAM sequence, causing a double strand break. The double strand break is then repaired through one of two general repair pathways: the non-homologous end joining DNA repair pathway; or the homology directed repair pathway. The non-homologous end joining DNA repair pathway is used to create inserts/deletions (InDels) at the DSB site (for example, that lead to a frameshift and/or premature stop codon), effectively disrupting the open reading frame (ORF) of the targeted gene. In contrast, the homology directed repair pathway utilizes a repair template to fix the double strand break. Homology directed repair faithfully copies the sequence of the repair template to the cut target sequence. Therefore, specific nucleotide changes are introduced into the targeted gene by the use of homology directed repair with a repair template that incorporates the changes.

Any plant species or plant cell may be genetically modified to comprise a heterologous nucleic acid herein. In some embodiments, the plant cell that is so genetically modified is capable of regeneration to produce a plant. In some embodiments, the plant cell is not capable of regeneration into a plant. In some embodiments, plant cells that are genetically modified (e.g., host plant cells) include cells from, for example and without limitation, a higher plant, a dicotyledonous plant, a monocotyledonous plants, a consumable plant, a crop plant, a plant utilized for its oils (e.g., an oilseed plant), and a non-nodulating plant. Such plants include, for example and without limitation: alfalfa; soybean; cotton; rapeseed (canola); linseed; corn; rice; brachiaria; wheat; safflower; sorghum; sugarbeet; sunflower; tobacco; and grasses (e.g., turf grass).

In particular examples, a genetically modified plant cell or plant herein includes, for example and without limitation: Brassica napus; indian mustard (Brassica juncea); Ethiopian mustard (Brassica carinata); turnip (Brassica rapa); cabbage (Brassica oleracea); Glycine max; Linum usitatissimum; Zea mays; Carthamus tinctorius; Helianthus annuus; Nicotiana tabacum; Arabidopsis thaliana, Brazil nut (Betholettia excelsa); castor bean (Ricinus communis); coconut (Cocus nucifera); coriander (Coriandrum sativum); Gossypium spp.; groundnut (Arachis hypogaea); jojoba (Simmondsia chinensis); oil palm (Elaeis guineeis); olive (Olea eurpaea); Oryza sativa; squash (Cucurbita maxima); barley (Hordeum vulgare); sugarcane (Saccharum officinarum); Triticum spp. (including Triticum durum and Triticum aestivum); and duckweed (Lemnaceae sp.). In some embodiments, the plant may have a particular genetic background, as for elite cultivars, wild-type cultivars, and commercially distinguishable varieties.

According to methods known in the art, nucleic acids can be introduced into essentially any plant. Embodiments herein may employ any of the many methods for the transformation of plants (and production of genetically modified plants) that are known in the art. Such methods include, for example and without limitation, biological and physical transformation protocols for dicotyledenous plants, as well as monocotyledenous plants. See, e.g., Goto-Fumiyuki et al. (1999) Nat. Biotechnol. 17:282; Miki et al. (1993) Methods in Plant Molecular Biology and Biotechnology (Glick, B. R. and Thompson, J. E., Eds.), CRC Press, Inc., Boca Raton, Fla., pp. 67-88. In addition, vectors and in vitro culture methods for plant cell and tissue transformation and regeneration of plants are described, for example, in Gruber and Crosby (1993) Methods in Plant Molecular Biology and Biotechnology, supra, at pp. 89-119.

Plant transformation techniques available for introducing a nucleic acid into a plant host cell include, for example and without limitation: transformation with disarmed T-DNA using Agrobacterium tumefaciens or A. rhizogenes as the transformation agent; calcium phosphate transfection; polybrene transformation; protoplast fusion; electroporation (D'Halluin et al. (1992) Plant Cell 4:1495); ultrasonic methods (e.g., sonoporation); liposome transformation; microinjection; contact with naked DNA; contact with plasmid vectors; contact with viral vectors; biolistics (e.g., DNA particle bombardment (see, e.g., Klein et al. (1987) Nature 327:70) and microparticle bombardment (Sanford et al. (1987) Part. Sci. Technol. 5:27; Sanford (1988) Trends Biotech. 6:299, Sanford (1990) Physiol. Plant 79:206; and Klein et al. (1992) Biotechnology 10:268); silicon carbide WHISKERS-mediated transformation (Kaeppler et al. (1990) Plant Cell Rep. 9:415); nanoparticle transformation (see, e.g., U.S. Patent Publication No. US2009/0104700A1); aerosol beaming; and polyethylene glycol (PEG)-mediated uptake. In specific examples, a heterologous nucleic acid may be introduced directly into the genomic DNA of a plant cell.

A widely utilized method for introducing an expression vector into a plant is based on the natural transformation system of Agrobacterium. Horsch et al. (1985) Science 227:1229. A. tumefaciens and A. rhizogenes are plant pathogenic soil bacteria known to be useful to genetically transform plant cells. The Ti and Ri plasmids of A. tumefaciens and A. rhizogenes, respectively, carry genes responsible for genetic transformation of the plant. Kado (1991) Crit. Rev. Plant. Sci. 10:1. Details regarding Agrobacterium vector systems and methods for Agrobacterium-mediated gene transfer are also available in, for example, Gruber et al., supra, Miki et al., supra, Moloney et al. (1989) Plant Cell Reports 8:238, and U.S. Pat. Nos. 4,940,838 and 5,464,763.

If Agrobacterium is used for the transformation, the DNA to be inserted typically is cloned into special plasmids; either into an intermediate vector or a binary vector. Intermediate vectors cannot replicate themselves in Agrobacterium. The intermediate vector may be transferred into A. tumefaciens by means of a helper plasmid (conjugation). The Japan Tobacco Superbinary system is an example of such a system (reviewed by Komari et al. (2006) Methods in Molecular Biology (K. Wang, ed.) No. 343; Agrobacterium Protocols, 2^{nd} Edition, Vol. 1, Humana Press Inc., Totowa, N.J., pp. 15-41; and Komori et al. (2007) Plant Physiol. 145:1155). Binary vectors can replicate themselves both in E. coli and in Agrobacterium. Binary vectors comprise a selection marker gene and a linker or polylinker which are framed by the right and left T-DNA border regions. They can be transformed directly into Agrobacterium (Holsters, 1978). The Agrobacterium comprises a plasmid carrying a vir region. The Ti or Ri plasmid also comprises the vir region necessary for the transfer of the T-DNA. The vir region is necessary for the transfer of the T-DNA into the plant cell. Additional T-DNA may be contained.

The virulence functions of the Agrobacterium tumefaciens host will direct the insertion of a T-strand containing the construct and adjacent marker into the plant cell DNA when the cell is infected by the bacteria using a binary T DNA vector (Bevan (1984) Nuc. Acid Res. 12:8711) or the co-cultivation procedure (Horsch et al. (1985) Science 227:1229). Generally, the Agrobacterium transformation system is used to engineer dicotyledonous plants. Bevan et al. (1982) Ann. Rev. Genet. 16:357; Rogers et al. (1986) Methods Enzymol. 118:627. The Agrobacterium transformation system may also be used to transform, as well as transfer, nucleic acids to monocotyledonous plants and plant cells. See U.S. Pat. No. 5,591,616; Hemalsteen et al. (1984) EMBO J. 3:3039; Hooykass-Van Slogteren et al. (1984) Nature 311:763; Grimsley et al. (1987) Nature 325:1677; Boulton et al. (1989) Plant Mol. Biol. 12:31; and Gould et al. (1991) Plant Physiol. 95:426.

The genetic manipulations of a recombinant host herein may be performed using standard genetic techniques and screening, and may be carried out in any host cell that is suitable to genetic manipulation. In some embodiments, a recombinant host cell may be any organism or microorganism host suitable for genetic modification and/or recombinant gene expression. In some embodiments, a recombinant host may be a plant. Standard recombinant DNA and molecular cloning techniques used here are well-known in the art and are described in, for example and without limitation: Sambrook et al. (1989), supra; Silhavy et al. (1984) Experiments with Gene Fusions, Cold Spring Harbor Laboratory Press, Cold Spring Harbor, N.Y.; and Ausubel et al. (1987) Current Protocols in Molecular Biology, Greene Publishing Assoc. and Wiley-Interscience, New York, N.Y.

Following the introduction of a nucleic acid into a regeneration-capable plant cell, the plant cell may be grown, and upon emergence of differentiating tissue such as shoots and roots, mature plants can be generated. In some embodiments, a plurality of plants can be generated. Methodologies for regenerating plants are known to those of ordinary skill in the art and can be found, for example, in Plant Cell and Tissue Culture (Vasil and Thorpe, Eds.), Kluwer Academic Publishers, 1994. Genetically modified plants described herein may be cultured in a fermentation medium or grown in a suitable medium such as soil. In some embodiments, a suitable growth medium for higher plants may be any growth medium for plants, including, for example and without limitation; soil, sand, any other particulate media that support root growth (e.g., vermiculite, perlite, etc.) or hydroponic culture, as well as suitable light, water and nutritional supplements that facilitate the growth of the higher plant.

Transformed plant cells which are produced by any of the above transformation techniques can be cultured to regenerate a whole plant that possesses the transformed genotype, and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al. (1983) “Protoplasts Isolation and Culture,” in Handbook of Plant Cell Culture, Macmillian Publishing Company, New York, pp. 124-176; and Binding (1985) Regeneration of Plants, Plant Protoplasts, CRC Press, Boca Raton, pp. 21-73. Regeneration can also be performed from plant callus, explants, organs, pollens, embryos or parts thereof. Such regeneration techniques are described generally in Klee et al. (1987) Ann. Rev. Plant Phys. 38:467.

In embodiments wherein the plant cells that are transformed are not capable of regeneration to produce a plant, such cells may be employed, for example, in developing a plant cell line having a relevant phenotype, for example, increased NUE or decreased nitrogen transporter expression.

A transformed plant cell, callus, tissue or plant may be identified and isolated by selecting or screening the engineered plant material for traits encoded by the marker genes present on the transforming DNA. For instance, selection can be performed by growing the engineered plant material on media containing an inhibitory amount of the antibiotic or herbicide to which the transforming gene construct confers resistance. Further, transformed plants and plant cells can also be identified by screening for the activities of any visible marker genes (e.g., the β-glucuronidase, luciferase, or gfp genes) that may be present on the recombinant nucleic acid constructs. Such selection and screening methodologies are well known to those skilled in the art.

A transgenic plant containing a heterologous molecule herein can be produced through selective breeding, for example, by sexually crossing a first parental plant comprising the molecule, and a second parental plant, thereby producing a plurality of first progeny plants. A first progeny plant may then be selected that is resistant to a selectable marker (e.g., glyphosate, resistance to which may be conferred upon the progeny plant by the heterologous molecule herein). The first progeny plant may then by selfed, thereby producing a plurality of second progeny plants. Then, a second progeny plant may be selected that is resistant to the selectable marker. These steps can further include the back-crossing of the first progeny plant or the second progeny plant to the second parental plant or a third parental plant.

It is also to be understood that two different transgenic plants can also be mated to produce offspring that contain two independently segregating, added, exogenous genes. Selfing of appropriate progeny can produce plants that are homozygous for both added, exogenous genes. Back-crossing to a parental plant and out-crossing with a non-transgenic plant are also contemplated, as is vegetative propagation. Other breeding methods commonly used for different traits and crops are known in the art. Backcross breeding has been used to transfer genes for a simply inherited, highly heritable trait into a desirable homozygous cultivar or inbred line, which is the recurrent parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

A nucleic acid may also be introduced into a predetermined area of the plant genome through homologous recombination. Methods to stably integrate a polynucleotide sequence within a specific chromosomal site of a plant cell via homologous recombination have been described within the art. For instance, site specific integration as described in U.S. Patent Publication No. 2009/0111188 A1 involves the use of recombinases or integrases to mediate the introduction of a donor polynucleotide sequence into a chromosomal target. In addition, PCT International Patent Publication No. WO 2008/021207 describes zinc finger mediated-homologous recombination to stably integrate one or more donor polynucleotide sequences within specific locations of the genome. The use of recombinases such as FLP/FRT as described in U.S. Pat. No. 6,720,475, or CRE/LOX as described in U.S. Pat. No. 5,658,772, can be utilized to stably integrate a polynucleotide sequence into a specific chromosomal site. Finally, the use of meganucleases for targeting donor polynucleotides into a specific chromosomal location is described in Puchta et al. (1996) Proc. Natl. Acad. Sci. USA 93:5055.

Other various methods for site specific integration within plant cells are generally known and applicable. Kumar et al. (2001) Trends Plant Sci. 6(4):155. Furthermore, site-specific recombination systems that have been identified in several prokaryotic and lower eukaryotic organisms may be applied for use in plants. Examples of such systems include, but are not limited too; the R/RS recombinase system from the pSR1 plasmid of the yeast Zygosaccharomyces rouxii (Araki et al. (1985) J. Mol. Biol. 182:191), and the Gin/gix system of phage Mu (Maeser and Kahlmann (1991) Mol. Gen. Genet. 230:170).

Site-specific integration techniques may be employed in certain embodiments herein, for example and without limitation, to replace a BT1 and/or BT2 gene with a gene knockout or mutation; e.g., a polynucleotide wherein the coding sequence has been removed, or wherein one or more operably linked regulatory sequences have been removed or altered.

Various assays can be employed in connection with the nucleic acid molecule of certain embodiments herein. In addition to phenotypic observations, the following techniques are useful in detecting the presence of a nucleic acid molecule in a plant cell. For example, the presence of the molecule can be determined by using a primer or probe of the sequence, an ELISA assay to detect an encoded protein, a Western blot to detect the protein, or a Northern or Southern blot to detect RNA or DNA. Additional techniques, such as in situ hybridization, enzyme staining, and immunostaining, also may be used to detect the presence or expression of a recombinant construct in specific plant organs and tissues.

Southern analysis is a commonly used detection method, wherein DNA is cut with restriction endonucleases and fractionated on an agarose gel to separate the DNA by molecular weight and then transferring to nylon membranes. It is then hybridized with the probe fragment which was radioactively labeled with ³²P (or other probe labels) and washed in an SDS solution.

Likewise, Northern analysis deploys a similar protocol, wherein RNA is cut with restriction endonucleases and fractionated on an agarose gel to separate the RNA by molecular weight and then transferring to nylon membranes. It is then hybridized with the probe fragment which was radioactively labeled with ³²P (or other probe labels) and washed in an SDS solution. Analysis of the RNA (e.g., mRNA) isolated from the tissues of interest can indicate relative expression levels. Typically, if the mRNA is present or the amount of mRNA has increased, it can be assumed that the corresponding transgene is being expressed. Northern analysis, or other mRNA analytical protocols, can be used to determine expression levels of an introduced transgene or native gene.

Nucleic acids herein, or segments thereof, may be used to design primers for PCR amplification. In performing PCR amplification, a certain degree of mismatch can be tolerated between primer and template. Mutations, insertions, and deletions can be produced in a given primer by methods known to an ordinarily skilled artisan.

Hydrolysis probe assay, otherwise known as TAQMAN® (Life Technologies, Foster City, Calif.), is another method of detecting and quantifying the presence of a DNA sequence. Briefly, a FRET oligonucleotide probe is designed with one oligo within the transgene and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization.

### VI. Plants, Plant Parts, and Plant Materials Comprising a BTB Knockout or Mutation

Some embodiments herein provide plants comprising at least one heterologous BT1 and/or BT2 polynucleotide knockout event, mutant bt1 and/or bt2 gene, and/or polynucleotide encoding an iRNA targeting a BT1 and/or BT2 gene, such as may be regenerated from stably transformed plant cells or tissues, or may be produced by introgression of such a nucleic acid from a donor line. Such plants may be used or cultivated in any manner, wherein presence of the transforming polynucleotide(s) of interest is desirable. Accordingly, transgenic plants may be engineered to, inter alia, have one or more desired traits (e.g., increased NUE), by transformation, and then may be cropped and cultivated by any method known to those of skill in the art. Particular embodiments herein provide parts, cells, and/or tissues of such transgenic plants. Plant parts, without limitation, include seed, endosperm, ovule and pollen. In some embodiments, the plant part is a seed.

Representative, non-limiting example plants include non-nodulating plants; Arabidopsis; field crops (e.g. alfalfa, barley, bean, clover, corn, cotton, flax, lentils, maize, pea, rape/canola, rice, rye, safflower, sorghum, soybean, sunflower, tobacco, and wheat); vegetable crops (e.g., asparagus, beet, Brassica, broccoli, Brussels sprouts, cabbage, carrot, cauliflower, celery, cucumber (cucurbits), eggplant, lettuce, mustard, onion, pepper, potato, pumpkin, radish, spinach, squash, taro, tomato, and zucchini); fruit and nut crops (e.g., almond, apple, apricot, banana, blackberry, blueberry, cacao, cassava, cherry, citrus, coconut, cranberry, date, hazelnut, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon); tree woods and ornamentals (e.g., alder, ash, aspen, azalea, birch, boxwood, camellia, carnation, chrysanthemum, elm, fir, ivy, jasmine, juniper, oak, palm, poplar, pine, redwood, rhododendron, rose, and rubber).

To confirm the presence of a heterologous polynucleotide(s) of interest in a regenerating plant, a variety of assays may be performed. Such assays include, for example and without limitation: biochemical assays, such as detecting the presence of a protein product, e.g., by immunological means (ELISA and/or Western blots) or by enzymatic function; plant part assays (e.g., leaf or root assays); and analysis of the phenotype of the plant.

There are numerous steps in the development of any novel, desirable plant germplasm, which may begin with the generation of a transgenic crop plant. In some embodiments, a transgenic plant comprising at least one BT1 and/or BT2 polynucleotide knockout event, mutant bt1 and/or bt2 gene, and/or polynucleotide encoding an iRNA targeting a BT1 and/or BT2 gene may be used in a plant breeding and/or germplasm development program.

Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety an improved combination of desirable traits from the parental germplasm. These important traits may include increased NUE, higher seed yield, resistance to diseases and insects, better stems and roots, tolerance to drought and heat, and better agronomic quality.

The choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods include pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to appropriate standards in environments representative of the commercial target area(s) for three or more years. The best lines are candidates for new commercial cultivars; those still deficient in a few traits may be used as parents to produce new populations for further selection.

These processes, which lead to the final step of marketing and distribution, usually take from eight to twelve years from the time the first cross is made. Therefore, development of new cultivars is a time-consuming process that requires precise forward planning, efficient use of resources, and a minimum of changes in direction.

Pedigree breeding and recurrent selection breeding methods are used to develop cultivars from breeding populations. Breeding programs combine desirable traits from two or more cultivars or various broad-based sources into breeding pools from which cultivars are developed by selfing and selection of desired phenotypes. The new cultivars are evaluated to determine which have commercial potential.

Pedigree breeding is used commonly for the improvement of self-pollinating crops. Two parents which possess favorable, complementary traits are crossed to produce an F₁. An F₂ population is produced by selfing one or several F₁s. Selection of the best individuals may begin in the F₂ population; then, beginning in the F₃, the best individuals in the best families are selected. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In embodiments herein, at least one BT1 and/or BT2 polynucleotide knockout event, mutant bt1 and/or bt2 gene, and/or polynucleotide encoding an iRNA targeting a BT1 and/or BT2 gene may be introduced into a plant germplasm, for example, to develop novel inbred lines that are characterized by increased NUE, under the control of regulatory elements that are operably linked to the polynucleotide(s). A particular advantage of such a development program may be that the expression of an increased NUE phenotype results in, for example, increased growth under N limiting conditions.

The following examples are provided to illustrate certain particular features and/or embodiments. The examples should not be construed to limit the disclosure to the particular features or embodiments exemplified.

### EXAMPLES

### Example 1: Materials and Methods

New target genes directly modulating NUE in planta were discovered by using a systems approach in the model plant system, Arabidopsis. Gutierrez (2012) Science 336(6089):1673-5.

The systems approach comprised four steps: (I) data integration, (2) modeling, (3) hypothesis generation, and (4) experimental validation. Gutiérrez et al. (2005) Plant Physiol. 138(2):550-4. The first three steps with the Discriminative Local Subspaces (DLS) algorithm were carried out. Puelma et al. (2012) Bioinformatics 28(17):2256-64. DLS generated a gene network with a potential role in NUE.

This model was used to predict that BT2 is a central gene for NUE. BT2 is a member of the BTB family of scaffold proteins (Gingerich et al. (2007) Plant Cell 19(8):2329-48), known to play a crucial role in both male and female gametophyte development (Robert et al. (2009) Plant J. Cell. Mol. Biol. 58(1):109-21. BTB2 can activate telomerase expression in mature Arabidopsis leaves. Ren et al. (2007) Plant Cell 19(1):23-31. Moreover, BT2 gene expression is regulated by a number of signals including circadian regulation, sugar and nitrogen nutrients, hormones, cold, hydrogen peroxide, and wounding stress treatments. Mandadi et al. (2009) Plant Physiol. 150(4):1930-9.

A key role for BTB2 in NUE, a complex trait that results of integrating environmental and internal signals over the life cycle of the plant, was demonstrated for the first time. Overexpression of BTB2 reduces NUE, negatively affects primary root growth, and lowers plant biomass, as compared to wild-type plants under low nitrate conditions. In contrast, mutant bt2 plants with a second mutation in the closest bt2 homolog, bt1, exhibited the opposite phenotype.

These results indicate that BTB1 and BTB2 are negative determinants of plant NUE and growth under low nitrate conditions. BTB1 and BTB2 were found to negatively affect nitrate uptake by down-regulating major components of the high affinity nitrate transport system. This results evidence that BTB proteins are part of a conserved mechanism controlling growth and NUE under N-limiting conditions in dicotyledonous plants.

Plant Material and Plants Growth Conditions.

Arabidopsis thaliana Columbia-0 (Col-0), and a crossing between Col-0 and Landsberg erecta (Ler), were used as wild-type backgrounds, as indicated. bt1-4 was obtained from the Arabidopsis Biological Resource Center mutant bank (located on the World Wide Web at arabidopsis.org), bt2-1 and a BT2 over-expressor line (BT2OE) were kindly donated.

Arabidopsis were grown in an inert substrate, vermiculite, under long-day (16 h light at 120 μmol·m⁻²s⁻¹/8 h dark) conditions at 22° C. in plant growth incubators (Percival Scientific, Iowa, U.S.). Plants were watered every week with 200 mL medium containing 50 μM H₃BO₃, 1.5 mM CaCl₂, 50 μM MnSO₄, 0.08 μM CuSO₄, 0.05 μM Na₂MoO₄, 0.625 mM KH₂PO₄, 0.75 mM MgSO₄, 25 μM ZnSO₄, 5 μM KI, 50 CpM FeSO₄, 50 μM Na₂EDTA, and 0.055 μM CoCl₂, supplemented with different amounts of nitrate in the form of KNO₃: 0.5 mM KNO₃; 1 mM KNO₃; 5 mM KNO₃; 10 mM KNO₃; 20 mM KNO₃ and 30 mM KNO₃, pH: 5.7, until the plants completed their life cycle.

To evaluate gene expression in 15 day-old plants, plants were grown in 50 mL 0.8% agar vertical plates with the medium previously described, supplemented with 0.5 mM or 5 mM KNO₃.

NUE and Biomass Measurement.

To evaluate NUE, the agronomic index, defined as the number of seeds per plant (gr)/applied N throughout plant life cycle (gr), was used. Mol et al. (1982) Agron. J. 74:562-4. Biomass was measured as dry weight (DW) of plants after incubating at 70° C. for 48 hours.

GENIUS Network Prediction for NUE.

To identify relevant genes for NUE, a gene network was inferred using the DLS tool for MATLAB. Puelma et al. (2012), supra. Expression data matrix and gene annotations were used as inputs. The expression data matrix contained 3,911 features from 2,017 Affymetrix chips. The gene annotations correspond to Gene Ontology annotations from Sep. 7, 2010. As input to DLS 12 biological processes were selected from Gene Ontology related to NUE, totaling 220 genes. Six of these processes are associated with N metabolism (nitrate assimilation GO:0042128, nitrate transport GO:0015706, ammonium transport GO:0015696, ammonium response GO:0060359, nitrogen response GO:0019740, nitrate response GO:0010167), while the other six are associated with development (regulation of seed development GO:0080050, organ senescence GO:0010260, endosperm development GO:0009960, vegetative to reproductive phase transition of meristem GO:0010228, vegetative phase change GO:0010050, seed maturation GO:0010431). Cytoscape (Lopes et al. (2010) Bioinformatics 26(18):2347-8) was used to analyze the inferred network and the “Network Analyzer” plugin was applied to calculate the degree and betweenness centrality of each node. A network view was constructed in which nodes have sizes that are proportional to their degrees and were colored according to their betweenness centrality values. The complete network contains a total of 350 genes. Table S1.

TABLE S1
350 genes obtained by the DLS method.

| | Betweenness | | |
|---|---|---|---|
| Locus of gene | centrality value | Degree value | Final ordering score |
| AT3G48360 | 0.3192 | 71 | 0.3192187 |
| AT1G54040 | 0.14914522 | 16 | 0.03361019 |
| AT2G15620 | 0.1297914 | 62 | 0.113338969 |
| AT2G40170 | 0.1180321 | 26 | 0.043223023 |
| AT2G29090 | 0.11371784 | 7 | 0.01121162 |
| AT1G12820 | 0.11169879 | 13 | 0.02045189 |
| AT1G78050 | 0.10654633 | 63 | 0.09454111 |
| AT2G01520 | 0.10273314 | 10 | 0.01446946 |
| AT2G21660 | 0.1027 | 56 | 0.08096568 |
| AT1G08090 | 0.09824827 | 42 | 0.05811869 |
| AT1G77760 | 0.08513516 | 59 | 0.07074612 |
| AT5G53460 | 0.08385167 | 48 | 0.05668845 |
| AT3G27170 | 0.07525176 | 24 | 0.02543721 |
| AT1G48130 | 0.07242205 | 34 | 0.03468098 |
| AT1G69490 | 0.0708 | 8 | 0.00798204 |
| AT5G64930 | 0.06746025 | 7 | 0.00665101 |
| AT1G79000 | 0.0653 | 6 | 0.00552178 |
| AT5G50200 | 0.06236793 | 47 | 0.04128581 |
| AT2G40080 | 0.0599 | 49 | 0.04137017 |
| AT1G37130 | 0.05695835 | 35 | 0.02807806 |
| AT1G12110 | 0.0522532 | 47 | 0.03459015 |
| AT1G08100 | 0.05074448 | 12 | 0.00857653 |
| AT2G17820 | 0.04904711 | 24 | 0.0165793 |
| AT5G39610 | 0.0480 | 8 | 0.00540632 |
| AT1G55350 | 0.04719938 | 3 | 0.00199434 |
| AT1G05620 | 0.04633135 | 3 | 0.00195766 |
| AT1G25560 | 0.04486276 | 11 | 0.00695057 |
| AT3G22420 | 0.03942298 | 4 | 0.00222101 |
| AT1G54060 | 0.0394 | 2 | 0.00111051 |
| AT2G46830 | 0.0386 | 55 | 0.02987554 |
| AT4G21680 | 0.03730284 | 5 | 0.00262696 |
| AT2G15790 | 0.03410401 | 5 | 0.00240169 |
| AT1G12940 | 0.03404167 | 12 | 0.00575352 |
| AT1G73680 | 0.03388993 | 2 | 0.00095465 |
| AT5G63980 | 0.0323 | 5 | 0.00227563 |
| AT1G03880 | 0.03228835 | 22 | 0.01000484 |
| AT2G25930 | 0.0313 | 29 | 0.01277811 |
| AT2G35980 | 0.02949189 | 4 | 0.00166151 |
| AT2G33860 | 0.0290 | 4 | 0.00163589 |
| AT1G69870 | 0.02850269 | 6 | 0.00240868 |
| AT5G02030 | 0.0283 | 2 | 0.00079786 |
| AT3G07650 | 0.0273 | 50 | 0.01924563 |
| AT1G22770 | 0.0251 | 7 | 0.00247309 |
| AT4G32980 | 0.0249 | 6 | 0.00210791 |
| AT1G01060 | 0.0243 | 42 | 0.01436968 |
| AT5G63110 | 0.0231 | 3 | 0.00097772 |
| AT3G52430 | 0.0227 | 2 | 0.00064014 |
| AT5G40890 | 0.02074437 | 16 | 0.00467479 |
| AT5G62640 | 0.01833605 | 2 | 0.00051651 |
| AT3G63210 | 0.01798297 | 5 | 0.00126641 |
| AT5G40010 | 0.01714257 | 4 | 0.00096578 |
| AT3G57920 | 0.0171 | 3 | 0.00072364 |
| AT4G19230 | 0.01709317 | 2 | 0.0004815 |
| AT4G02280 | 0.01707148 | 2 | 0.00048089 |
| AT5G11520 | 0.01603604 | 5 | 0.0011293 |
| AT5G37600 | 0.01460605 | 11 | 0.00226291 |
| AT4G01250 | 0.0139 | 7 | 0.00137183 |
| AT1G12910 | 0.0133 | 3 | 0.00056285 |
| AT2G38290 | 0.01310872 | 7 | 0.00129241 |
| AT4G28520 | 0.01274736 | 19 | 0.00341127 |
| AT5G44610 | 0.0119 | 4 | 0.00067246 |
| AT5G14570 | 0.01144485 | 4 | 0.00064478 |
| AT2G17660 | 0.01144485 | 3 | 0.00048359 |
| AT2G39770 | 0.01142838 | 2 | 0.00032193 |
| AT4G25420 | 0.0098 | 3 | 0.0004152 |
| AT4G23810 | 0.0096 | 7 | 0.00094843 |
| AT1G64780 | 0.00908851 | 20 | 0.00256014 |
| AT5G12470 | 0.00773584 | 3 | 0.00032687 |
| AT1G66200 | 0.00751254 | 17 | 0.00179878 |
| AT3G21060 | 0.00696494 | 2 | 0.0001962 |
| AT4G24400 | 0.00603509 | 2 | 0.00017 |
| AT1G03890 | 0.0059792 | 7 | 0.0005895 |
| AT5G13170 | 0.00573066 | 3 | 0.00024214 |
| AT5G54650 | 0.00573066 | 3 | 0.00024214 |
| AT2G01570 | 0.0057 | 3 | 0.00024214 |
| AT3G20740 | 0.0057 | 2 | 0.00016143 |
| AT2G39730 | 0.00573066 | 2 | 0.00016143 |
| AT1G27650 | 0.00573066 | 2 | 0.00016143 |
| AT5G60450 | 0.0057 | 2 | 0.00016143 |
| AT2G42200 | 0.0057 | 2 | 0.00016143 |
| AT5G45900 | 0.00533531 | 2 | 0.00015029 |
| AT5G37770 | 0.0038 | 4 | 0.00021191 |
| AT1G02860 | 0.0038 | 3 | 0.00015849 |
| AT1G18880 | 0.00359308 | 4 | 0.00020243 |
| AT1G49500 | 0.00353201 | 7 | 0.00034823 |
| AT3G17820 | 0.00335328 | 2 | 9.4459E−05 |
| AT5G40850 | 0.00321208 | 10 | 0.00045241 |
| AT5G44120 | 0.00291581 | 16 | 0.00065708 |
| AT2G28550 | 0.0029 | 5 | 0.00020407 |
| AT1G65480 | 0.0029 | 3 | 0.00012055 |
| AT5G62720 | 0.00207694 | 4 | 0.00011701 |
| AT5G03280 | 0.0017 | 2 | 4.6804E−05 |
| AT1G27320 | 0.0017 | 2 | 4.6634E−05 |
| AT4G32950 | 0.00156295 | 3 | 6.604E−05 |
| AT2G21070 | 0.00152906 | 4 | 8.6144E−05 |
| AT1G25550 | 0.00130554 | 6 | 0.00011033 |
| AT2G41280 | 0.00115375 | 12 | 0.000195 |
| AT3G29035 | 0.00112892 | 4 | 6.3601E−05 |
| AT1G69440 | 0.0008 | 2 | 2.2586E−05 |
| AT4G38340 | 5.02E−04 | 2 | 1.4154E−05 |
| AT5G65010 | 4.49E−04 | 4 | 2.5321E−05 |
| AT5G40420 | 3.99E−04 | 6 | 3.3697E−05 |
| AT5G16570 | 3.87E−04 | 6 | 3.2702E−05 |
| AT5G54740 | 3.60E−04 | 6 | 3.0442E−05 |
| AT3G22640 | 3.37E−04 | 5 | 2.3722E−05 |
| AT1G30510 | 2.48E−04 | 10 | 3.4923E−05 |
| AT5G13110 | 2.48E−04 | 10 | 3.4923E−05 |
| AT1G32450 | 2.24E−04 | 3 | 9.4475E−06 |
| AT4G13510 | 2.11E−04 | 4 | 1.1897E−05 |
| AT1G24280 | 2.10E−04 | 9 | 2.662E−05 |
| AT5G41670 | 2.10E−04 | 9 | 2.662E−05 |
| AT2G22450 | 2.04E−04 | 9 | 2.5901E−05 |
| AT4G33980 | 2.04E−04 | 8 | 2.3023E−05 |
| AT1G64190 | 1.72E−04 | 7 | 1.7E−05 |
| AT5G61380 | 1.27E−04 | 8 | 1.4345E−05 |
| AT5G48250 | 1.27E−04 | 8 | 1.4345E−05 |
| AT4G38960 | 1.27E−04 | 8 | 1.4345E−05 |
| AT5G42900 | 1.27E−04 | 8 | 1.4345E−05 |
| AT1G28050 | 1.27E−04 | 8 | 1.4345E−05 |
| AT3G12320 | 1.27E−04 | 7 | 1.2552E−05 |
| AT4G16146 | 1.27E−04 | 7 | 1.2552E−05 |
| AT3G54500 | 1.27E−04 | 6 | 1.0759E−05 |
| AT5G60770 | 1.15E−04 | 4 | 6.5014E−06 |
| AT1G63940 | 1.14E−04 | 8 | 1.2791E−05 |
| AT4G05390 | 1.14E−04 | 8 | 1.2791E−05 |
| AT2G14210 | 9.30E−05 | 2 | 2.6192E−06 |
| AT1G43710 | 7.60E−05 | 6 | 6.4183E−06 |
| AT1G73190 | 3.93E−05 | 4 | 2.2141E−06 |
| AT1G14340 | 3.76E−05 | 6 | 3.1749E−06 |
| AT2G26980 | 3.57E−05 | 7 | 3.5177E−06 |
| AT3G48990 | 3.57E−05 | 5 | 2.5127E−06 |
| AT2G28490 | 8.23E−06 | 4 | 4.6366E−07 |
| AT5G13420 | 0 | 8 | 0 |
| AT1G17665 | 0 | 7 | 0 |
| AT2G27510 | 0 | 7 | 0 |
| AT1G79440 | 0 | 6 | 0 |
| AT4G09620 | 0 | 6 | 0 |
| AT5G42990 | 0 | 6 | 0 |
| AT3G16560 | 0 | 6 | 0 |
| AT5G39410 | 0 | 6 | 0 |
| AT5G57110 | 0 | 5 | 0 |
| AT2G47490 | 0 | 5 | 0 |
| AT3G20810 | 0 | 5 | 0 |
| AT5G67420 | 0 | 5 | 0 |
| AT4G15430 | 0 | 5 | 0 |
| AT4G25140 | 0 | 5 | 0 |
| AT3G02380 | 0 | 5 | 0 |
| AT2G19450 | 0 | 5 | 0 |
| AT2G31380 | 0 | 5 | 0 |
| AT1G13300 | 0 | 5 | 0 |
| AT5G60100 | 0 | 5 | 0 |
| AT2G39200 | 0 | 4 | 0 |
| AT2G21320 | 0 | 4 | 0 |
| AT1G07050 | 0 | 4 | 0 |
| AT1G54870 | 0 | 4 | 0 |
| AT5G06980 | 0 | 4 | 0 |
| AT5G23240 | 0 | 4 | 0 |
| AT4G26970 | 0 | 4 | 0 |
| AT1G19370 | 0 | 4 | 0 |
| AT5G14550 | 0 | 4 | 0 |
| AT1G49860 | 0 | 4 | 0 |
| AT4G26670 | 0 | 4 | 0 |
| AT3G19030 | 0 | 4 | 0 |
| AT4G27130 | 0 | 4 | 0 |
| AT5G39590 | 0 | 4 | 0 |
| AT3G01570 | 0 | 4 | 0 |
| AT2G21130 | 0 | 4 | 0 |
| AT4G24620 | 0 | 4 | 0 |
| AT1G27630 | 0 | 4 | 0 |
| AT1G70410 | 0 | 4 | 0 |
| AT2G02760 | 0 | 3 | 0 |
| AT3G14940 | 0 | 3 | 0 |
| AT4G30190 | 0 | 3 | 0 |
| AT3G55450 | 0 | 3 | 0 |
| AT4G32340 | 0 | 3 | 0 |
| AT3G49940 | 0 | 3 | 0 |
| AT2G42070 | 0 | 3 | 0 |
| AT1G73920 | 0 | 3 | 0 |
| AT4G27160 | 0 | 3 | 0 |
| AT5G64940 | 0 | 3 | 0 |
| AT4G25350 | 0 | 3 | 0 |
| AT2G25730 | 0 | 3 | 0 |
| AT5G66400 | 0 | 3 | 0 |
| AT5G12860 | 0 | 3 | 0 |
| AT2G41250 | 0 | 3 | 0 |
| AT3G60750 | 0 | 3 | 0 |
| AT3G23280 | 0 | 3 | 0 |
| AT3G27660 | 0 | 3 | 0 |
| AT1G68670 | 0 | 3 | 0 |
| AT5G06690 | 0 | 3 | 0 |
| AT4G20400 | 0 | 2 | 0 |
| AT3G56350 | 0 | 2 | 0 |
| AT2G21490 | 0 | 2 | 0 |
| AT5G10030 | 0 | 2 | 0 |
| AT3G09390 | 0 | 2 | 0 |
| AT1G14920 | 0 | 2 | 0 |
| AT1G07610 | 0 | 2 | 0 |
| AT1G20780 | 0 | 2 | 0 |
| AT5G11150 | 0 | 2 | 0 |
| AT3G17800 | 0 | 2 | 0 |
| AT2G21820 | 0 | 2 | 0 |
| AT2G24540 | 0 | 2 | 0 |
| AT4G04330 | 0 | 2 | 0 |
| AT1G17020 | 0 | 2 | 0 |
| AT4G26740 | 0 | 2 | 0 |
| AT1G64530 | 0 | 2 | 0 |
| AT1G17810 | 0 | 2 | 0 |
| AT3G03450 | 0 | 2 | 0 |
| AT4G27140 | 0 | 2 | 0 |
| AT2G43760 | 0 | 2 | 0 |
| AT4G33700 | 0 | 2 | 0 |
| AT1G05510 | 0 | 2 | 0 |
| AT2G16060 | 0 | 2 | 0 |
| AT2G29700 | 0 | 2 | 0 |
| AT1G16170 | 0 | 2 | 0 |
| AT4G37540 | 0 | 2 | 0 |
| AT2G15890 | 0 | 2 | 0 |
| AT1G74030 | 0 | 2 | 0 |
| AT5G23730 | 0 | 2 | 0 |
| AT3G21890 | 0 | 2 | 0 |
| AT2G22480 | 0 | 2 | 0 |
| AT1G27080 | 0 | 1 | 0 |
| AT1G07180 | 0 | 1 | 0 |
| AT5G14760 | 0 | 1 | 0 |
| AT4G31570 | 0 | 1 | 0 |
| AT5G62910 | 0 | 1 | 0 |
| AT3G50500 | 0 | 1 | 0 |
| AT1G23870 | 0 | 1 | 0 |
| AT4G19240 | 0 | 1 | 0 |
| AT3G54940 | 0 | 1 | 0 |
| AT4G25835 | 0 | 1 | 0 |
| AT3G17520 | 0 | 1 | 0 |
| AT4G16160 | 0 | 1 | 0 |
| AT1G78000 | 0 | 1 | 0 |
| AT5G45890 | 0 | 1 | 0 |
| AT3G15010 | 0 | 1 | 0 |
| AT5G26880 | 0 | 1 | 0 |
| AT1G20900 | 0 | 1 | 0 |
| AT2G32950 | 0 | 1 | 0 |
| AT2G46240 | 0 | 1 | 0 |
| AT4G08930 | 0 | 1 | 0 |
| AT5G04590 | 0 | 1 | 0 |
| AT4G02380 | 0 | 1 | 0 |
| AT2G41060 | 0 | 1 | 0 |
| AT3G19190 | 0 | 1 | 0 |
| AT2G45650 | 0 | 1 | 0 |
| AT4G36010 | 0 | 1 | 0 |
| AT5G65110 | 0 | 1 | 0 |
| AT3G50980 | 0 | 1 | 0 |
| AT1G04010 | 0 | 1 | 0 |
| AT1G56660 | 0 | 1 | 0 |
| AT1G52990 | 0 | 1 | 0 |
| AT2G22400 | 0 | 1 | 0 |
| AT3G13930 | 0 | 1 | 0 |
| AT1G58080 | 0 | 1 | 0 |
| AT3G01090 | 0 | 1 | 0 |
| AT2G01830 | 0 | 1 | 0 |
| AT2G25890 | 0 | 1 | 0 |
| AT5G35630 | 0 | 1 | 0 |
| AT1G49160 | 0 | 1 | 0 |
| AT2G23240 | 0 | 1 | 0 |
| AT1G60220 | 0 | 1 | 0 |
| AT3G07350 | 0 | 1 | 0 |
| AT5G40645 | 0 | 1 | 0 |
| AT1G03160 | 0 | 1 | 0 |
| AT1G56300 | 0 | 1 | 0 |
| AT1G01040 | 0 | 1 | 0 |
| AT4G09020 | 0 | 1 | 0 |
| AT5G62430 | 0 | 1 | 0 |
| AT3G52360 | 0 | 1 | 0 |
| AT5G51640 | 0 | 1 | 0 |
| AT5G07190 | 0 | 1 | 0 |
| AT2G15010 | 0 | 1 | 0 |
| AT2G35510 | 0 | 1 | 0 |
| AT2G35300 | 0 | 1 | 0 |
| AT1G61740 | 0 | 1 | 0 |
| AT5G45830 | 0 | 1 | 0 |
| AT5G02810 | 0 | 1 | 0 |
| AT2G35230 | 0 | 1 | 0 |
| AT5G17880 | 0 | 1 | 0 |
| AT1G10570 | 0 | 1 | 0 |
| AT5G45380 | 0 | 1 | 0 |
| AT2G30670 | 0 | 1 | 0 |
| AT3G26640 | 0 | 1 | 0 |
| AT1G02700 | 0 | 1 | 0 |
| AT2G22630 | 0 | 1 | 0 |
| AT3G06110 | 0 | 1 | 0 |
| AT5G62490 | 0 | 1 | 0 |
| AT5G42820 | 0 | 1 | 0 |
| AT3G45060 | 0 | 1 | 0 |
| AT4G27150 | 0 | 1 | 0 |
| AT1G15670 | 0 | 1 | 0 |
| AT2G36390 | 0 | 1 | 0 |
| AT5G55850 | 0 | 1 | 0 |
| AT1G80480 | 0 | 1 | 0 |
| AT1G54860 | 0 | 1 | 0 |
| AT1G60140 | 0 | 1 | 0 |
| AT2G46650 | 0 | 1 | 0 |
| AT5G65210 | 0 | 1 | 0 |
| AT5G62520 | 0 | 1 | 0 |
| AT2G20610 | 0 | 1 | 0 |
| AT2G18390 | 0 | 1 | 0 |
| AT2G48080 | 0 | 1 | 0 |
| AT5G08010 | 0 | 1 | 0 |
| AT2G47770 | 0 | 1 | 0 |
| AT1G51450 | 0 | 1 | 0 |
| AT3G47860 | 0 | 1 | 0 |
| AT3G15670 | 0 | 1 | 0 |
| AT5G18540 | 0 | 1 | 0 |
| AT4G24670 | 0 | 1 | 0 |
| AT1G32900 | 0 | 1 | 0 |
| AT4G09600 | 0 | 1 | 0 |
| AT4G02020 | 0 | 1 | 0 |
| AT5G35970 | 0 | 1 | 0 |
| AT2G33150 | 0 | 1 | 0 |
| AT4G26460 | 0 | 1 | 0 |
| AT1G31230 | 0 | 1 | 0 |
| AT4G14270 | 0 | 1 | 0 |
| AT5G53240 | 0 | 1 | 0 |
| AT5G45690 | 0 | 1 | 0 |
| AT5G15360 | 0 | 1 | 0 |
| AT2G38560 | 0 | 1 | 0 |
| AT5G17290 | 0 | 1 | 0 |
| AT4G38680 | 0 | 1 | 0 |
| AT5G24120 | 0 | 1 | 0 |
| AT2G42350 | 0 | 1 | 0 |
| AT1G26665 | 0 | 1 | 0 |
| AT5G10210 | 0 | 1 | 0 |
| AT1G80380 | 0 | 1 | 0 |
| AT4G27170 | 0 | 1 | 0 |
| AT1G62180 | 0 | 1 | 0 |
| AT5G19970 | 0 | 1 | 0 |
| AT3G05260 | 0 | 1 | 0 |
| AT5G35750 | 0 | 1 | 0 |
| AT3G05880 | 0 | 1 | 0 |
| AT4G31910 | 0 | 1 | 0 |
| AT1G72100 | 0 | 1 | 0 |
| AT1G73870 | 0 | 1 | 0 |
| AT5G10820 | 0 | 1 | 0 |
| AT2G41260 | 0 | 1 | 0 |
| AT5G20990 | 0 | 1 | 0 |
| AT1G69040 | 0 | 1 | 0 |
| AT5G13020 | 0 | 1 | 0 |
| AT4G31550 | 0 | 1 | 0 |
| AT4G24210 | 0 | 1 | 0 |
| AT2G26600 | 0 | 1 | 0 |
| AT5G44720 | 0 | 1 | 0 |
| AT5G51570 | 0 | 1 | 0 |
| AT5G20320 | 0 | 1 | 0 |
| AT1G04560 | 0 | 1 | 0 |

Nitrate Uptake into the Shoot.

Col-0 and OEBT2, Col-0×Ler, and bt1bt2 plants were grown under the same experimental conditions described above in the vermiculite substrate. Net NO₃⁻ uptake was measured by treating plants at dawn on day 30 when plants were still in the vegetative stage. Treatment consisted of replacing the nutrient solution by an ¹⁵N-containing solution that had the same nutrient composition (0.5 mM or 5 mM KNO₃, and 10% ¹⁵N (w/w) enrichment). Pots were maintained in this solution for 24 hours. Rosettes were cut, washed for 1 min in 0.1 mM CaSO₄, and were then dried at 70° C. for 48 hours, and their dry weight (DW) was determined. Total ¹⁵NO₃ content was evaluated using an ANCA-MS system (Europa Scientific, Cambridge, UK). Clarkson et al. (1996) Plant Cell Environ. 19:859-68. Net uptake of NO₃⁻ for each genotype was calculated from the total ¹⁵N content of plants.

RNA Isolation and qRT-PCR.

RNA was isolated from whole plants or root and shoot tissues as indicated. RNA extraction was performed with the Pure Link RNA mini kit according to the manufacturer's instructions (Life Technologies, California, U.S.). cDNA synthesis was carried out using the IMPROM-II reverse transcriptase according to the manufacturer's instructions (Promega, Wis., U.S.). qRT-PCR was carried out using the BRILLIANT III Ultra-Fast SYBR Green QPCR Reagents on a StepOne Real Time system (Life technologies, California, U.S.). RNA levels were normalized relative to ADAPTOR PROTEIN-4 MU-ADAPTIN (At4g24550).

Evaluation of Stage Development Changes in Vegetative Growth.

BT2OEX and bt1bt2, and their corresponding WT plants, were grown in vermiculite and treated every week with 0.5 mM or 5 mM KNO₃. To evaluate developmental changes in vegetative phase change, plants were analyzed using a stereomicroscope every day after sowing. The day of appearance of cotyledons, day of appearance of first set of leaves, day of appearance of first set of leaves with abaxial trichomes, and the day of bolting were evaluated. Telfer et al. (1997) Development 124(3):645-54.

### Example 2: BT2 is a Central Hub in Plant NUE Regulation

In order to identify candidate genes relevant for the control of NUE, we used the DLS algorithm. Puelma et al. (2012), supra. DLS is a supervised machine-learning algorithm that uses available transcriptome and Gene Ontology (GO) data to infer functional gene networks. DLS has been shown to outperform coexpression gene networks, and is able to generate functional gene networks that integrate multiple biological processes by training on custom-made positive gene sets. The DLS output is a gene network that can be analyzed using standard network topology statistics and tools to pinpoint key genes for the regulation of the biological function of interest. Azuaje (2014) Biology Direct 9(1):12.

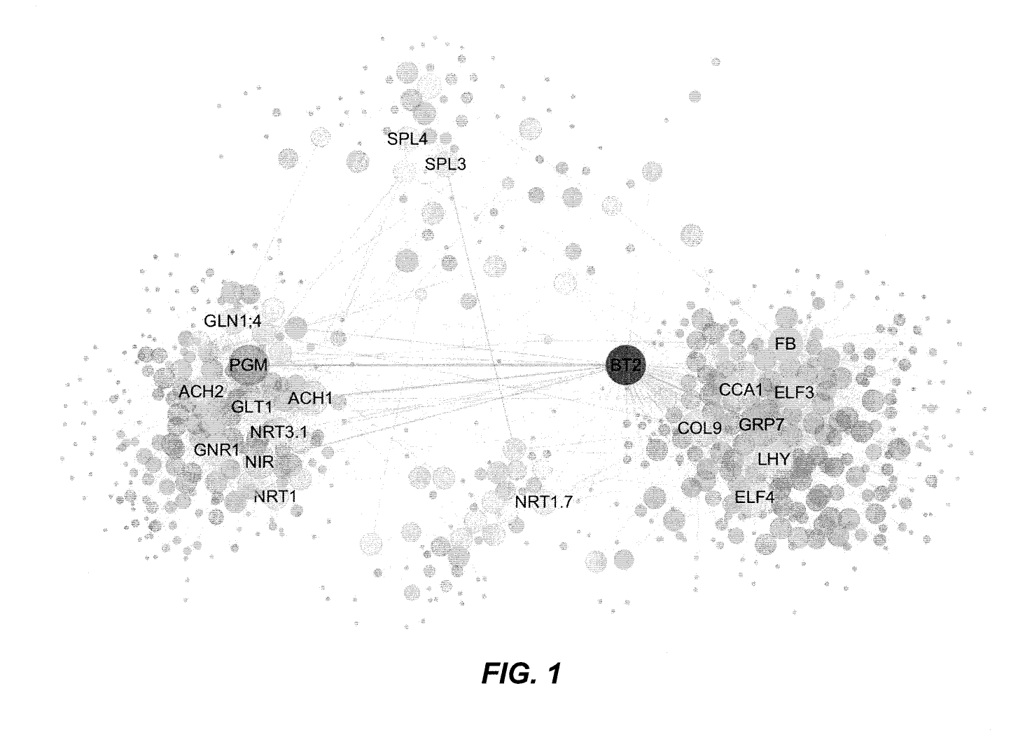

Given that NUE is a complex process that integrates various biological processes, we defined a positive gene set using different biological processes that are known to impact or control plant NUE: nitrate assimilation (GO:0042128), nitrate transport (GO:0015706), ammonium transport (GO:0015696), ammonium response (GO:0060359), nitrogen response (GO:0019740), nitrate response (GO:0010167), regulation of seed development (GO:0080050), organ senescence (GO:0010260), endosperm development (GO:0009960), vegetative to reproductive phase transition of meristem (GO:0010228), vegetative phase change (GO:0010050) and seed maturation (GO:0010431). The union of all these GO terms resulted in a list with 220 genes that was used as the positive set for DLS. Puelma et al. (2012), supra. In addition, we used 2017 microarray experiments obtained from the NASCArrays including 3,911 features or experimental conditions. Using the positive set to train, DLS generated a network containing 351 genes (nodes) connected by functional predictions (edges). FIG. 1. We used CYTOSCAPE software to visualize the resulting sub-network, in which genes are depicted as nodes and edges indicate functional interactions predicted by GENIUS. Node size is proportional to the degree of the node and the node color indicates centrality ranging from yellow (low centrality) to red (high centrality). FIG. 1.

The DLS method used in this work was able to pinpoint genes that have been associated to traits related to NUE in plants in previous reports, including NIR, NR, GLT, GLN, ASN, and LBD. McAllister et al. (2012) Plant Biotechnol. J. 10:1-15; Masclaux-Daubresse et al. (2010), supra; Rubin et al. (2009) Plant Cell 21(11):3567-84. Moreover, over-represented biological processes in the network are known NUE determinants such as senescence, response to nitrate, circadian cycle, and seed development. Li et al. (2013) PloS One 8(4):e62036; Masclaux-Daubresse & Chardon (2011) J. Exp. Bot. erq405; Diaz et al. (2008) Plant Physiol. 147(3):1437-49. These results suggest DLS can effectively predict genes involved in NUE.

The gene network clearly highlighted BT2 (At3g48360) as the most important gene for the overall network structure and topology. FIG. 1. BT2 is the node with the highest degree and betweenness centrality, two metrics that are commonly used to define nodes that are important for network structure (T. Moyano et al. (2015) Methods Mol. Biol. 1284:503-26), making it the best candidate for future experimental validation of its role in controlling NUE. BT2 belongs to a family of BTB and TAZ DOMAIN proteins composed of five members (Robert et al. (2009) Plant J. Cell Mol. Biol. 58(1):109-21) with BT1 (At5g63160) being the closest homolog with 80% sequence identity (Du & Poovaiah (2004) Plant Mol. Biol. 54(4):549-69). Previous studies demonstrated BT1 and BT2 have functional redundancy and reciprocal transcriptional control during gametophyte development. Robert et al. (2009), supra. Therefore, both BT1 and BT2 were selected for experimental validation, as described below.

BT1 and BT2 Affect NUE, Depending on External Nitrate Concentration.

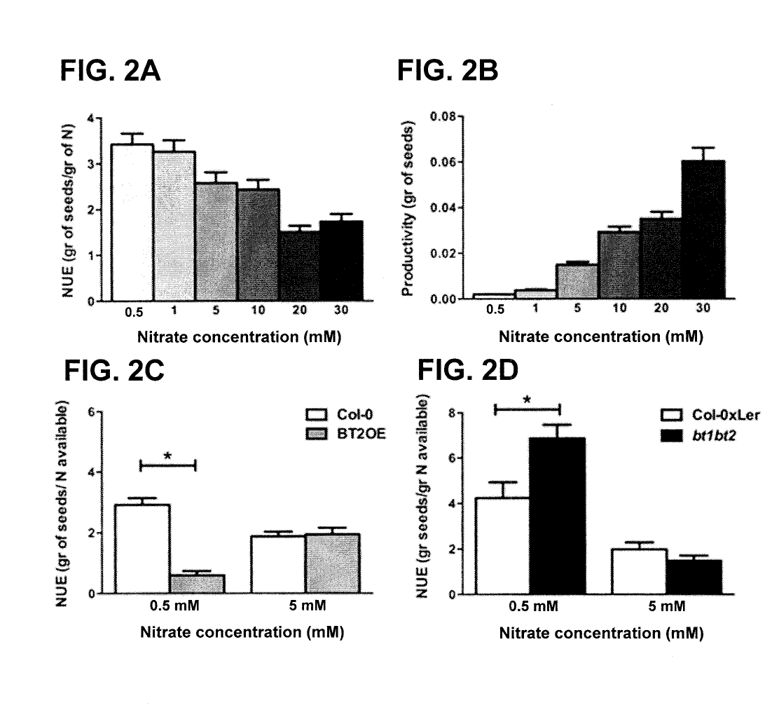

In order to determine the role of BT1 and BT2 in controlling NUE, we measured NUE in wild-type plants, plants overexpressing the BT2 gene (BT2OE) as well as in bt1, bt2, and bt1/bt2 mutant plants under two contrasting nitrate concentrations. FIGS. 2A-2D. NUE was measured by calculating the total seed amount produced per plant (in grams) divided by the total amount of N applied during the entire plant life cycle (in grams). Arabidopsis Col-0 ecotype were grown on an inert substrate and watered once a week with distilled water, and once a week with nutrient solution without N supplemented with 0.5 mM KNO₃, 1 mM KNO₃. 5 mM KNO₃, 10 mM KNO₃, 20 mM KNO₃ or 30 mM KNO₃. All the seeds produced by each plant were collected and weighed.

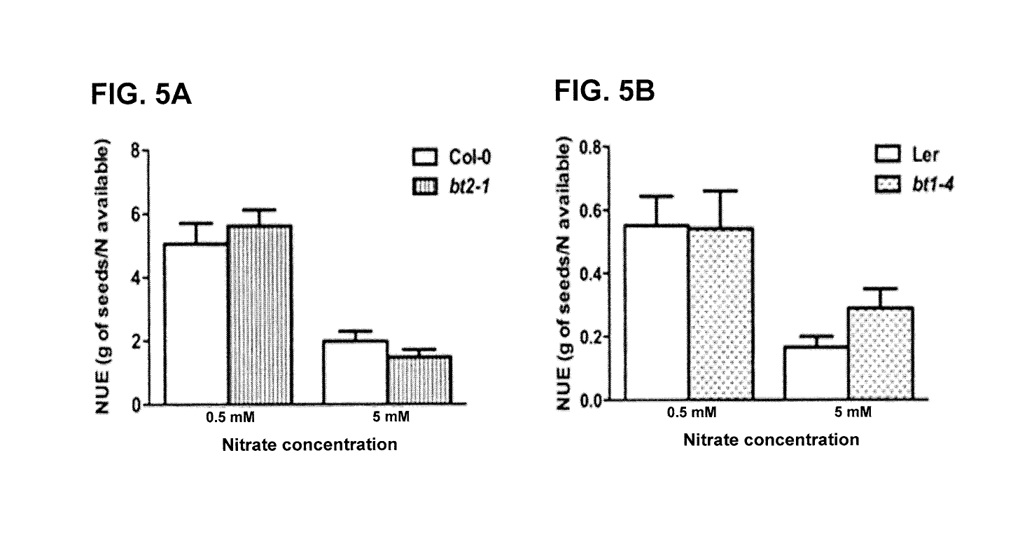

As shown in FIG. 2A, NUE was significantly affected by external nitrate concentration in wild-type plants, with maximum NUE obtained at 0.5 mM and 1 mM nitrate concentrations. NUE decreased as external nitrate concentration increased, with a minimum observed at 20 and 30 mM nitrate. FIG. 2A. This contrasting effect of low or high N availability in plant development has been described previously. Lea & Azevedo (2006) Ann. Appl. Bio. 149(3):243-7; Lemaitre et al. (2008) Plant Cell Physiol. 49(7):1056-65; Ikram et al. (2011) J. Exp. Bot. err244. In contrast to NUE, seed production was significantly (p<0.05) increased by N concentration. FIG. 2B. Overexpression of BT2 decreased NUE as compared to wild-type plants, but only when plants were grown under low nitrate conditions (0.5 mM nitrate). FIG. 2B. bt1 or bt2 single mutants did not show differences as compared to wild-type plants, suggesting functional redundancy. FIGS. 5A and 5B. In contrast, bt1/bt2 double mutant plants exhibited higher NUE as compared to wild-type plants under low nitrate conditions. FIG. 2D. These results indicate BT1/BT2 are negative regulators of NUE under low nitrate conditions.

BT1 and BT2 Affect Juvenile Growth Under Low Nitrate Conditions.

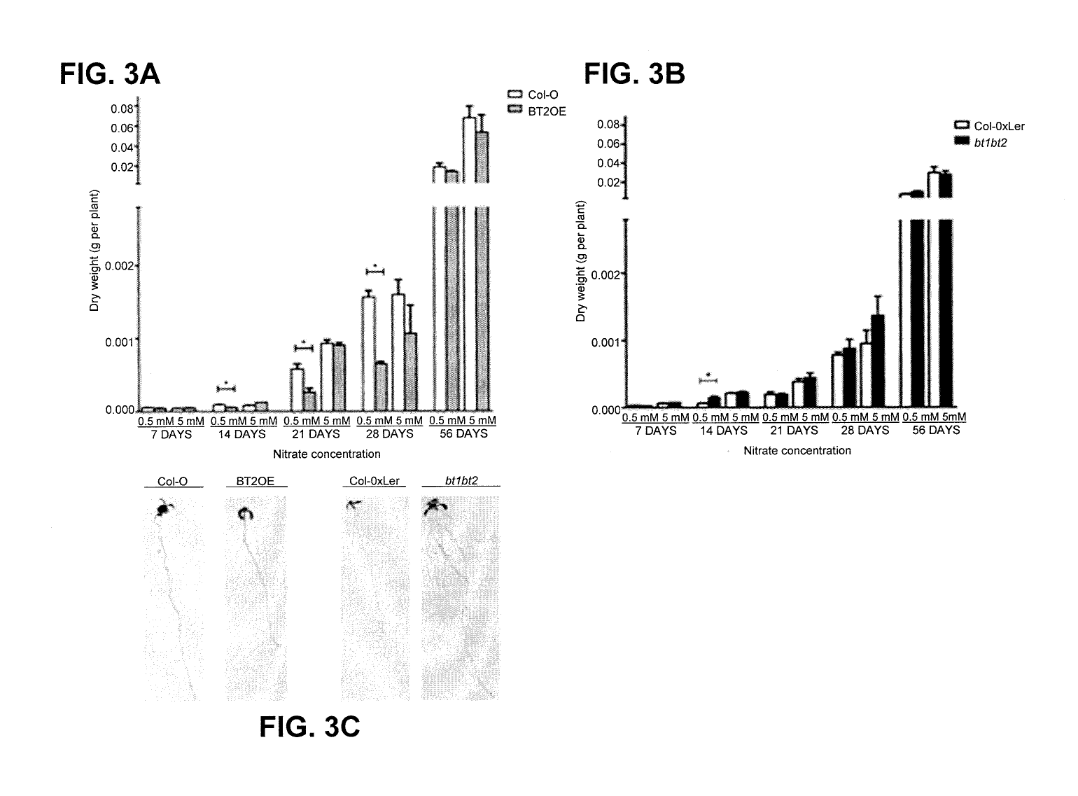

NUE has been shown to change during different developmental phases of plant growth using the Arabidopsis model system. Masclaux-Daubresse & Chardon (2011), supra; Ikram et al. (2011), supra; Poethig (2014) Curr. Top. Dev. Biol. 105:125-52. In order to identify the developmental stages where BT1 or BT2 might have a more prominent impact over NUE, we monitored biomass in wild-type, BT2OE, and bt1/bt2 mutant plants on a weekly basis during their entire life cycle. FIGS. 3A-3C.

BT2OE plants were found to have lower biomass as compared to wild-type plants during the 2^{nd}, 3^{rd}, and 4^{th} weeks after germination in the limiting condition. FIG. 3A. However, bt1/bt2 double mutant plants exhibited higher biomass as compared to wild-type plants only during the 2^{nd} week after germination (FIG. 3B), and only when plants were grown under low nitrate concentrations (FIG. 3C).

Because of the observed timing of the phenotypes, an investigation into whether changes in BT1 or BT2 gene expression levels impacted developmental traits or transitions that occur during this period was conducted. The day of cotyledon emergence and day of appearance of the first set of true leaves as markers of early seedling development, the leaf number of the first leaf with abaxial trichomes, a morphological trait commonly used as markers of the juvenile to adult transition (Telfer et al. (1997), supra), and the day of bolting as marker of reproductive phase transition (Wilkinson & Haughn (1995) Plant Cell 7(9):1485-99; Hempel & Feldman (1994) Planta 192(2):276-86) were measured.

Arabidopsis Col-0 and BT2OE or Col-0×Ler and bt1bt2 were grown on an inert substrate and watered once a week with distilled water and once a week with a nutrient solution without N supplemented with 0.5 mM KNO₃ or 5 mM KNO₃. Every day post-sowing, plants were observed in a stereomicroscope and the day of appearance of true leaves, the number of the first rosette leave with abaxial trichomes, and the day of bolting was recorded.

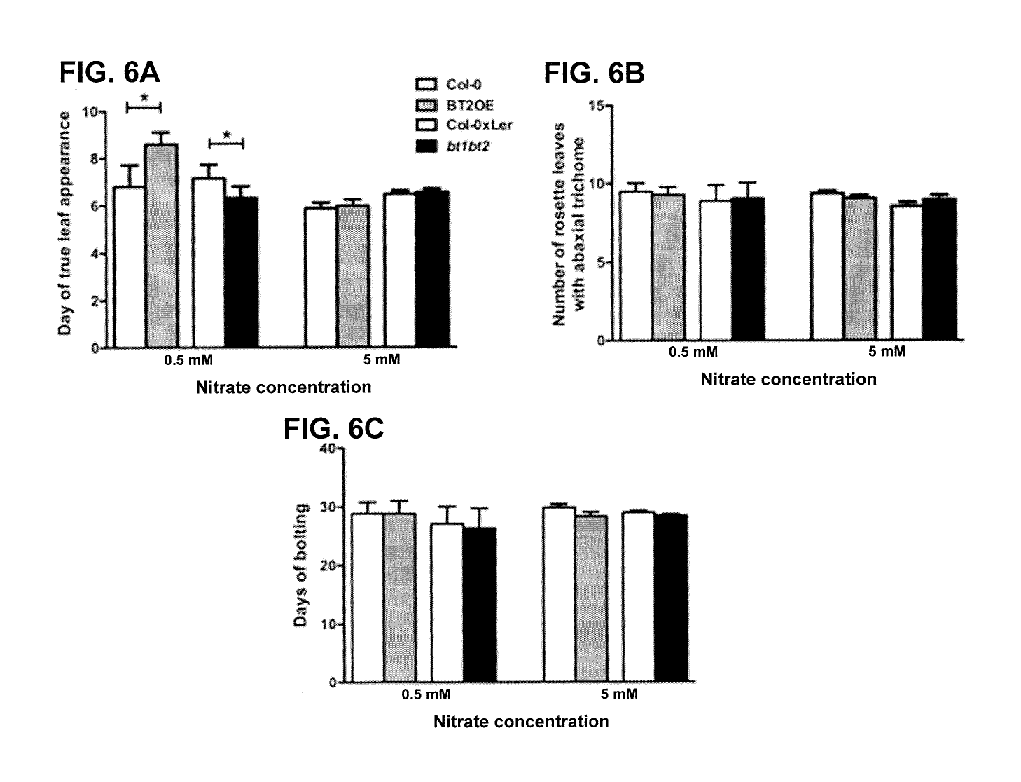

True leaves were found to be visible later in BT2OE and earlier in the bt1bt2 mutant as compared to wild-type plants under low nitrate conditions. FIGS. 6A-6C. No other developmental trait or transition was affected under the experimental conditions evaluated. FIGS. 6A-6C. This indicates BT1 and BT2 have a role in the juvenile stage of plant development that can impact NUE.

BT1/BT2 Repress High Affinity Nitrate Transporters NRT2.1 and NRT2.4 and Nitrate Uptake.

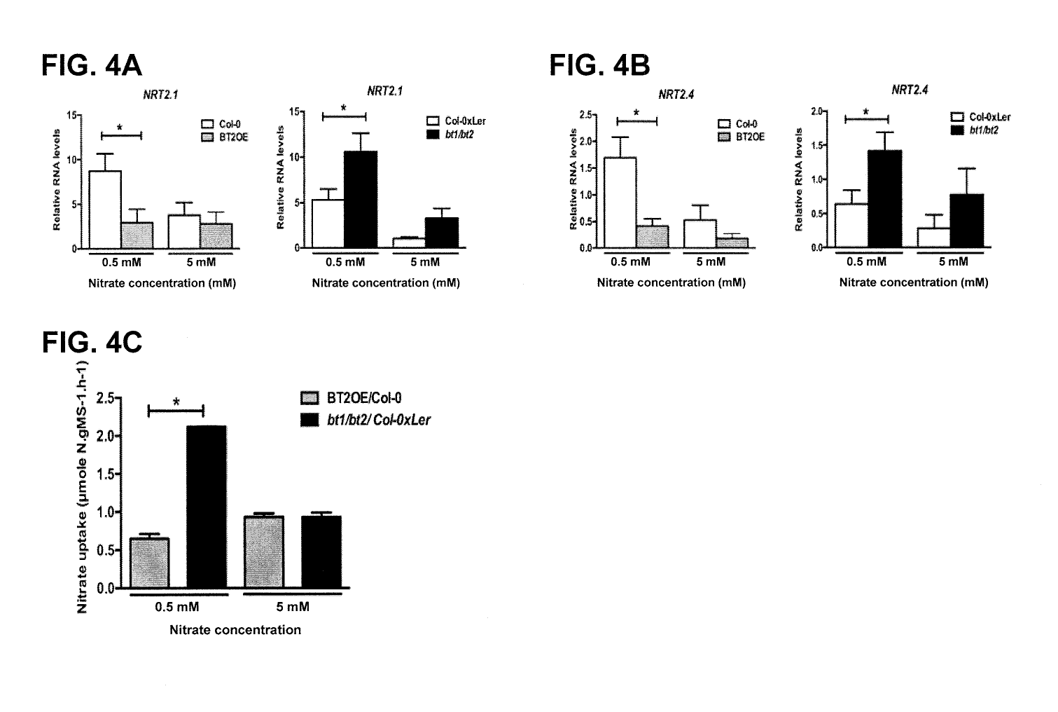

Evaluation of the expression of the NRT2.1, NRT2.2, NRT2.3, NRT2.4, NRT2.5, NRT2.6 and NRT2.7 genes under low and high nitrate conditions in wild-type, BT2OE and bt1/bt2 double mutant plants during the juvenile vegetative phase was conducted. Arabidopsis Col-0 and BT2OE or Col-0×Ler and bt1/bt2 plants were grown for two weeks in agar plates of medium without N supplied with 0.5 mM or 5 mM of KNO₃. NRT2.1 and NRT2.4 transcript levels were analyzed by real-time qPCR in seedlings. FIGS. 4A, 4B. Col-0 and BT2OE and Col-0×Ler and bt1bt2 plants were grown for 28 days, and were treated with a nutrient solution without N supplied with 0.5 mM or 5 mM containing KNO₃ with 10% ¹⁵NO₃ enrichment (w/w). FIG. 4C.

NRT2.1 and NRT2.4 were found to be differentially expressed in BT2OE and the bt1/bt2 mutant as compared with WT plants, specifically under low nitrate concentrations. FIGS. 4A and 4B. NRT2.1 and NRT2.4 have been described as the main high-affinity nitrate transporters involved in nitrate acquisition. Kiba et al. (2012), supra. NRT2.1 and NRT2.4 gene expression levels were significantly lower in BT2OE as compared to wild-type plants. In contrast, NRT2.1 and NRT2.4 gene expression was significantly higher than wild-type plants. FIGS. 4A and 4B. Moreover, nitrate uptake was found to be affected in an opposite manner in the BT2 over-expressing and bt1/bt2 double-mutant plants, consistent with the expression of NRT2.1 and NRT2.4 genes in these plants under low nitrate concentration. FIG. 4B.

These results are consistent with the growth phenotype found for these plants being due, at least in part, to misregulation of nitrate transport by NRT2.1 and NRT2.4 under low nitrate concentrations. bt1/bt2 double mutant plants were considerably bigger than wild-type plants under low nitrate conditions. FIG. 3C. These results indicate that BT1 and BT2 control plant growth by controlling the expression of the NRT2.1 and NRT2.4, and thus nitrate transport under low nitrate concentrations.

Our results indicate BT1/BT2 function under low nitrate conditions to impact NUE. One of the main factors that can affect NUE in plants is the control of N uptake. Masclaux-Daubresse et al. (2010), supra. Nitrate transporters from the NRT2 family are the main transporters involved in Arabidopsis nitrate transport under low nitrate concentrations. Tsay et al. (1993) Cell 72(5):705-13; Huang et al. (1999) Plant Cell 11(8):1381-92; Filleur et al. (2001) FEBS Lett. 489(2-3):220-4; Kiba et al. (2012) Plant Cell 24(1):245-58.

Previous results suggested NUE was independent of N supply, and only dependent on plant genotype. Chardon et al. (2010) J. Exp. Bot. 61(9):2293-302. However, NUE was found to decrease as N supply increased under the experimental conditions. This apparent discrepancy is due to the way NUE was measured by the authors of Chardon et al. (2010), supra. In this work, NUE was measured as the ratio of rosette biomass to N concentration in the rosette, without normalizing by N supply. Different metrics exist to determine NUE, depending on the specific crop and trait studied. Since one of our goals was to find genes involved in NUE that might be used as targets for improving this trait in different cultivars, we chose to use the grain yield normalized per unit of N available, a common measure of NUE in crops (Good et al. (2004) Trends Plant Sci. 9(12):597-605), which is broadly applicable to different plant species and economically important plants such as cereals.

Mechanisms that impact traits that are conditioned by the environment are important targets for crop productivity. Gifford et al. (2013) PLoS Genetics 9(9):e1003760. Increased growth under low N in bt mutant plants reaches values comparable to plants grown under sufficient N conditions. Our results show NUE is increased by nearly 20% in mutant Osbt plants. It is estimated that a 1% increase in NUE of crops could save US$1.1 billion annually. Kant et al. (2010), supra. Our results offer a prime target for new biotechnologies to improve crop production in economically and environmentally sustainable manners.

1. A method for producing a transgenic plant cell, the method comprising:
introducing at least one heterologous polynucleotide into the plant cell, wherein the heterologous polynucleotide hybridizes under stringent conditions to a polynucleotide selected from the group consisting of SEQ ID NO:1, SEQ ID NO:3, SEQ ID NO:7, SEQ ID NO:9, SEQ ID NO:11, SEQ ID NO:13, SEQ ID NO:15, and the complements thereof.

2. The method according to claim 1, wherein the polynucleotide is selected from the group consisting of:
a polynucleotide encoding a mutant Bric-a-Brac/Tramtrack/Broad-1 (BTB1) or Bric-a-Brac/Tramtrack/Broad-2 (BTB2) polypeptide; and
a polynucleotide encoding an antisense ribonucleic acid molecule targeting a BT1 or BT2 gene,
thereby producing a transgenic plant cell.

3. The method according to claim 2, wherein the method comprises introducing:
a polynucleotide encoding a mutant BTB1 polypeptide and a polynucleotide encoding a mutant BTB2 polypeptide;
a polynucleotide encoding an antisense ribonucleic acid molecule targeting a BT1 gene and a polynucleotide encoding an antisense ribonucleic acid molecule targeting a BT2 gene;
a polynucleotide encoding a mutant BTB1 polypeptide and a polynucleotide encoding an antisense ribonucleic acid molecule targeting a BT2 gene; or
a polynucleotide encoding a mutant BTB2 polypeptide and a polynucleotide encoding an antisense ribonucleic acid molecule targeting a BT1 gene.

4. The method according to claim 2, wherein the heterologous polynucleotide encodes a mutant BTB1 polypeptide derived from a BTB1 polypeptide selected from the group consisting of SEQ ID NO:2, SEQ ID NO:10, SEQ ID NO:12, SEQ ID NO:14, and SEQ ID NO:16.

5. The method according to claim 4, wherein the heterologous polynucleotide is a bt1 polynucleotide selected from the group consisting of a deletion mutant, a polynucleotide encoding a truncated BTB1 polypeptide, a polynucleotide encoding a polypeptide having at least 80% but less than 100% sequence identity with a BTB1 polypeptide selected from the group consisting of SEQ ID NO:2, SEQ ID NO:4, SEQ ID NO:10, SEQ ID NO:12, SEQ ID NO:14, and SEQ ID NO:16, and a polynucleotide comprising a non-functional regulatory element.

6. The method according to claim 5, wherein the bt1 polynucleotide encodes the polypeptide of SEQ ID NO:8.

7. The method according to claim 6, wherein the bt1 polynucleotide is SEQ ID NO:7.

8. The method according to claim 2, wherein the heterologous polynucleotide is a polynucleotide selected from the group consisting of a deletion mutant, a polynucleotide encoding a truncated BTB2 polypeptide, a polynucleotide encoding a polypeptide having at least 80% but less than 100% sequence identity with SEQ ID NO:2 or SEQ ID NO:4, and a polynucleotide comprising a non-functional regulatory element.

9. The method according to claim 8, wherein the heterologous polynucleotide encodes a mutant BTB2 polypeptide derived from the BTB2 polypeptide of SEQ ID NO:4.

10. The method according to claim 9, wherein the bt2 polynucleotide encodes the polypeptide of SEQ ID NO:10.

11. The method according to claim 10, wherein the bt2 polynucleotide is SEQ ID NO:9.

12. The method according to claim 2, wherein the heterologous polynucleotide encodes an antisense ribonucleic acid molecule targeting a BT1 gene, wherein the antisense ribonucleic acid molecule is at least 18 nucleotides in length, and wherein the antisense ribonucleic acid molecule hybridizes to a polynucleotide comprising SEQ ID NO:1 under highly stringent conditions.

13. The method according to claim 12, wherein the heterologous polynucleotide is at least 95% identical to at least 18 contiguous nucleic acids of SEQ ID NO:5.

14. The method according to claim 2, wherein the heterologous polynucleotide encodes an antisense ribonucleic acid molecule targeting a BT2 gene, wherein the antisense ribonucleic acid molecule is at least 18 nucleotides in length, and wherein the antisense ribonucleic acid molecule hybridizes to a polynucleotide comprising SEQ ID NO:3 under highly stringent conditions.

15. The method according to claim 14, wherein the heterologous polynucleotide is at least 95% identical to at least 18 contiguous nucleic acids of SEQ ID NO:6.

16. The method according to claim 1, wherein the heterologous polynucleotide is optimized for expression in a plant cell.

17. The method according to claim 1, wherein the heterologous polynucleotide is at least 80% identical to one or more of SEQ ID NO:1, SEQ ID NO:3, SEQ ID NO:7, and SEQ ID NO:9.

18. The method according to claim 1, wherein the heterologous polynucleotide encodes a polypeptide that is at least 90% identical to one or more of SEQ ID NO:2, SEQ ID NO:4, SEQ ID NO:8, and SEQ ID NO:10.

19. The method according to claim 1, wherein the heterologous polynucleotide is operably linked to a plant promoter.

20. The method according to claim 19, wherein the plant promoter is a constitutive promoter, tissue-preferred promoter, tissue-specific promoter, or inducible promoter.

21. The method according to claim 1, wherein introducing the heterologous polynucleotide into the plant cell comprises transforming the plant cell with a nucleic acid molecule or introgressing the heterologous polynucleotide into a plant comprising the plant cell.

22. A transgenic plant cell produced by the method according to claim 1, wherein the plant cell comprises the heterologous polynucleotide.

23. A transgenic plant material comprising the plant cell of claim 22.

24. A transgenic plant comprising the plant cell of claim 22, wherein the plant comprises increased nitrogen use efficiency, as compared to a plant of the same variety that does not comprise the heterologous polynucleotide.

25. The transgenic plant of claim 24, wherein the increased nitrogen use efficiency comprises increased growth of the transgenic plant under limited nitrogen conditions, as compared to the growth of a plant of the same variety that does not comprise the heterologous polynucleotide in the same limited nitrogen conditions.

26. A method for increasing nitrogen use efficiency in a plant, the method comprising:
introducing into the plant at least one at least one means for silencing bt1 expression in a plant, and/or at least one means for silencing bt2 expression in a plant.

27. The method according to claim 26, wherein the method comprises introducing into the plant a means for silencing bt1 expression in a plant, and a means for silencing bt2 expression in a plant.

28. The method according to claim 27, wherein the means for silencing bt1 expression in a plant is the polynucleotide of SEQ ID NO:5 or the polynucleotide of SEQ ID NO:7, and the means for silencing bt2 expression in a plant is the polynucleotide of SEQ ID NO:6 or the polynucleotide of SEQ ID NO:9.

29. The transgenic plant cell of claim 22, wherein the plant cell is not regenerated into a plant.

30. A composition produced from the plant of claim 24, wherein the composition comprises the heterologous polynucleotide, and wherein the composition comprises a plant part, plant fiber, plant protein, plant meal, and/or plant oil.


SEQUENCE LISTING (16 sequences)

SEQ ID NO: 1 - DNA, 2223 bp, Arabidopsis thaliana
ccatcatcaaattaaaaccctccagcctccaacaacaaaatttaccaacactctctctttctctttctctctgaaaacttaaaacgatggctataaccgccactcaaaacgacggcgtttcactaaacgccaataagatttcgtatgatcttgtggaaactgatgttgagatcatcacctccggtcgtcgtagtattccggcacactccggcatcctcgtaagtttcttttcttcttactttctttgtatgatcgaatatttttttattttggtgcttatcttttttgtttgtttaaaattgttaaatgcaggcttcggtctcaccggtactgacgaacatcatcgagaagccgaggaagattcacggcggatcatcgaagaaagttattaagattctcggtgttccatgcgacgccgtttcagtcttcgtcagattcctctattctccgaggttcgtctttctctctttttcatatcttcttgatcgtcagatttttttttttaatccgtagatttattacttcaaaaatacaaatcatactaaaaagaatgcttgattaaatacatcatcttaaagatctttaccaaactcgacttttccaacgttttaatcatacattaaaatcactacaaccagattgcgattgatttagacttgtttcatattcaaaaacgtgtgaaacacaacacaaggaacacgaaactagtaccaacatcaaatttgaataaatcaacggcaaattttacccttagtatgcaaaatgactagaatacccctatatgtatcttttgtttagtgtcacggagaatgagatggaaaaatacggaatccatctactggctttatcacacgtgtatatggtgactcagttaaagcaacggtgcactaaaggcgtcggtgagagagtaaccgccgaaaacgttgtcgatattctccagctggctcgtctctgcgacgcacctgacctctgtctcaagtgtatgcgattcattcactacaagttcaagaccgttgaacagacagaaggatggaagtttcttcaagaacacgatccttttcttgaacttgacattctccaattcatcgacgatgcagaatcggtaattaactcaaaccacacttttttgttttctttttttttggttcttcaagaaaactctgttttattgaattaggtttcttttgggtcaatttcttgcagaggaagaaaagaagaaggagacacagacgagaacagaatctgtatttgcagctgagtgaagccatggaatgtatagaacacatatgcaccgaaggttgcacactggtcggaccatcgtctaacttagacaacaagtcaacatgtcaagcaaaacccggtccatgcagtgcgttttcgacttgttacggacttcaacttcttatacgtcactttgcagtatgcaagaaaagagtcgatggcaaaggttgtgtccgatgcaagagaatgattcaactccttagactccattcttcgatttgtgaccaatctgaatcttgccgtgtccctctttgcaggtaaaaatccgacattttagaataatcaaaaccacattttaaatgtttctaatgataaaagtttttcacttttcgatagtagatctcatttgatttttttttaactttcggcgccaataataattatctttttgtggtggtagcctacttattagatgatgagtcgtgatcggacggctggaaatattttgtttggtagaattattaaaagatattttaagaaatgagaatctgatctcgttgtcaactttttatttgttttggtgattggagtaggcaatataagaatagaggtgaaaaggacaagaaaatggttgaggacacgaagtggaaggttctggtgagaagagtagcgtctgctaaagccatgtcttcgttgtctcaatcaaagaagaaaaaaagtgaagtgttatttaaagaagaagcagaagatttgatcagaatccggaacaagttaatgtgaatatacaatatatgtgtttttattagattatatagtgagcgattgttagaagccattaattacttgagtcggaaggtttatttgtttgttatttgtctctcggactggttgattgggttaaacttgtcaatgagtaaccctttgaatctgtcgttttgtttggtcggtcgaattatatagaaatgtattattggttataaaatggtattagggtttttggag

SEQ ID NO: 2 - protein, 365 aa, Arabidopsis thaliana
MAITATQNDGVSLNANKISYDLVETDVEIITSGRRSIPAHSGILASVSPVLTNIIEKPRKIHGGSSKKVIKILGVPCDAVSVFVRFLYSPSVTENEMEKYGIHLLALSHVYMVTQLKQRCTKGVGERVTAENVVDILQLARLCDAPDLCLKCMRFIHYKFKTVEQTEGWKFLQEHDPFLELDILQFIDDAESRKKRRRRHRREQNLYLQLSEAMECIEHICTEGCTLVGPSSNLDNKSTCQAKPGPCSAFSTCYGLQLLIRHFAVCKKRVDGKGCVRCKRMIQLLRLHSSICDQSESCRVPLCRQYKNRGEKDKKMVEDTKWKVLVRRVASAKAMSSLSQSKKKKSEVLFKEEAEDLIRIRNKLM

SEQ ID NO: 3 - DNA, 2334 bp, Arabidopsis thaliana
caaactaccaaacctacagaaatctctcttcttcttcttctctacaaaatttcaatggaagctgttcttgtcgcaatgtccgtccccgccacaacggaagacgacggattttcgttaatcaccgataaactttcatataatctaacgccgacgtcggacgttgagatcgttacctccgataaccgtcggattccggcacactccggcgttctggtaagttgttatttattttggtttacggtttacatttttctggtttggtttttagctaaaatttgtggataatgcaggcttcagcttcaccggtactgatgaacatcatgaagaaaccgatgagacgttacagaggctgtggatctaaaagagtcatcaagattcttggcgttccatgcgacgccgtttcggtttttatcaaattcctctactcttctaggttggtttggttcggttattcttcttgaattttccaaatgtagaaacttcaaaccgatggttatttccggtactacacaattttcccggtttttctttttcatacacggatttgaagaaactctgtttttttttgggctattttcttggggaaacaatagctgtcaggaaatgactaaactacccttttgcgtctctgttttagtttgacggaggatgagatggagagatatggaatccatcttttagcgttgtctcacgtctacatggtcacccagctgaagcaacggtgctcaaaaggcgttgtacaacgtttaactacagagaacgtagtcgacgtcctccagctagctcggctctgcgacgcacccgatgtttgccttcgatctatgcgtctgatccattcgcagtttaagaccgttgagcagactgaaggatggaagtttattcaagaacatgatcctttcctcgagctcgacattctccagttcatcgatgacgccgaatcggtaaacttacttatatgttttgaaaacacagcatctctgtttttcttagtaccttcgataacacattttgttcagctaggtttagtttttgttccgcgctagtaaaaagataatttttttttttttaaattcagtttatttagtttggcatgttgcacaaagtttagagtattagttaactcatgtttgattagatttggtttatatacctaattaaccccaaaacatttggtcatttttctgattatgtgttgatttataatttaacgcagaggaagaaaagaaggcgacgacatagaaaggagcaggatctttacatgcaactaagcgaggcaatggagtgcatagagcacatatgcacacaaggttgtacattggtcggtccatcaaacgtagtagataacaacaaaaagtcaatgactgcggaaaagtcagagccttgtaaggcgttttccacgtgttatggtcttcagcttttgatacgtcactttgccgtatgcaagaggaggaataacgataagggttgtcttcgttgcaagcggatgcttcaactctttagactccattctttgatttgtgatcaacccgattcttgtcgcgtccctctctgcaggtatgtcccaacttgttttgttaggaatttcacatttgaaataaatcaaaaagtatgagcgaatccatttacgcttaattagtaagtttgaaattgatgatttttagtatgtttatgaataaaaaccaatgttttgttgaaagtgaaaaaatttcatttttggtaagagacaaggtttgtttatctttttgtgtagacccacctttagaaaagttgcaaattgctatgtgaattgatgatgggtcgtaatccgacggctgcaaattgttgccatttcttgaaatttgtagatattttaatagaatctcatgtgttcttttcaactcttttttttgggactaggcaatttaggaaacggggagaacaagacaagaaaatgggtgaagacaccaagtggaagcttctggtgacaagggttgtgtctgcaaaagctatgacgtcgttgtgtcagtcaaagaagaacaaatgtgaacaagcacaaggggtttaatcaaaaccaggaaggagttagttaggtttgaagaagattaagtaagaagctttcttataatttgatatccattctttttgtagtataatagtatttgttagttcgattctttattagaggggtttagtttgtataggagtaaacccttttaaattggctgtataagtaggtcatattcaattgttttgttctatttatctgaaaaatgtcagcatgtaatacaaaacaaataaatgttcaaattaaagggaggattacaagaggtgtttttaatgttattgactttggcc

SEQ ID NO: 4 - protein, 364 aa, Arabidopsis thaliana
MEAVLVAMSVPATTEDDGFSLITDKLSYNLTPTSDVEIVTSDNRRIPAHSGVLASASPVLMNIMKKPMRRYRGCGSKRVIKILGVPCDAVSVFIKFLYSSSLTEDEMERYGIHLLALSHVYMVTQLKQRCSKGVVQRLTTENVVDVLQLARLCDAPDVCLRSMRLIHSQFKTVEQTEGWKFIQEHDPFLELDILQFIDDAESRKKRRRRHRKEQDLYMQLSEAMECIEHICTQGCTLVGPSNVVDNNKKSMTAEKSEPCKAFSTCYGLQLLIRHFAVCKRRNNDKGCLRCKRMLQLFRLHSLICDQPDSCRVPLCRQFRKRGEQDKKMGEDTKWKLLVTRVVSAKAMTSLCQSKKNKCEQAQGV

SEQ ID NO: 5 - DNA, 2223 bp, Artificial Sequence; identical to SEQ ID NO: 1
  Reverse complement of A. thaliana bt1

SEQ ID NO: 6 - DNA, 2334 bp, Artificial Sequence; identical to SEQ ID NO: 3
  Reverse complement of A. thaliana bt2

SEQ ID NO: 7 - DNA, 2446 bp, Artificial Sequence
  Mutant bt1 polynucleotide
ccatcatcaaattaaaaccctccagcctccaacaacaaaatttaccaacactctctctttctctttctctctgaaaacttaaaacgatggctataaccgccactcaaaacgacggcgtttcactaaacgccaataagatttcgtatgatcttgtggaaactgatgttgagatcatcacctccggtcgtcgtagtattccggcacactccggcatcctcgtaagtttcttttcttcttactttctttgtatgatcgaatatttttttattttggtgcttatcttttttgtttgtttaaaattgttaaatgcaggcttcggtctcaccggtactgacgaacatcatcgagaagccgaggaagattcacggcggatcatcgaagaaagttattaagattctcggtgttccatgcgacgccgtttcagtcttcgtcagattcctctattctccgaggttcgtctttctctctttttcatatcttcttgatcgtcagatttttttttttaatccgtagatttattacttcaaaaatacaaatcatactaaaaagaatgcttgattaaatacatcatcttaaagatctttaccaaactcgacttttccaacgttttaatcatacattaaaatcactacaaccagattgcgattgatttagacttgtttcatattcaaaaacgtgtgaaacacaacacaaggaacacgaaactagtaccaacatcaaatttgaataaatcaacggcaaattttacccttagtatgcaaaatgactagaatacccctatatgtatcttttgtttagtgtcacggagaatgagatggaaaaatacggaatccatctactggctttatcacacgtgtatatggtgactcagttaaagcaacggtgcactaaaggcgtcggtgagagagtaaccgcctttaccgaccgttaccgaccgttttcatccctacttgtaactgaaataaaaacagagttttcatagacttcagaactaaatcaaccaaaaccaaataagctgaaaaataaaataaaaaacgaatgcatagcaaaaactggtattaagctttgaattctaaactataagacaagttttgtagtttaccgcgggattgtccaagcgatcaagctcgtggccatcggggatactcccagagatatactgccagccaaggatgtcacattccaaatcagcatccaaaagtgtatcccaaaaatacttcattccccatttccatggaaggagaaaaaacttcacagcaaagcttgaaacaatcactcttattctgttatgcatccatccggtagcccaaagctctctcattccggcatccaccaacggataaccggtcctgccttgtctccaggccttgaacttatcaacatcagcatcccaagggaaaaaccgaaaatgactcaacaacgattgctcgtgagtaaacgggaagttgaaacatatataccgagaatactctcttaaaccgattcccctaagaaaaagatctgcactttcttctccttcactgttccactggtcggaccatcgtctaacttagacaacaagtcaacatgtcaagcaaaacccggtccatgcagtgcgttttcgacttgttacggacttcaacttcttatacgtcactttgcagtatgcaagaaaagagtcgatggcaaaggttgtgtccgatgcaagagaatgattcaactccttagactccattcttcgatttgtgaccaatctgaatcttgccgtgtccctctttgcaggtaaaaatccgacattttagaataatcaaaaccacattttaaatgtttctaatgataaaagtttttcacttttcgatagtagatctcatttgatttttttttaactttcggcgccaataataattatctttttgtggtggtagcctacttattagatgatgagtcgtgatcggacggctggaaatattttgtttggtagaattattaaaagatattttaagaaatgagaatctgatctcgttgtcaactttttatttgttttggtgattggagtaggcaatataagaatagaggtgaaaaggacaagaaaatggttgaggacacgaagtggaaggttctggtgagaagagtagcgtctgctaaagccatgtcttcgttgtctcaatcaaagaagaaaaaaagtgaagtgttatttaaagaagaagcagaagatttgatcagaatccggaacaagttaatgtgaatatacaatatatgtgtttttattagattatatagtgagcgattgttagaagccattaattacttgagtcggaaggtttatttgtttgttatttgtctctcggactggttgattgggttaaacttgtcaatgagtaaccctttgaatctgtcgttttgtttggtcggtcgaattatatagaaatgtattattggttataaaatggtattagggtttttggag

SEQ ID NO: 8 - protein, 35 aa, Artificial Sequence
  Mutant BT1 polypeptide
MAITATQNDGVSLNANKISYDLVETDVEIITSGRR

SEQ ID NO: 9 - DNA, 6414 bp, Artificial Sequence
  Mutant bt2 polynucleotide
caaactaccaaacctacagaaatctctcttcttcttcttctctacaaaatttcaatggaagctgttcttgtcgcaatgtccgtccccgccacaacggaagacgacggattttcgttaatcaccgataaactttcatataatctaacgccgacgtcggacgttgagatcgttacctccgataaccgtcggattccggcacactccggcgttctggtaagttgttatttattttggtttacggtttacatttttctggtttggtttttagctaaaatttgtggataatgcaggcttcagcttcaccggtactgatgaacatcatgaagaaaccgatgagacgttacagaggctgtggatctaaaagagtcatcaagattcttggcgttccatgcgacgccgtttcggtttttatcaaattcctctactcttctaggttggtttggttcggttattcttcttgaattttccaaatgtagaaacttcaaaccgatggttatttccggtactacacaattttcccggtttttctttttcatacacggatttgaagaaactctgtttttttttgggctattttcttggggaaacaatagctgtcaggaaatgactaaactacccttttgcgtctctgttttagtttgacggaggatgagatggagagatatggaatccatcttttagcgttgtctcacgtctacatggtcacccagctgaagcaacggtgctcaaaaggcgttgtacaacgtttaactacagagaacgtagtcgacgtcctccagctagctcggctctgcgacgcacccgatgtttgccttcgatctatgcgtctgatccattcgcagtttaagaccgttgagcagactgaaggatggaagtttattcaagaacatgatcctttcctcgagctcgacattctccagttcatcgatgacgccgaatcggtaaacttacttatatgttttgaaaacacagcatctctgtttttcttagtaccttcgataacacattttgttcagctaggtttagtttttgttccgcgctagtaaaaagataatttttttttttttaaattcagtttatttagtttggcatgttgcacaaagtttagagtattagttaactcatgtttgattagatttggtttatatacctaattaaccccaaaacatttggtcatttttctgattatgtgttgatttataatttaacgcagaggaagaaaagaaggcgacgacatagaaaggagcaggatctttacatgcaactaagcgaggcaatggagtgcatagagcacatatgcacacaaggttgtacattggtcggtccatcaaacgtagtagataacaacaaaaagtcaatgactgcggaaaagtcagagccttgtaaggcgttttccacgtgttatggtcttcagcttttgatacctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcggtacccggggatcctctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggctaatctggggacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacaggtcttgaaatttgtagatattttaatagaatctcatgtgttcttttcaactcttttttttgggactaggcaatttaggaaacggggagaacaagacaagaaaatgggtgaagacaccaagtggaagcttctggtgacaagggttgtgtctgcaaaagctatgacgtcgttgtgtcagtcaaagaagaacaaatgtgaacaagcacaaggggtttaatcaaaaccaggaaggagttagttaggtttgaagaagattaagtaagaagctttcttataatttgatatccattctttttgtagtataatagtatttgttagttcgattctttattagaggggtttagtttgtataggagtaaacccttttaaattggctgtataagtaggtcatattcaattgttttgttctatttatctgaaaaatgtcagcatgtaatacaaaacaaataaatgttcaaattaaagggaggattacaagaggtgtttttaatgttattgactttggcc

SEQ ID NO: 10 - protein, 71 aa, Artificial Sequence
  Mutant BT2 polypeptide
MEAVLVAMSVPATTEDDGFSLITDKLSYNLTPTSDVEIVTSDNRRIPAHSGVLVSCYLFWFTVYIFLVWFL

SEQ ID NO: 11 - DNA, 4401 bp, Oryza sativa
ccctcagatgcatagtactcgcacgccgcgacgcctctgctgaacatttcgctctcttccaagatattttttcctcccacgattccttcctcccatccctgtggggattcatcggctcatccgtactagcacaagatgtgcgagggtgtgcgggccgccggcgacgccgccgccgccgccgacgtcgacgtcatcacctcctccggccgccggaggatccccgcgcattccactgttcttgtaagcttctcctctttcatcagcaaattcatgaatccttttagctggagatagtttctgtggtcaaattctggttggcgtcgggcgtgttctgacgaggaaatctccgtttaattctttgtttgtttggtttgatcaccaggcgtcggcgtcgccggtgctggagagcatcctgcagcgccgcctgaagaaggagagggacgccgccgccggcggcggcaaggtccgcagggccgtcgtcctgatccgcggcgtcaccgacgacgccgcggccgccttcgtccgcctcctctacgccggcagcaggtaccgtgccctcgtccattccattcggccattcccctctaggatgggatttctgctggtagtctttgcttttcgagcatccgtttcttgcaaaagatgggcgattagttgaagaattaatggtcttcgaagactaattactcgtattaactcacgagggggaaagaaaaacatgtcacacgtgtaggccacgctgacaagaaactcgatctcttcttctatggtttagttgaaaattatatatttttagtttatttttttattagagggtacgttttttctgttgatcatggtagcagtaattggctactaataattgctacctaaactatgaaaacggtagatacgacgaaatgatgttgaaagtttttaagcgatgataaacagtgggtgaaaatatgcttaatcactattctgtatctgtagcagaacgagcaaaaccttcggagcgaatgctgtaggaaaaaaactgaaaaaaaaaatctggtgtccaaagtccttgctagataggcagtccaatcaaggcaggcccggctaaaccacggatattttcttcatctatcacacagcttacagatccaatgaaccattccaagaatctcgctaagtgattagacagatcgctttggcagcctccgtgataaaatcccctccaagttggccaatttgccgattttttgtgcctcatcaaactgtgcgagtcttgacaaggcaggtcaggtaaggtgacacaacacaaccccacacaaatgacacaaacagagcatcgaaacatcgccatctccagctccgagagccgtaaacaaatctcgtggagcagaatccatgccttgcctcggcgcgtcgacttggacggcacagacagagaaaaaaaaagagagaaaaagtagtagtaaagcgaagcaacgataagtactagaccactagagcagtaggagaagtcgagaagaggatgattttggctttctttttccgccgcttggtttggagtttggtgtggtagcgcctgcctagttggaaccctccccttgcaggcgagcaccaagcaggcagccgatctcgtcggcgccacaagaaaaatcgttgctgtcgctggtgcggcttgcagggccgggaccgctcgccgcctcagccatcatctcgtgttggtttttgttgaaaaaaaaaaagaactctctttttgtgtgtggagatgcagaggtagggacctgcccaaactgtcccagtgtcccgatccgtttgaaacgaatgaagactttcgtgcctttctcaacattagtaatagagtaaatttcacaaaagtagaagttttatggttcaagttgcagaaaatcacacacattttgacacttgacacttgacacttaagtacatatatttcggtagtttagtttcataaaaccacactatcgatgaatggattcacccgtgagatgatatggttgttccacccgtgagatgacgtggttgttccatctaagatgaggacgtgacatcatattaatttcgtgcgatggctggtaatagaatgccatgtcctcgtcttaggtgtaatagccacatcatctcacgggtgaatctatacatcgataatgtagttttgtgaaactacacaaccaaaatatatgtactcaagtgccaagtgtcaaagtgtatgtgattttctgtaacttggaccacaaaacgtgtagtagaaatatttgagaaaaaaacatttgcatattagacgtgctaatactgtgtaaattttgttaactaattgtgcagtggtgatgaggaggagattgacgagaagagcgcggcgcagatgctggtgctggcgcacgcgtaccgggtgccgtggctgaagcggcggtgcgagggggcgatcgggtcgcggctcacggcggagtcggtggtggacacgatgcagctggcggcgctgtgcgacgcgccgcagctgcacctgcgctgcaccaggctgctcgccaaggagttcaaggccgtggagaagaccgaggcgtggcgattcctccaggagaacgacccctggctcgagctcgacatcctccagcgcctccacgacgccgacctggtatacatcattatcgctgttcatcaacatggtagaattttacaggaatttcacacaaaaaaaattcaaactttgaattgtgcactcgatgtggtgtggggtatccatgttgggcgcttttgtaaatggctctgtgacacacggttccattgacaccgaaatgcatgggaggatttgcacagtgggtcgtgcactaggggaacatgattttgagcgtgttttactgggatctttcgagccttaagtccctggaatagccgggggccaccgtgtcgcactcgtgcacacattttatgtcgagacgtgctttgcgttttgtggttggttggtggattgaatgagaaatttcttgtatctgggggaggctaggtcggtccggttggtctgtccaaccacgttgaatggacttcgaacggcttaccgtctcgaagtttcccgatcctaaacgtttttgttatggtcgaatgattttgattttgacaagaacctaattaggacctgtttactttgatgtcattttcaatcttaccaaattttggtaaagttgtcaaaattgtggctacatttagtttgctgtcaaattttgataactatataagaaatcctgccaattaccaaaattttgacaactatgccaaaattttggtaatgtttttttcttttgcattaaagtgaacaggcccttaattgaccaataatctctgaaatagactgataaaatcgcatgatttttgttttagtattttttttactactactagaattcatggtggttgattttttttttctccggtgtgattgaatggtgcagcggcggaggaagtggaggaggaagagggcggagcaaggggtctacgtggagctgagcgaggcgatggactgcctgagccacatctgcacggaggggtgcacggaggtcgggccggtggggcgtgcgccggcggcggcgccgtgcccggcgtacgcgacggcgtgccggggactgcagctgctgatccgccacttctcccggtgccaccgcaccagctgcccgcgctgccagcgcatgtggcagctgctccgcctccacgccgccctctgcgacctccccgacggccactgcaacacccctctctgcatgtgagtgatcacaatgtcacactcacacacccataaatgcgtcttttaaacatacgcttgaaaagatgaaaactagcgatatcatctaatctcactaaaatatcgttgaaagtctaaatgcgtatgcgtaatattcaattagcttacattgatctggttttgacttgggaaaaaaaaatctttgcattgtgtattgcaggcagttcaggagaaaggaggaggagaaggcggcagcgaaggcgaaggcaaaggccggcgatgatgacgataagtgggggcttctggtgaagaaggtgagggttgccagggccatgtcttctctgggcaagaggaggcagatgagttgctcccagtgctgagcaagtgcaatcacagcatcagcctgagacagtcaagagaagagtattgtagtatagaagggtactactatgtagttaggcatagtgcatgagtgtattgaagtgagctattttttggtttgttcaagatggtttggtctctcaagtttgagttgctttactgtcttcaatttgtagagttcttttgtgaggtgagagaaatgggctgaaatctgttgtaaaattgcctatatcgtaaataaacttttaatagtgaggtatatgatcattacagtttcatacctggatgattacattgataga

SEQ ID NO: 12 - protein, 347 aa, Oryza sativa
MCEGVRAAGDAAAAADVDVITSSGRRRIPAHSTVLASASPVLESILQRRLKKERDAAAGGGKVRRAVVLIRGVTDDAAAAFVRLLYAGSSGDEEEIDEKSAAQMLVLAHAYRVPWLKRRCEGAIGSRLTAESVVDTMQLAALCDAPQLHLRCTRLLAKEFKAVEKTEAWRFLQENDPWLELDILQRLHDADLRRRKWRRKRAEQGVYVELSEAMDCLSHICTEGCTEVGPVGRAPAAAPCPAYATACRGLQLLIRHFSRCHRTSCPRCQRMWQLLRLHAALCDLPDGHCNTPLCMQFRRKEEEKAAAKAKAKAGDDDDKWGLLVKKVRVARAMSSLGKRRQMSCSQC

SEQ ID NO: 13 - DNA, 3981 bp, Zea mays
aattggcctggctacccagacaagagcacgcaacgcaagcaactgcgacgtctcccgcccccgccgcgccccggaccttttccgcagatattttttttatccccccacgcccccggctcgttcccttccattccaccacctctcgccagacaacgagatgtgcgaggcaccgcggctcggcaaccgcgccggcgccggcgacccggccgccgccgacgtcgacgtcgtcacgacgggcgggcgccggaggatccccgcgcattcctctgtcctggtaagcgtccgatccctttttccccccctccttggcgcattgctccgttcggccgtcccgtccgggtcgtctagcagtcgtctgacgggagctgtgtccgtctctgtttccgcgcgcgctaggcctcggcgtcgccggtgctcggcagcatcctggagcgtcgcctgcggaaggacagggagagcggcaagcccgggcggtccgtcgtccggatccgcggcgtcaccgacgacgccgccgcggccttcgtccgcctcctctacgccggcaggtaacgcccgtgggcaccgtgcgtgcctgtctgtctccctttcaattccccggtcgcgcgcgctcagccatggcggcctccttgttgctagtcttgttttcgcacatgttagttcctcgcgaggctgcggccgcctggtccccggcctggggctggacaagccagtgcggcacacgattattagcaaggactaatcctgcgttattctaaacgcacgaggaggggaaacatggcgcgcgtgtgtgctccacccacacgttaactgcccggaagatcaacctttattcttagacatcatccagaagcgtttttatattgaaaaaaagtattctagtacttttttattctttatttatctcgagtccatctgtacgtttccttctttaacagtatatacacacagtataaacctgaaattcgaggtgtccaacgttctaggaatggcaagaagcaagtccatgaccacgcgctagcttcaacaatcgattttaaaactttgagtaggtaccaaatccaaccaaaccgatcgagctcccccgtagtaggaaaaccttgcacccaaattaagcattccccggacactatatctactataaactacggatcgaaatgcatttgaacagcggttccggacactatctagcgcttgtcggctcaacgagccaatcccgtgatattttttttttgccgcaccatccaatcaccgagccgggtcaggtcgcaaatcaaatggggctctcgttggtttgggttggccaactggctgcctcaccatcatctgtcagactgtcacgtcccgggttggcgcggcggcaggtcgcagccacgtcgcccgccgaacccctcttacaaccagagccacacacacaccccgcgccggtctgccaagtgcccaaacccccgtagcggacgccaaaaaatctcgtggctttcccagcggcgaataggacggcgacggagggcaaaaaaaaaagataaagctaaagcgaggtaaaaaaagcgttacaacctttttaagttattattattattattatttccccgccgctaggctgcctggttggtgtgggtgtggcactgtgggttgtgtgggtggcacctgcccggttggacctgcacgaccccgcgctctcagccgacgtgcgcgtcatcgcgtagcgcacgactgggccgggccgagccgtcgctgtcgcggggacggccggcgccgggacgcaacgcaccggcggaccactctcgcccacggctgctatcattcattgtggtgttgcttaccgtggattggaaaattcttcggccgacgtggctacatgcggattccgagaaaacgaccgcccgagcgatcgcagcctcgcgccctgggcaaatgttctttcttgtggaacccgtcgaaggttcttctaccacactagtacactactacctgactaccaggtcgaagtgctatacaactagtacggcatacgattcgtccaccacactagcgcatgttcttgtggctcacaggaacttgtcaggttctaactaaacagtgtctgcaacgctgtccgtgcttaacaatttgttagattttggatgatgacgaacgatcatgtgcttaacacgacagcaaaaacccgtctgggcaggtgcggcgaggatgaggatgacgacatggaggagcacgcggtgcaggtgctggtgctggcgcacgcgtaccaggtgccgtggctgaagcgggcgtgcgagggcgccatcggcgcgcgcctcaccgcggactcggtggtggacgtgctgcagctggccggcctctgcgacgcgccgcgcctgcacctgcgctgcgccaggctgctggccaaggagttcgcggccgtggagcgcaccgaggcctggcgcttcctgcaggagaacgacccctggcaggagctccacgtcctgcagcgcctgcacgaggccgacatggtacgcacgcttttttttcttcttccatggattgctaccggaccggaccggaccggaccacggtcggtcgatgtaaatctgactgacacatgcttgcacgcaccgcaccgaatggaagtgcgagtgcgaagaaattatcatgcatgcatgcatgatggcgcacgccggcggggtggggtgggcactggggagcatgctttctttaccgtcaagcttttgttccaggaaacggtggacatggtcggacctccgtgtcgcgttcgtgcgcgtacggcgtacgaacgacatgacgccagtgttcttttctatgtggttggtgcgtctgtttgggccggcccgtccacgccgaatggtcgggcgaatgttcgcatctctccgacctcttacctttccgactctgttttgattttacacgttttcatctggctgggggtagacaaatcgtgagaccaagtaatttgtgccttggtttgaccaaagttagcagaaatcttggcggctactccatcagttccatctacaaacattcttcgtagggcttcaaaagtccagcggagaacgtttcaccaataataacgtttgttggaaacaacagttccacgatgcacgtttttttttgtgtaccgacgaccgacccagcctcttagctaaagcactgacccgatgcgcgctcggttccttcctggacatgcagcggcggcgcaagtggcggcggaagcgcgcggagcagcgcgtgtacatggagctgagcgaggccatggactgcctggaccacatctgcacggagggatgcacggaggtcggcccggcggggcgggcgccggcgccggcgccgtgcgcgcgctacgccacgtgccggggcctgcagctgctcatccgccacttctcccagtgccaccgcaagagctgcgcgcggtgccagcgcatgtggcagctgctccgcctccactccgcgctctgcgaccgtaccgaccgctgcaacaccccgctctgcatgtaagcgccgcgccgccgcttgcttcgttgcagccatccatggtccattgcgtgacctcgcgcctcacattgttgtgcttgtgccgtgcaggaggtttaagcagcaggagcaggataaggcggctgccaagggcggcgatgatggcgacaaatgggggcttctggtgaagaaggtgaagactgccagggtcttctcttctctggccaacaggaagcagatgagcaccaccaccacccagtgctgaggagcagcctgggatgcttaggggtcaaagttagattagtgagagagaatgaagcgagcagtttttttttgggtctgttttgagatgtcagatctcttttgagatatgtacatgtacatgcctactttgtcaccctacgttggtatagctcttttatcgaggtgggagttggtcagccggaagtctatgtaaaaagtgcgcatgtatcattatataataatagggaaatatgagaagtattttagtcttatgat

SEQ ID NO: 14 - protein, 344 aa, Zea mays
MCEAPRLGNRAGAGDPAAADVDVVTTGGRRRIPAHSSVLASASPVLGSILERRLRKDRESGKPGRSVVRIRGVTDDAAAAFVRLLYAGRCGEDEDDDMEEHAVQVLVLAHAYQVPWLKRACEGAIGARLTADSVVDVLQLAGLCDAPRLHLRCARLLAKEFAAVERTEAWRFLQENDPWQELHVLQRLHEADMRRRKWRRKRAEQRVYMELSEAMDCLDHICTEGCTEVGPAGRAPAPAPCARYATCRGLQLLIRHFSQCHRKSCARCQRMWQLLRLHSALCDRTDRCNTPLCMRFKQQEQDKAAAKGGDDGDKWGLLVKKVKTARVFSSLANRKQMSTTTTQC

SEQ ID NO: 15 - DNA, 1504 bp, Triticum aestivum
gagcctccctcccccgaaaccaacaccacacgcacgccgcgacatttcccagatatttcgcccctgcgcttttcttcctcctcccttccttccttctccgccgcaattgctcctagttatccttagcacagcatgccggaggcacctgcacgggcagccggcggcggcggcggcgcctccggccccgccgacgtcgacgtcgtcacctccagcggccgccgcaagatcgccgcccattcctccgttcttgcgtcggcgtcgccggtgctggagaccatcctggagcgccggctgcagagagtcagggagagcggcaagggcggcagggccgtcgtccggatccgcggcgtcaccgacgacgtcgcggcggcgttcgtccgcctcctctacgccggcagcaggcgtggcgatggtgaggtggaggaggaggtggagaggtacgcggagcagctgctggtgctggcgcacgcgtaccgggtgccgtggctgaagcggtggtgccaggaggccatcgggtcgcggctcaccccgggcaccgtggtggacgcgctgcagctggccgacctctgcgacgcgccgcagctgcacctccgctgcatgcgcctgctcgccaaggagttccgcgccgtcgagcgcaccgaggcatggcgcttcctccgcgacaacgacccctggcaggagctcgacgtcctccgccgcctccacgacgccgacatgcggcggcgaaagtggcgtcggaagcgcgcggagcagaaggtgtacgtggagctgagcgacgccatggacatcctgcggcacatctgcacggagggctgcacggaggtcggccctgtggggcaggcgccggccaagtcgccgtgcccggcgtacgcgacgtgccggggcctgcagctgctcatccgccacttctcccggtgcaagagccgcgccacctgcccccgctgccagcgcatgtggcagctgctccgcctccacgccgcgctctgccgcgtccccgacggccactgcaacactcctctctgcacgcagttcaagctcaaggagcagcagaaggaggcgatgtcggcttcggtggcggcgaaggccggcgacggcaggtgggggcttcttgtgaagaaggtgaaggctgtcagtgtcatgtcttccctcggcaagagaagctcgccctctcagtgctgctgagcctgagcgaatggaatgtgtgttactgaggatcccctctcagagctgatcgaatgttgtgcaatcacaacgagaatcagcctgggacaggtagctagatgtagtgtgcgagtgtagtgaaagcgggctattttttggcccgtctttccagtgtataatctctctattgagcaatgtacattagttatatcaatcatagtgttattcttttgtggggcgagagaggaaaagggtggagatttgttgtaaaatactgcctgcactgtaatactagtagcaaagtatgggatttgtccacatagcaaaaaaaaaaaaaaaacga

SEQ ID NO: 16 - protein, 319 aa, Triticum aestivum
DVDVVTSSGRRKIAAHSSVLASASPVLETILERRLQRVRESGKGGRAVVRIRGVTDDVAAAFVRLLYAGSRRGDGEVEEEVERYAEQLLVLAHAYRVPWLKRWCQEAIGSRLTPGTVVDALQLADLCDAPQLHLRCMRLLAKEFRAVERTEAWRFLRDNDPWQELDVLRRLHDADMRRRKWRRKRAEQKVYVELSDAMDILRHICTEGCTEVGPVGQAPAKSPCPAYATCRGLQLLIRHFSRCKSRATCPRCQRMWQLLRLHAALCRVPDGHCNTPLCTQFKLKEQQEAMSASVAAKAGDGRWGLLVKKVKAVSVMSSL